(19) United States
(12) Patent Application Publication
(10) Pub. No.: US 2005/0118606 A1
(43) Pub. Date: Jun. 2, 2005

(54) Methods for identifying risk of breast cancer and treatments thereof

(76) Inventors: Richard B. Roth, La Jolla, CA (US); Caridad Rosette, San Diego, CA (US); Mikhail F. Denissenko, Poway, CA (US); Matthew Roberts Nelson, San Marcos, CA (US); Rikard Reneland, San Diego, CA (US); Andreas Braun, San Diego, CA (US); Stefan M. Kammerer, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/723,670

(22) Filed: Nov. 25, 2003

Related U.S. Application Data
(60) Provisional application No. 60/429,136, filed on Nov. 25, 2002.
(60) Provisional application No. 60/490,234, filed on Jul. 24, 2003.
(60) Provisional application No. 60/467,823, filed on May 2, 2003.

(51) Int. Cl. C12Q 1/68; A61K 48/00

(52) U.S. Cl. 435/6; 514/44

(57) ABSTRACT

Provided herein are methods for identifying a risk of cancer in a subject and/or a subject at risk of cancer, reagents and kits for carrying out the methods, methods for identifying candidate therapeutics for treating cancer, and therapeutic methods for treating cancer in a subject. These embodiments are based upon an analysis of polymorphic variations in a KLF12 nucleic acid, which has a nucleotide sequence exemplified in SEQ ID NO: 1, 2 or 3.

### RELATED PATENT APPLICATIONS

This patent application claims the benefit of provisional patent application No. 60/429,136 filed Nov. 25, 2002 and provisional patent application No. 60/490,234 filed July 24, 2003, having attorney docket number 524593004100 and 524593004101, respectively. This patent application also claims the benefit of provisional patent application No. 60/467,823 filed May 2, 2003 and having attorney docket no.524593005200. Each of these provisional patent applications names Richard B. Roth et al as inventors and is hereby incorporated herein by reference in its entirety, including all drawings and cited publications and documents.

### FIELD OF THE INVENTION

The invention relates to genetic methods for identifying a risk of cancer and treatments specifically targeted to the disease.

### BACKGROUND

Breast cancer is the third most common cancer, and the most common cancer in women, as well as a cause of disability, psychological trauma, and economic loss. Breast cancer is the second most common cause of cancer death in women in the United States, in particular for women between the ages of 15 and 54, and the leading cause of cancer-related death (Forbes, Seminars in Oncology, vol.24(1), Suppl 1, 1997: pp.S1-20-S1-35). Indirect effects of the disease also contribute to the mortality from breast cancer including consequences of advanced disease, such as metastases to the bone or brain. Complications arising from bone marrow suppression, radiation fibrosis and neutropenic sepsis, collateral effects from therapeutic interventions, such as surgery, radiation, chemotherapy, or bone marrow transplantation-also contribute to the morbidity and mortality from this disease.

While the pathogenesis of breast cancer is unclear, transformation of normal breast epithelium to a malignant phenotype may be the result of genetic factors, especially in women under thirty (Miki, et al, Science, 266: 66-71 (1994)). However, it is likely that other, non-genetic factors also have a significant effect on the etiology of the disease. Regardless of its origin, breast cancer morbidity increases significantly if it is not detected early in its progression. Thus, considerable efforts have focused on the elucidation of early cellular events surrounding transformation in breast tissue. Such efforts have led to the identification of several potential breast cancer markers. For example, alleles of the BRCA1 and BRCA2 genes have been linked to hereditary and early-onset breast cancer (Wooster, et al.; Science, 265: 2088-2090 (1994)). However, BRCA1 is limited as a cancer marker because BRCA1 mutations fail to account for the majority of breast cancers (Ford et al., British J Cancer, 72: 805-812 (1995)). Similarly, the BRCA2 gene, which has been linked to forms of hereditary breast cancer, accounts for only a small portion of total breast cancer cases.

### SUMMARY

It has been discovered that multiple polymorphic variations in the KLF12 gene, which encodes a zinc finger repressor factor, and a region surrounding the gene, are associated with the occurrence of cancer, in particular breast cancer. Thus, featured herein are methods for identifying risk of cancer in a subject or a subject at risk of cancer, which comprises detecting the presence or absence of one or more polymorphic variations in a KLF12 nucleotide sequence in a nucleic acid sample from a subject, where the genomic KLF12 nucleotide sequence is set forth as SEQ ID NO: 1, and coding nucleotide sequences for two isoforms are set forth as SEQ ID NO: 2 or 3, or a substantially identical nucleotide sequence thereof. In certain embodiments, a polymorphic variation described hereafter is detected.

Also featured are methods for treating cancer in a subject by identifying subjects at risk of a cancer and treating the subject with a suitable treatment (e.g., administering a therapeutic molecule). In addition, featured are methods for preventing cancer in a subject by identifying subjects at risk of a cancer and implementing preventative measures before clinical signs of the disease first manifest.

Also featured herein are nucleic acids that encode a KLF12 polypeptide and include one or more polymorphic variants associated with cancer described hereafter, and oligonucleotides which hybridize to those nucleic acids. Also provided are polypeptides encoded by a KLF12 nucleic acid, which include a full-length isoform and a truncated isoform polypeptide, and fragments thereof. In addition, provided are methods for identifying candidate therapeutic molecules for treating cancer and related disorders, as well as methods of treating cancer in a subject by administering a therapeutic molecule described herein. In specific embodiments, the methods described herein are directed to specific types of cancer, which include but are not limited to breast cancer, prostate cancer and melanoma. Many embodiments described herein are directed to breast cancer.

Also provided are compositions comprising a breast cancer cell and/or a KLF12 nucleic acid, or a fragment or substantially identical nucleic acid thereof, with a RNAi, siRNA, antisense DNA or RNA, or ribozyme nucleic acid designed from a KLF12 nucleotide sequence. In an embodiment, the nucleic acid is designed from a KLF12 nucleotide sequence that includes one or more breast cancer associated polymorphic variations, and in some instances, specifically interacts with such a nucleotide sequence. Further, provided are arrays of nucleic acids bound to a solid surface, in which one or more nucleic acid molecules of the array have a KLF12 nucleotide sequence, or a fragment or substantially identical nucleic acid thereof, or a complementary nucleic acid of the foregoing. Featured also are compositions comprising a breast cancer cell and/or a KLF12 polypeptide, with an antibody that specifically binds to the polypeptide. In an embodiment, the antibody specifically binds to an epitope in the polypeptide that includes a non-synonymous amino acid modification associated with breast cancer (e.g., results in an amino acid substitution in the encoded polypeptide associated with breast cancer).

### BRIEF DESCRIPTION OF THE DRAWINGS

FIGS. 1A-1YYYY depict a nucleotide sequence of a KLF12 gene and surrounding region.

Two transcript variants encoding different isoforms have been identified for KLF12: a full-length (FL) form, or isoform a, and a truncated (TR) form, or isoform b. The full-length form comprises a molecule approximately 402 amino acids in length, and the truncated form comprises a molecule approximately 269 amino acids in length. Polynucleotide sequences encoding both the FL and TR forms of KLF12 are provided in FIGS. 2A-2B, respectively. FIG. 2A depicts the FL form of KLF12 cDNA reported as SEQ ID NO: 2. FIG. 2B depicts the TR form of KLF12 cDNA reported as SEQ ID NO: 3. Polypeptide sequences of both the FL and TR forms of KLF12 are provided in FIGS. 3A-3B, respectively. FIG. 3A depicts the FL form of KLF12 polypeptide reported as SEQ ID NO: 4. FIG. 3B depicts the TR form of KLF12 polypeptide reported as SEQ ID NO: 5. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

FIG. 4 shows expression of KLF12 FL, KLF12 TR, and AP2 is cells.

FIG. 5 depicts siRNA inhibition of KLF gene expression.

FIG. 6 illustrates effects of siRNA on T-47D cell proliferation.

FIG. 7 shows effects of siRNA on MCF-7 cell proliferation.

FIG. 8 depicts effects of siKLF on T-47D cell proliferation according to a Wst-1 assay.

FIG. 9 illustrates effects of siKLF on T-47D cell proliferation according to a CyQuant assay.

FIG. 10 shows morphologies of siRNA-transfected MCF-7 cells.

FIG. 11 shows effects of siRNA on proliferation of melanoma and prostate cancer cells.

FIG. 12 illustrates apoptosis of cells by cell staining.

FIG. 13 shows show proximal SNPs in and around the KLF12 gene for females. The position of each SNP on the chromosome is shown on the x-axis and the y-axis provides the negative logarithm of the p-value comparing the estimated allele to that of the control group. Also shown are the exons and introns of the genes in the approximate chromosomal positions. The graph indicates polymorphic variants associated with breast cancer in regions spanning positions 78265 to 123880 in SEQ ID NO: 1, spanning positions 78265 to 96535 in SEQ ID NO: 1, or spanning positions 117297 to 123880 in SEQ ID NO: 1 are in linkage disequilibrium.

### DETAILED DESCRIPTION

It has been discovered that a number of polymorphic variants in and around a nucleotide sequence encoding a zinc finger transcriptional repressor, known as Kruppel-like factor 12 (KLF12), is associated with the occurrence of cancer. KLF12 is a developmentally-regulated transcription factor and important regulator of gene expression during embryonic and adult tissue differentiation (Roth et al. Genomics 63:384-390 (2000)). The protein encoded by this gene is a member of the Kruppel-like zinc finger protein family that represses expression of the AP-2 alpha (AP-2a) gene by binding to a specific site in the AP-2a gene promoter. AP-2a is a developmentally regulated transcription factor that is involved in epidermal cell proliferation, and accordingly plays an important role in human cancer (Bosher et al PNAS 92:744-747 (1995)). It has been reported that KLF12 binds to regulatory element A32 in the AP-2alpha gene promoter and represses its expression by excluding a positive factor, KLF9 (Imhof, A. et al. Molec. Cell. Biol. 19: 194-204, 1999). A variant of KLF12, which was isolated from hematopoetic stem cell progenitor cells, encodes a truncated (TR) isoform, which lacks the zinc finger DNA binding domain. Its function is unknown.

The transcription factor, AP-2, is an inducer of c-erbB2 oncogene expression in a nontumorigenic mammary epithelial cell line (ZR-75-1). A complex of AP-2a also stimulates the expression of IGF-1R and ER in mammary carcinoma cells. AP-2a is also known to induce bcl-2, an apoptosis suppressor. All these roles of AP-2a are relevant to the hyperproliferation associated with cancer. On the other hand, AP-2a is demonstrated to induce the cyclin dependent kinase inhibitor, p21WAF1/CIP1, resulting in cell cycle arrest (Zeng Y X et al. Nat Genet 1997 Jan.;15(1):78-82). This function of AP-2a may account of its tumor suppressor activity in cancer, including, but not limited to, primary breast cancer, malignant melanoma, and prostate cancer.

Featured herein is a polymorphic variant of KLF12 associated with an increased risk of cancer occurrence, which is useful for the early identification of cancer or a predisposition to cancer. A predisposition to cancer sometimes is indicative of a subject's increased susceptibility to developing cancer despite common environmental risk factors, or it sometimes is indicative of a subject's increased risk of developing aggressive forms of cancer more likely to metastasize or invade surrounding tissues, thus making the disease more difficult to treat. Accordingly, methods for treating or preventing cancer in a subject with a predisposition to cancer are described herein. Also featured herein are nucleic acids and fragments thereof that include one or more polymorphic variations associated with the occurrence of cancer, as well as polypeptides encoded by these nucleic acids. Also, associating KLF12 polymorphic variants with cancer has provided new, novel targets for design or discovery of therapeutic agents for cancer.

Cancers and Sample Selection

The term “cancer” as used herein refers to a condition characterized by uncontrolled, abnormal growth of cells. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. In specific embodiments, the cancer is breast cancer, prostate cancer or melanoma. All of the possible cancers listed herein are included in, or may be excluded from, the present invention as individual species.

Abnormal cells resulting from cancer are referred to as “neoplastic cells,” which are transformed cells that can form a solid tumor. The term “tumor” refers to an abnormal mass or population of cells (i.e., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize.

The term “invasion” as used herein refers to the spread of cancerous cells to adjacent surrounding tissues. The term “metastasis” as used herein refers to a process in which cancer cells travel from one organ or tissue to another non-adjacent organ or tissue. Cancer cells in the breast can spread to tissues and organs of a subject, and conversely, cancer cells from other organs or tissue can invade or metastasize to a breast. Cancerous cells from the breast may invade or metastasize to any other organ or tissue of the body. Breast cancer cells often invade lymph node cells and/or metastasize to the lung, liver, brain and/or bone and spread cancer in these tissues and organs. Breast cancers can spread to other organs and tissues and cause breast cancer, prostate cancer, colon cancer, ovarian cancer, cervical cancer, gastrointestinal cancer, pancreatic cancer, glioblastoma, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, ovarian cancer, neuroblastoma, myeloma, various types of head and neck cancer, acute lymphoblastic leukemia, acute myeloid leukemia, Ewing sarcoma and peripheral neuroepithelioma, and other carcinomas, lymphomas, blastomas, sarcomas, and leukemias.

Breast cancer is the rapid proliferation of abnormal cells in one or both of the breasts. While normal breast tissue cells reproduce and develop into healthy breast tissue, these abnormal cells proliferate rapidly and rarely form normal breast tissue. Instead, the abnormal cells proliferate, form tumors, and disrupt the breast, thereby decreasing breast function and eventually leading to death. There is a dire need for improved diagnostics and treatments directed to breast cancer. Currently there are 180,000 new cases of breast cancer diagnosed per year in the United States.

As used herein, the term “breast cancer” refers to a condition characterized by anomalous rapid proliferation of abnormal cells in one or both breasts of a subject. In breast cancer, neoplastic cells often are identified in one or both breasts only and not in another tissue or organ, in one or both breasts and one or more adjacent tissues or organs (e.g. lymph node), or in a breast and one or more non-adjacent tissues or organs to which the breast cancer cells have metastasized.

Breast cancers arise most commonly in the lining of the milk ducts of the breast (ductal carcinoma), or in the lobules where breast milk is produced (lobular carcinoma). Other forms of breast cancer include Inflammatory Breast Cancer and Recurrent Breast Cancer. Inflammatory Breast Cancer is a rare, but very serious, aggressive type of breast cancer. The breast may look red and feel warm with ridges, welts, or hives on the breast; or the skin may look wrinkled. It is sometimes misdiagnosed as a simple infection. Recurrent disease means that the cancer has come back after it has been treated. It may come back in the breast, in the soft tissues of the chest (the chest wall), or in another part of the body. As used herein, the term “breast cancer” may include both Inflammatory Breast Cancer and Recurrent Breast Cancer.

In an effort to detect breast cancer as early as possible, women regularly undergo physical exams and screening mammograms. A diagnostic mammogram is performed to evaluate a breast complaint or abnormality detected by physical exam or routine screening mammography. If an abnormality seen with diagnostic mammography is suspicious, additional breast imaging (with exams such as ultrasound) or a biopsy may be ordered. A biopsy followed by pathological (microscopic) analysis is the only definitive way to determine whether a woman has breast cancer. Excised breast cancer samples are often subjected to the following analyses: diagnosis of the breast tumor and confirmation of its malignancy; maximum tumor thickness; assessment of completeness of excision of invasive and in situ components and microscopic measurements of the shortest extent of clearance; level of invasion; presence and extent of regression; presence and extent of ulceration; histological type and special variants; pre-existing lesion; mitotic rate; vascular invasion; neurotropism; cell type; tumor lymphocyte infiltration; and growth phase.

The stage of a breast cancer can be classified as a range of stages from Stage 0 to Stage IV based on its size and the extent to which it has spread. The following table summarizes the stages:

TABLE 1

| | | Lymph Node | |
|---|---|---|---|
| Stage | Tumor Size | Involvement | Metastasis (Spread) |
| I | Less than 2 cm | No | No |
| II | Between 2-5 cm | No, or in same side of | No |
| | | breast | |
| III | More than 5 cm | Yes, on same side of | No |
| | | breast | |
| IV | Not applicable | Not applicable | Yes |

Stage 0 cancer is a contained cancer that has not spread beyond the breast ductal system. Fifteen to twenty percent of breast cancers detected by clinical examinations or testing are in Stage 0 (the earliest form of breast cancer). Two types of Stage 0 cancer are lobular carcinoma in situ (LCIS) and ductal carcinoma in situ (DCIS). LCIS indicates high risk for breast cancer. Many physicians do not classify LCIS as a malignancy and often encounter LCIS by chance on breast biopsy while investigating another area of concern. While the microscopic features of LCIS are abnormal and are similar to malignancy, LCIS does not behave as a cancer (and therefore is not treated as a cancer). LCIS is merely a marker for a significantly increased risk of cancer anywhere in the breast. However, bilateral simple mastectomy may be occasionally performed if LCIS patients have a strong family history of breast cancer. In DCIS the cancer cells are confined to milk ducts in the breast and have not spread into the fatty breast tissue or to any other part of the body (such as the lymph nodes). DCIS often are detected on mammograms as tiny specks of calcium (known as microcalcifications) 80% of the time. Less commonly DCIS can present itself as a mass with calcifications (15% of the time); and even less likely as a mass without calcifications (<5% of the time). Breast biopsy is used to confirm DCIS. Standard DCIS treatment is breast-conserving therapy (BCT): lumpectomy followed by radiation treatment or mastectomy.

In Stage I, the primary (original) cancer is 2 cm or less in diameter and has not spread to the lymph nodes.

In Stage IIA, the primary tumor is between 2 and 5 cm in diameter and has not spread to the lymph nodes. In Stage IIB, the primary tumor is between 2 and 5 cm in diameter and has spread to the axillary (underarm) lymph nodes; or the primary tumor is over 5 cm and has not spread to the lymph nodes.

In Stage IIIA, the primary breast cancer of any kind that has spread to the axillary (underarm) lymph nodes and to axillary tissues. In Stage IIIB, the primary breast cancer is any size, has attached itself to the chest wall, and has spread to the pectoral (chest) lymph nodes.

In Stage IV, the primary cancer has spread out of the breast to other parts of the body (such as bone, lung, liver, brain). The treatment of Stage IV breast cancer focuses on extending survival time and relieving symptoms.

Based in part upon selection criteria set forth above, individuals having breast cancer can be selected for genetic studies. Also, individuals having no history of cancer or breast cancer can be selected as controls for genetic studies. Other selection criteria may include: the sample was derived from a Caucasian; the sample was derived from an individual of German paternal and maternal descent; the case samples were derived from individuals diagnosed with breast cancer; the control samples were derived from individuals free of cancer and no family history of breast cancer; and sufficient genomic DNA was extracted from each blood sample for all allelotyping and genotyping reactions performed during the study. Phenotype information includes pre- or post-menopausal onset, familial predisposition to cancer or breast cancer, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), or method of treatment (e.g., surgery, chemotherapy, hormone therapy, radiation therapy).

Polymorphic Variants Associated with Breast Cancer

Genetic analysis provided herein linked breast cancer with a polymorphic variant of a nucleotide sequence located on chromosome thirteen that encodes a zinc finger repressor factor polypeptide designated KLF12. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length sometimes is 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

Furthermore, a genotype or polymorphic variant sometimes is expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of cancer, or a specific type of cancer such as breast cancer.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant sometimes is detected on either or both strands of a double-stranded nucleic acid. Also, a polymorphic variant sometimes is located within an intron or exon of a gene or within a portion of a regulatory region such as a promoter, a 5′ untranslated region (UTR), a 3′ UTR, and in DNA (e.g., genomic DNA (GDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

For duplex DNA, a polymorphic variation may be reported from one strand or its complementary strand. For example, a cytosine at position 96535 in SEQ ID NO: 1 can be reported as a guanine from the complementary strand. Also, while polymorphic variations at all positions within a haplotype often are reported from the same strand orientation, polymorphic variations at certain positions within a haplotype sometimes are reported from one strand orientation while others are reported from the other. The latter sometimes occurs even though it is understood by the person of ordinary skill in the art that polymorphic variants in a haplotype occur within one strand in a nucleic acid. Where a haplotype is reported from mixed strand orientations, a person of ordinary skill in the art can determine the orientation of each polymorphic variation in the haplotype by analyzing the orientation of each extension oligonucleotide utilized to identify each polymorphic variation.

In the genetic analysis that associated a polymorphic variation in KLF12 with breast cancer, samples from individuals having breast cancer and individuals not having breast cancer were allelotyped and genotyped. The term “genotyped” as used herein refers to a process for determining a genotype of one or more individuals, where a “genotype” is a representation of one or more polymorphic variants in a population. It was determined that both alleleic variants of the single nucleotide polymorphism (SNP) located in KLF12 nucleic acid at position 96535 in SEQ ID: 1 were present in the set of individuals tested in the genetic analysis described in Examples hereafter.

Additional Polymorphic Variants Associated with Breast Cancer

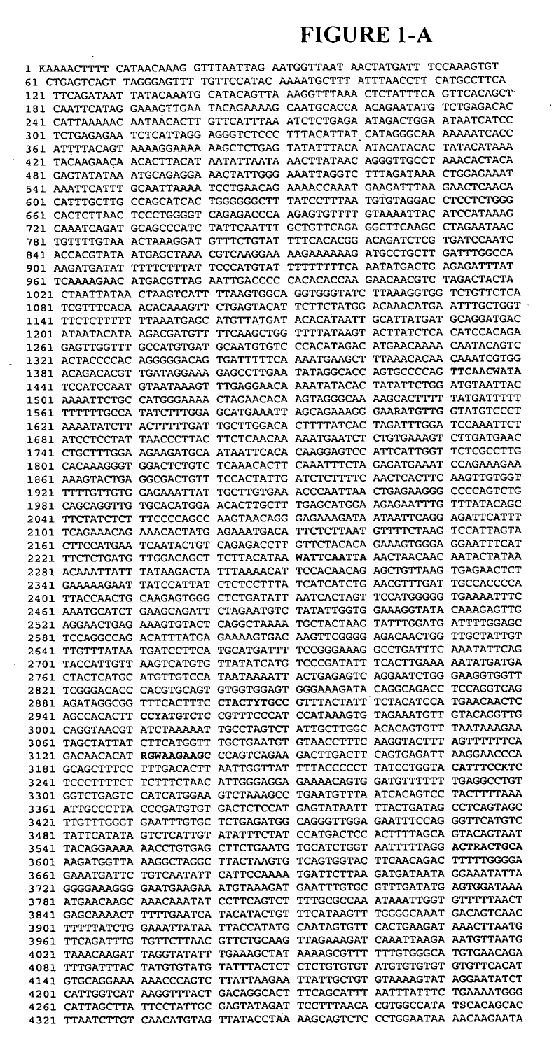

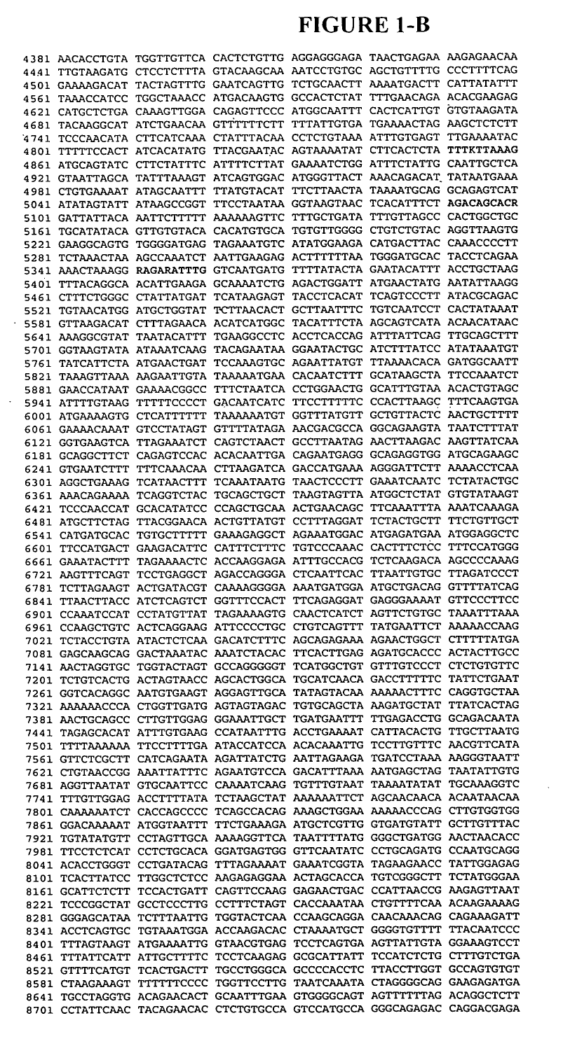

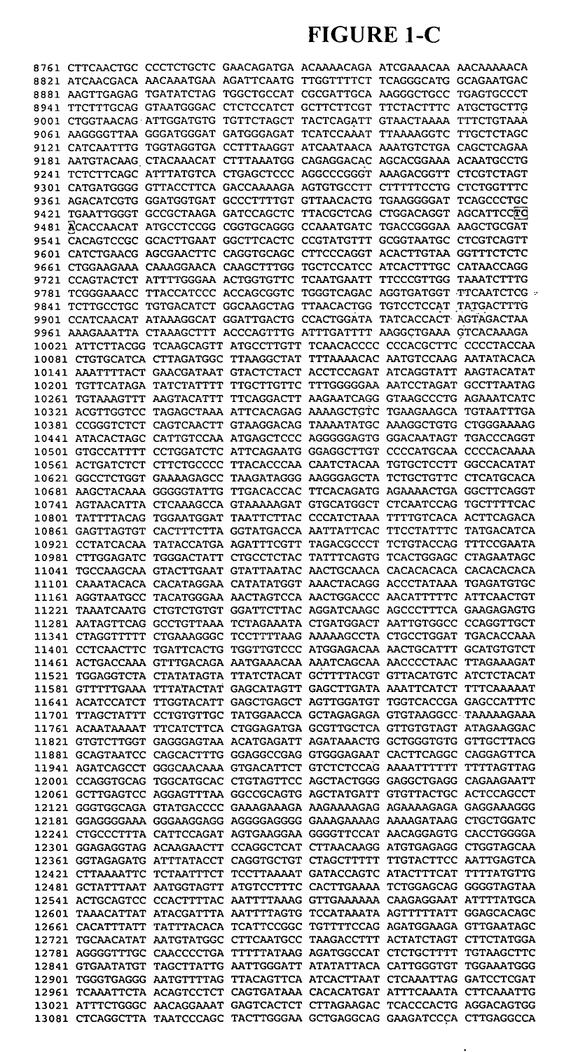

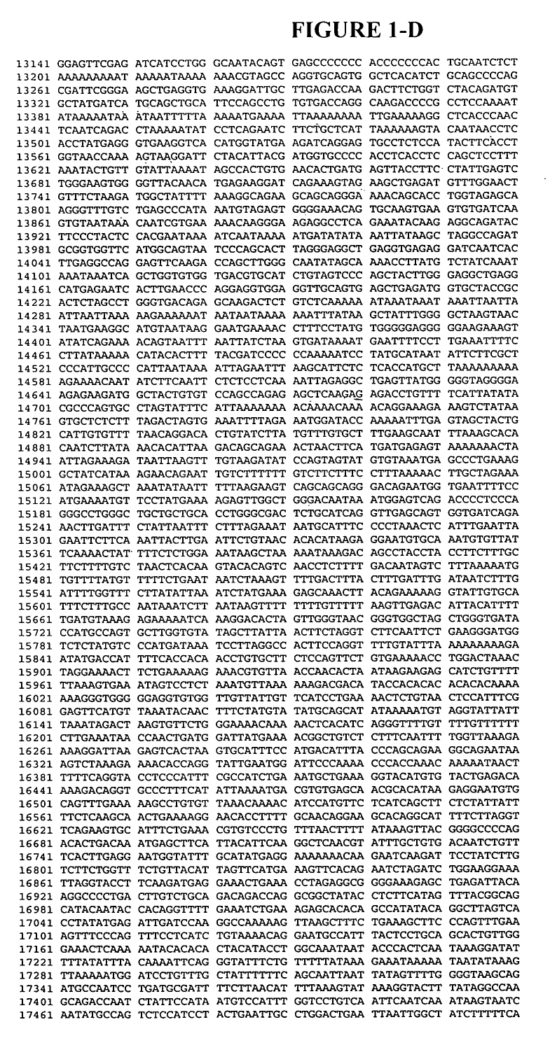

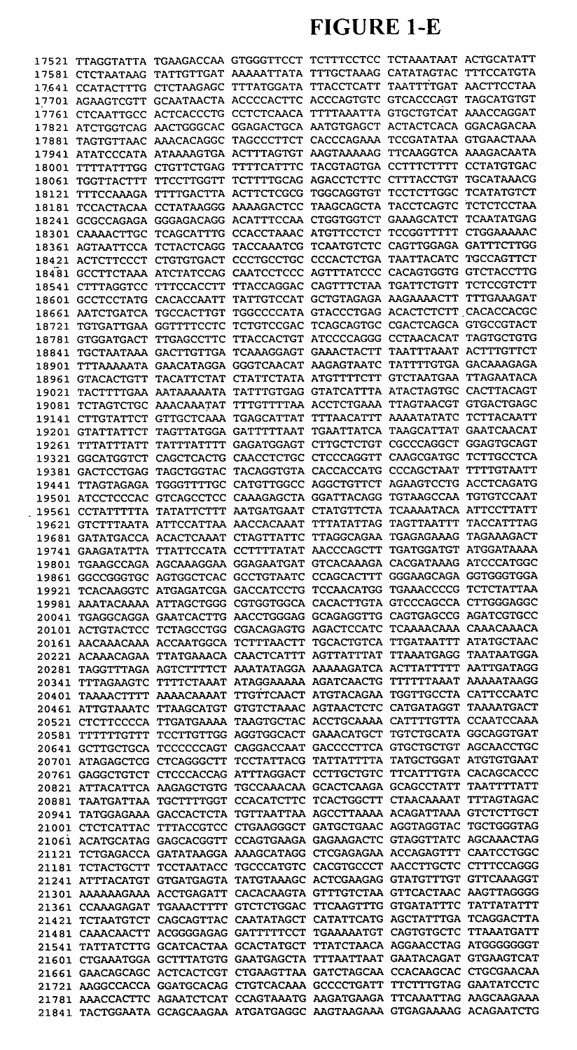

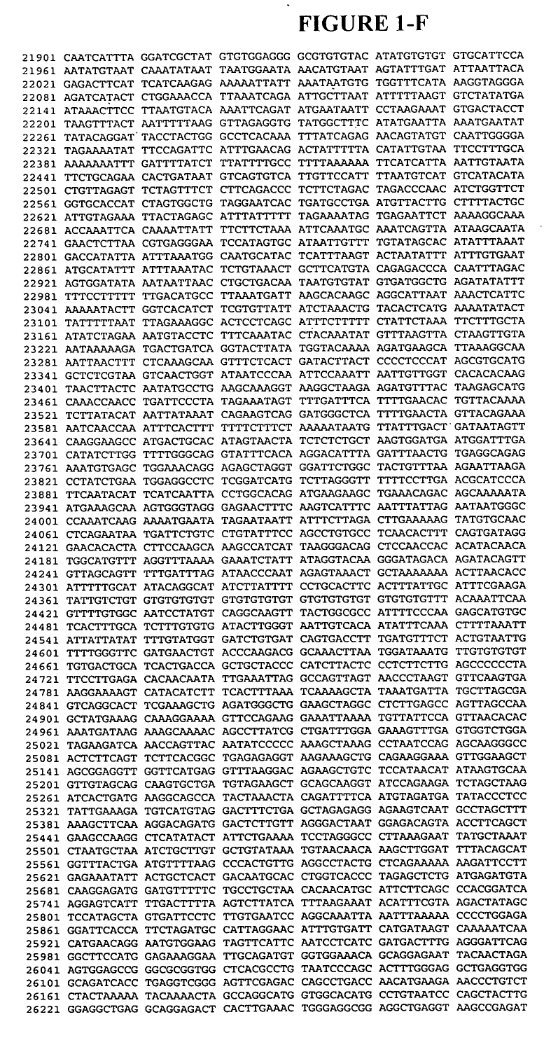

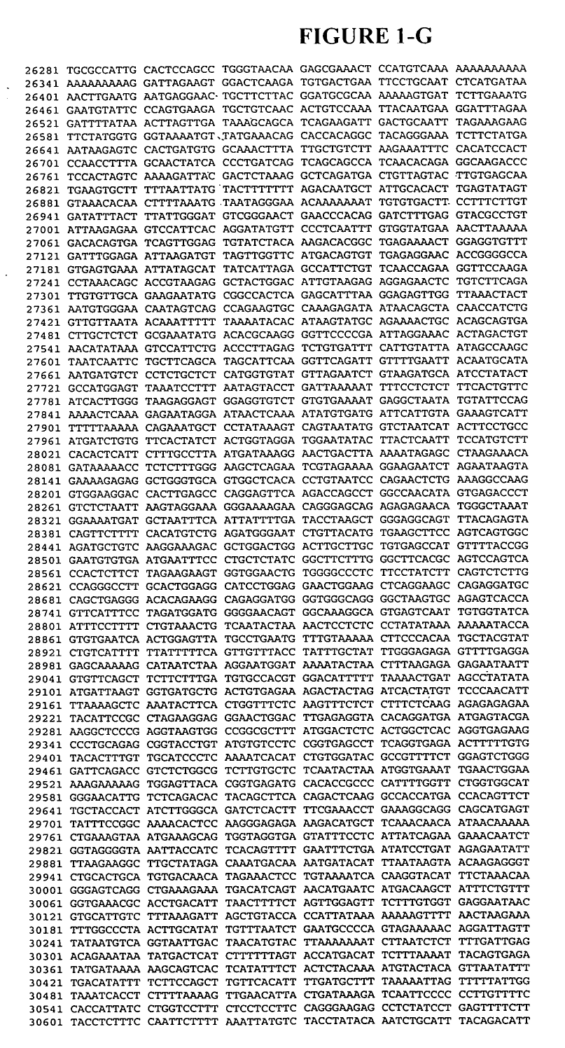

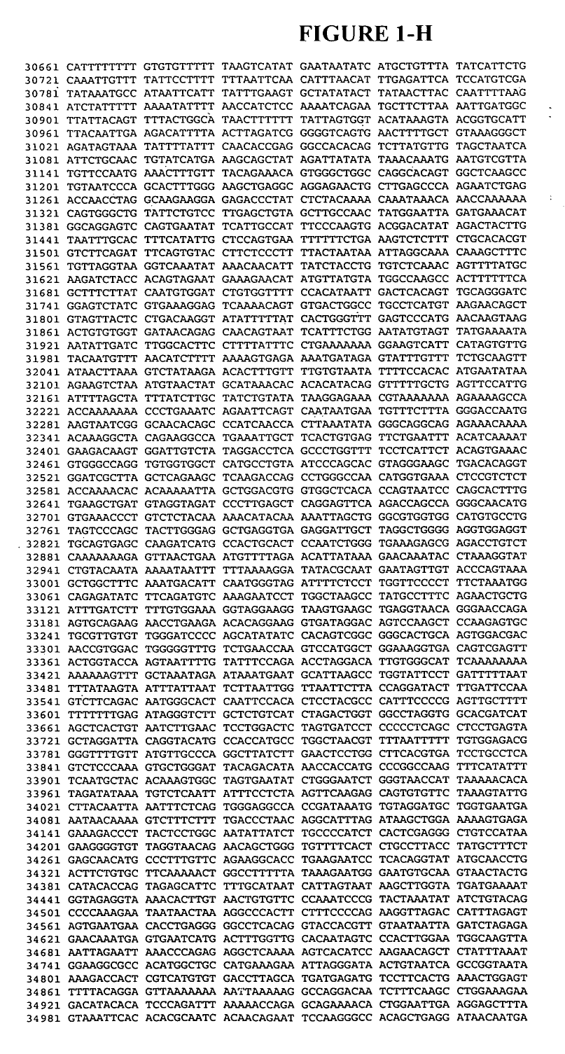

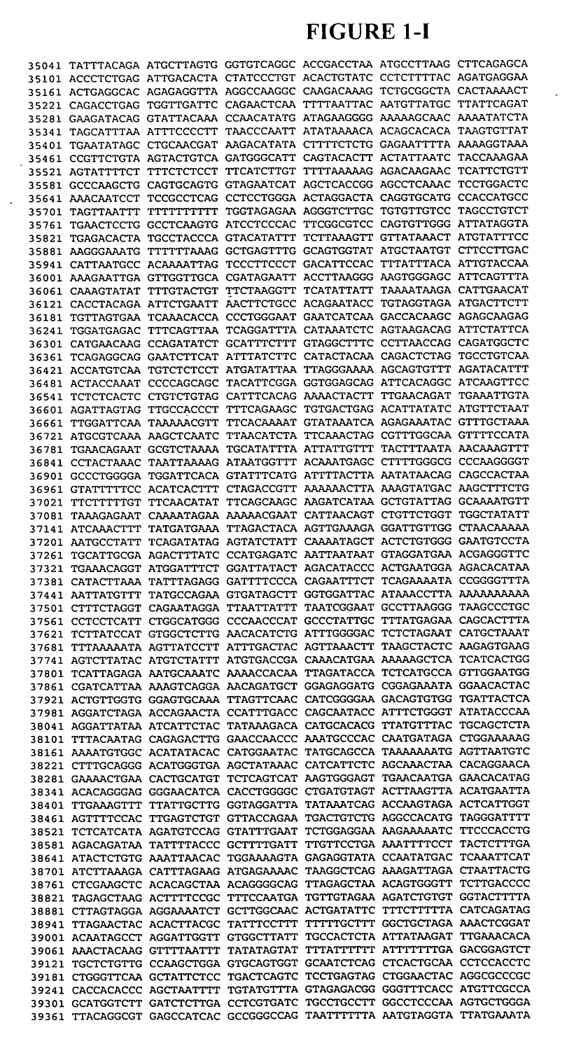

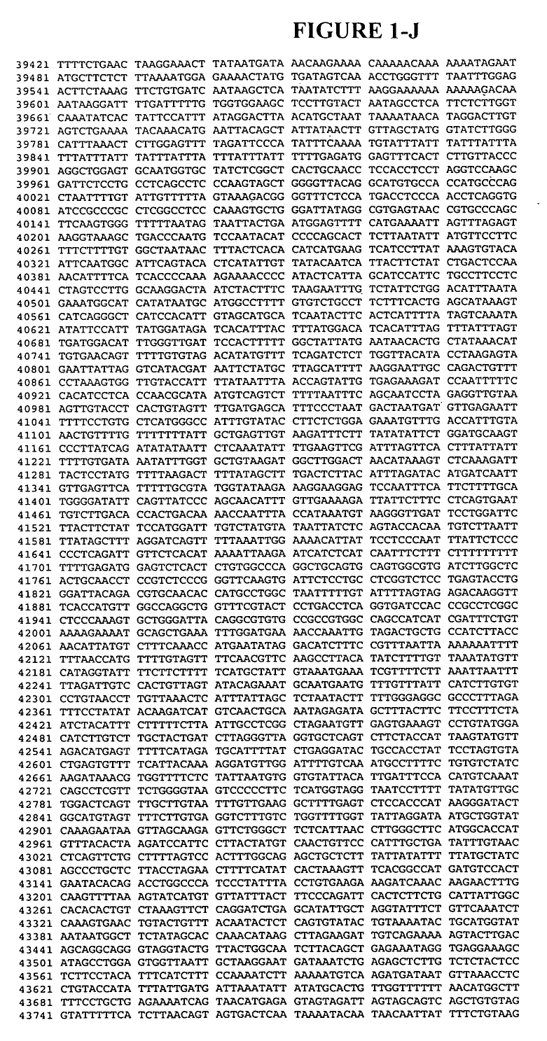

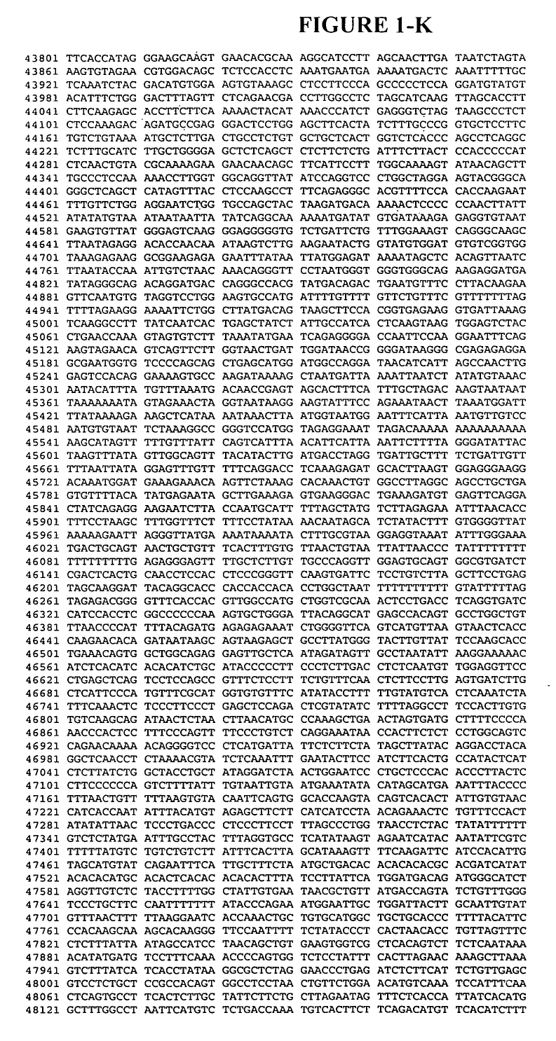

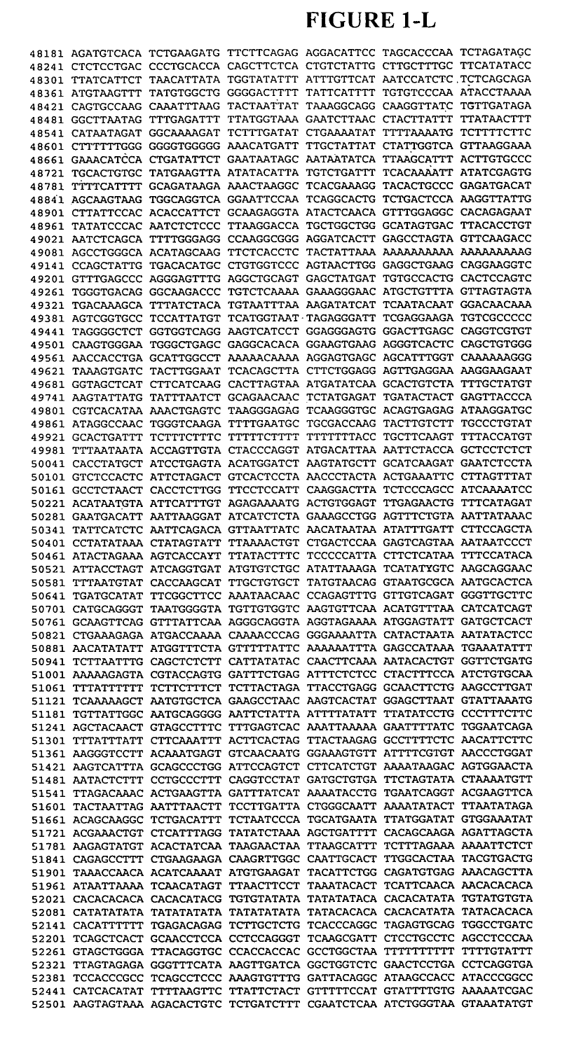

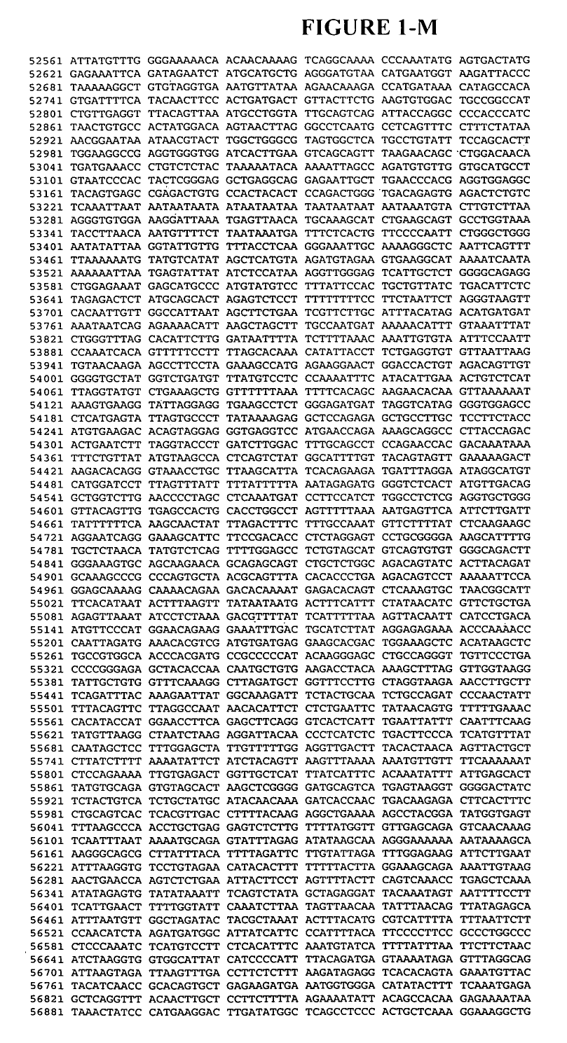

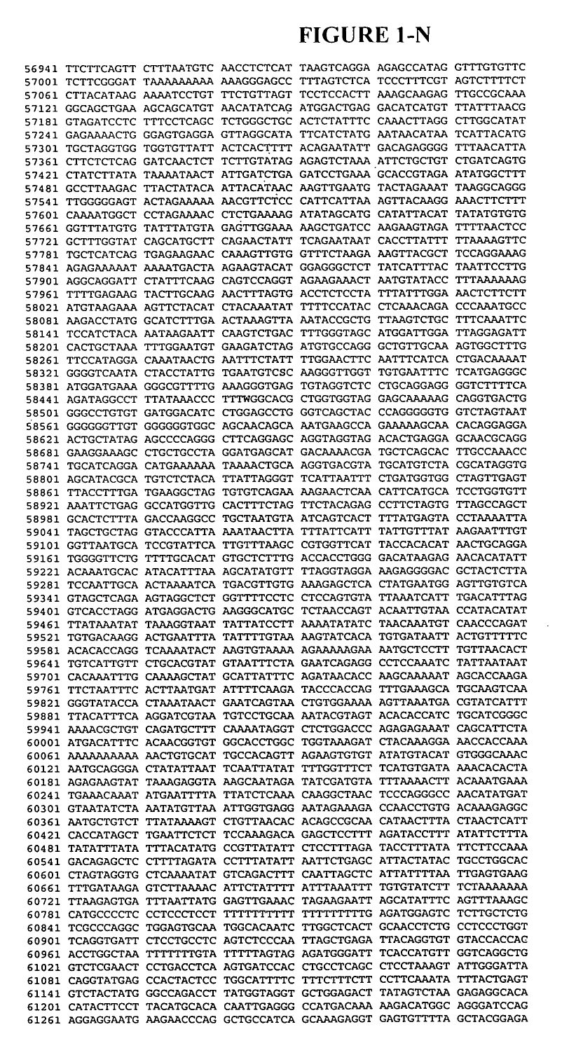

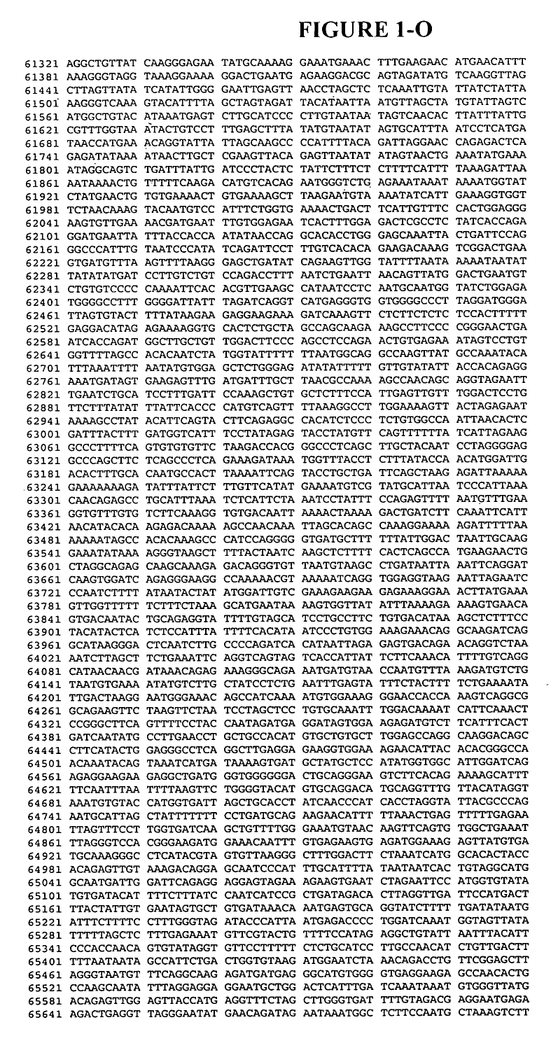

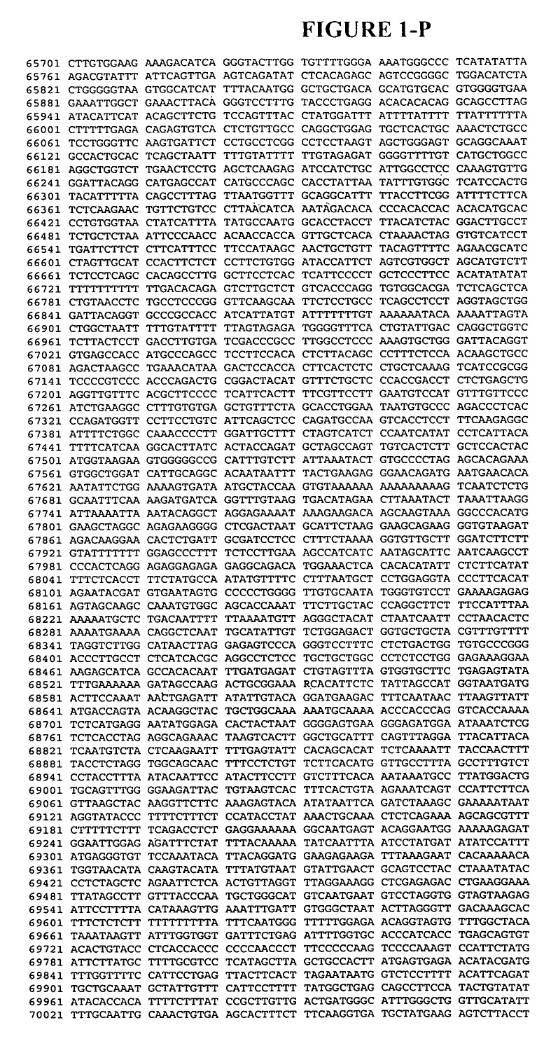

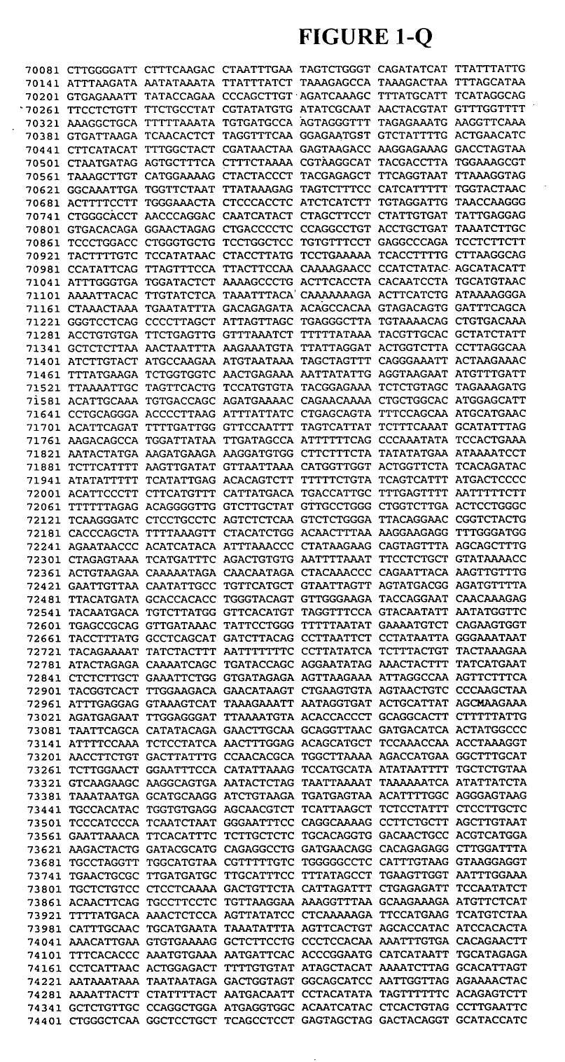

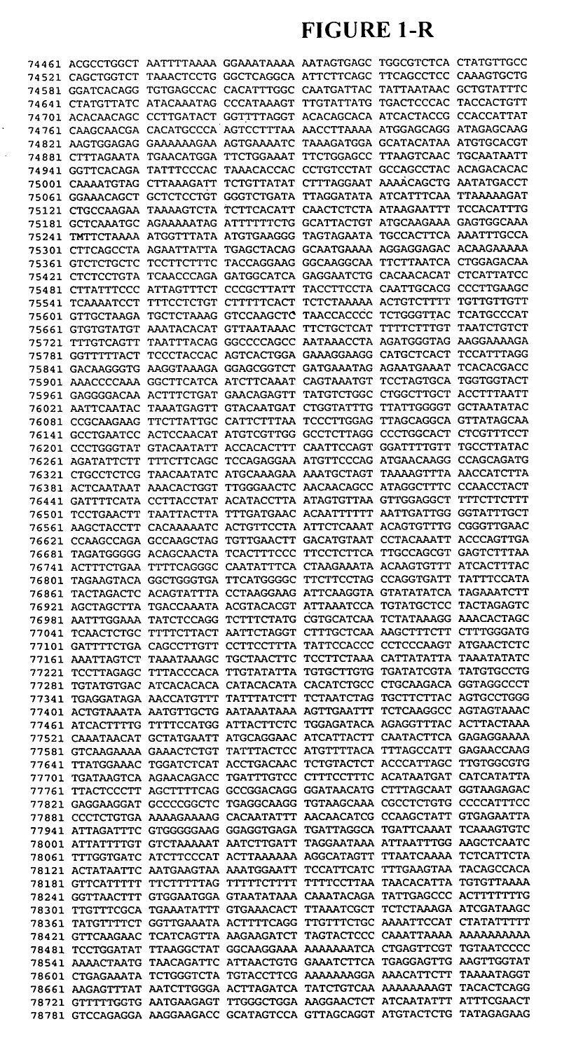

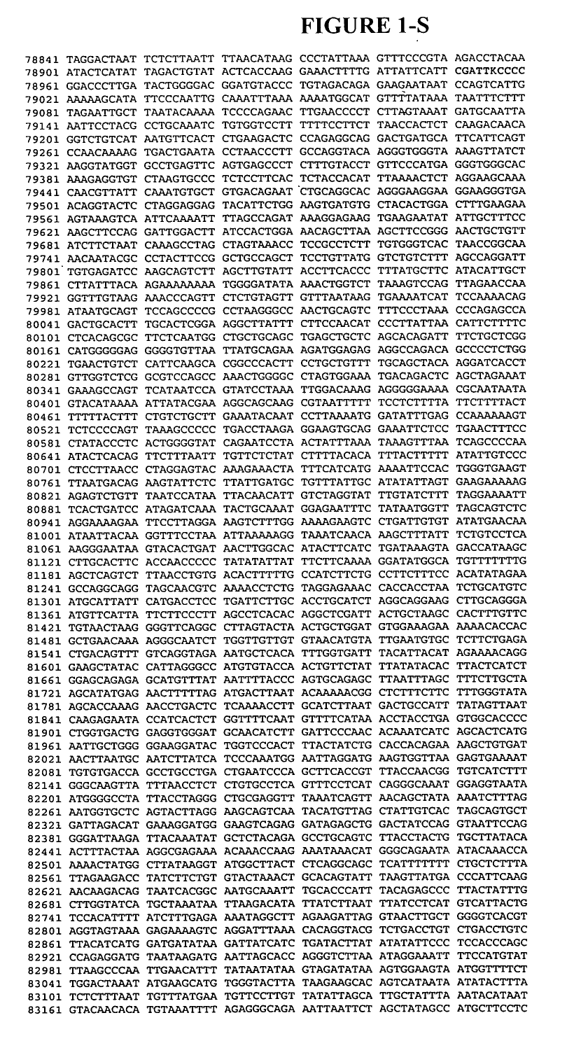

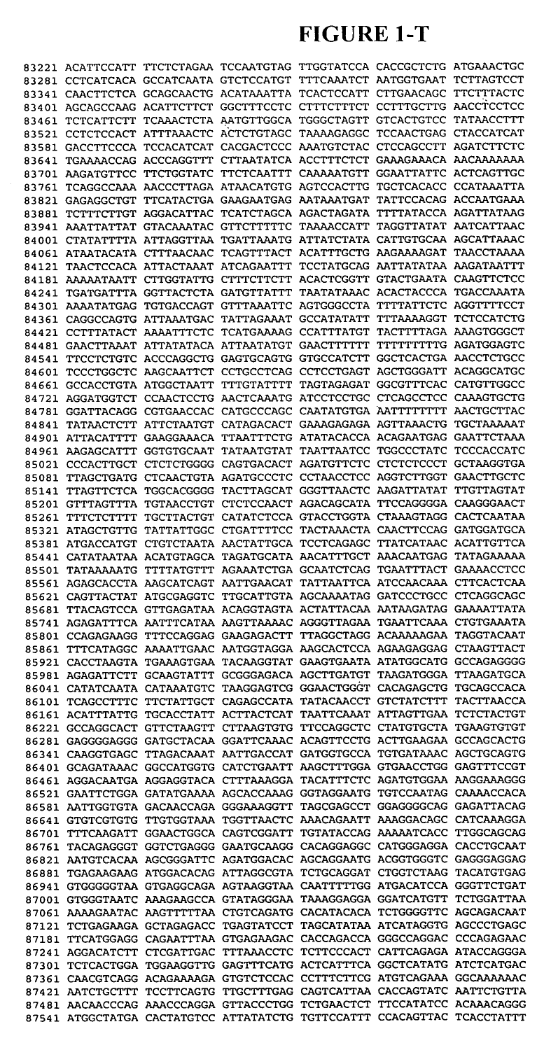

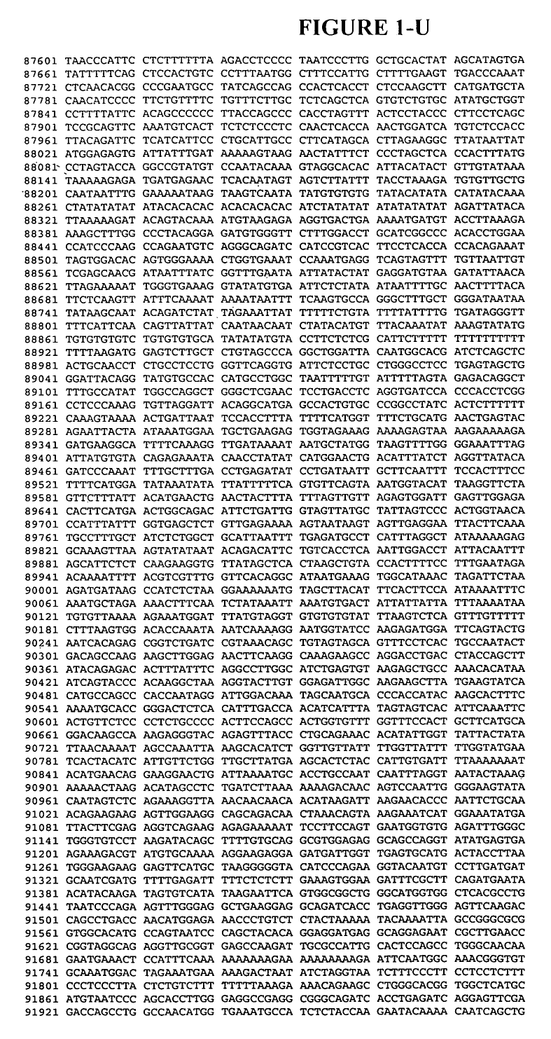

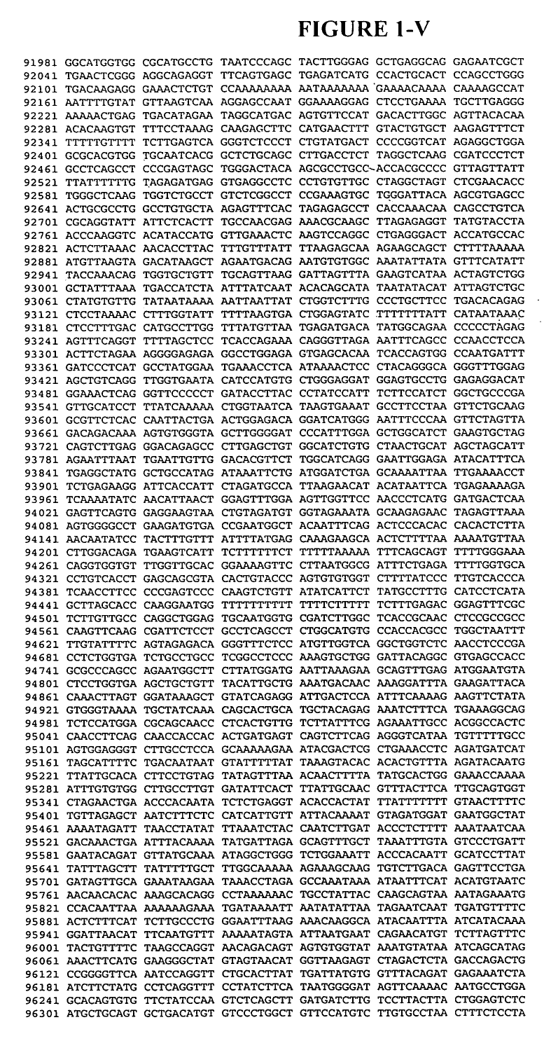

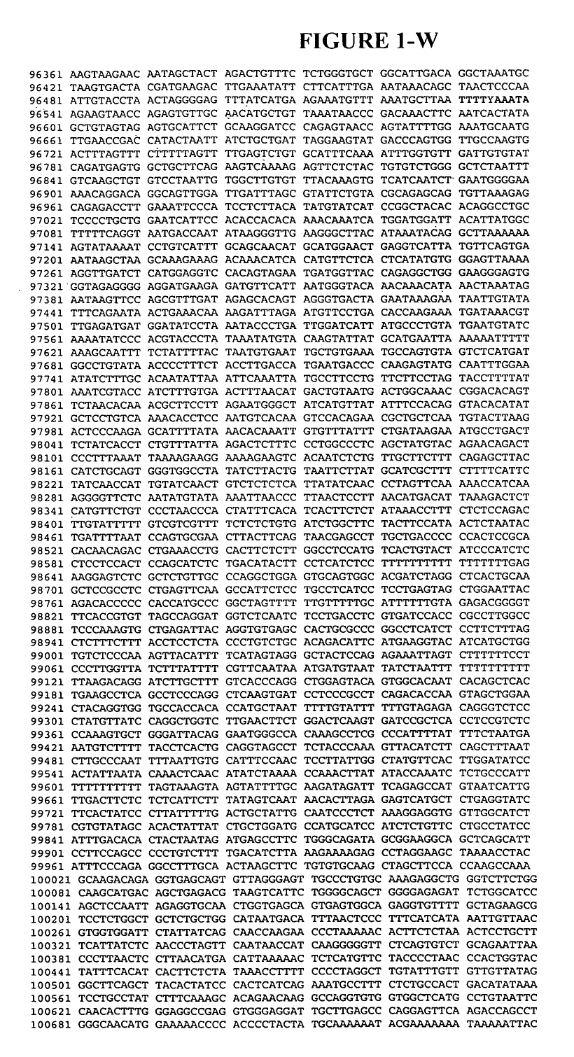

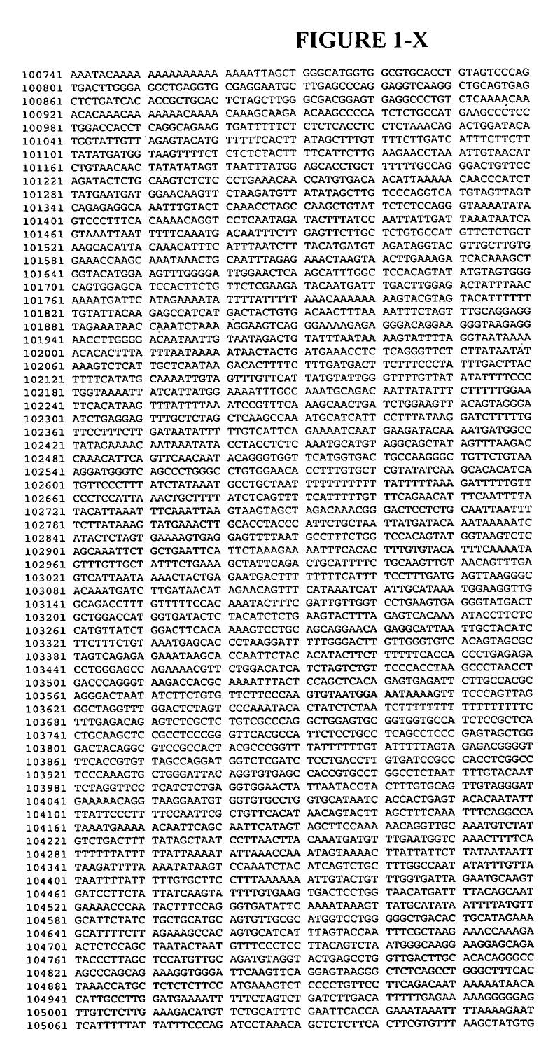

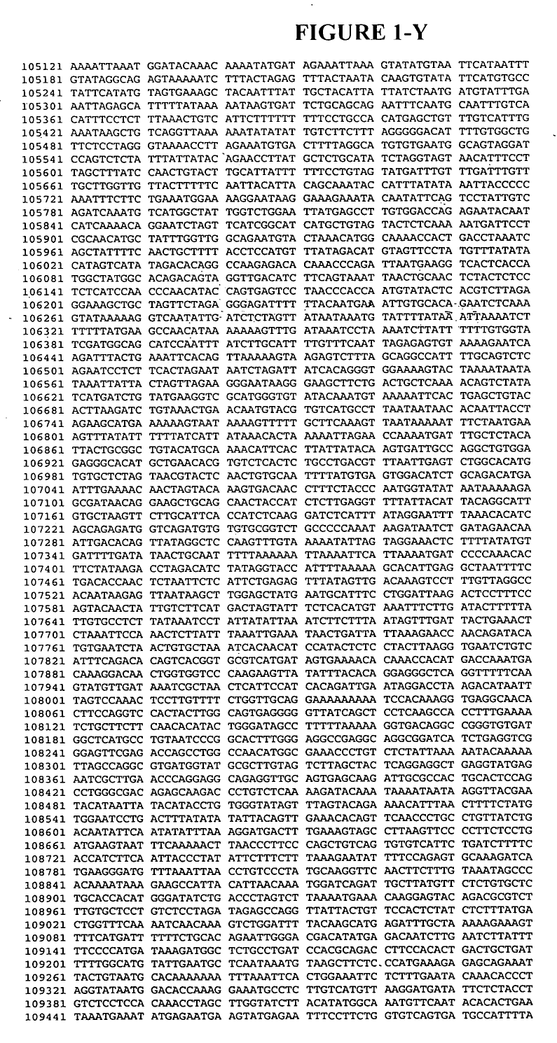

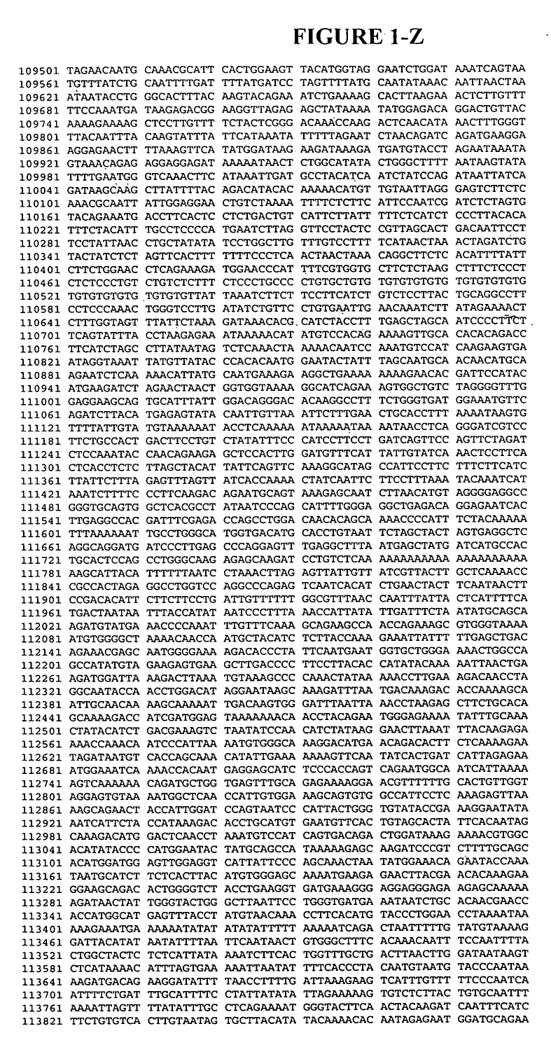

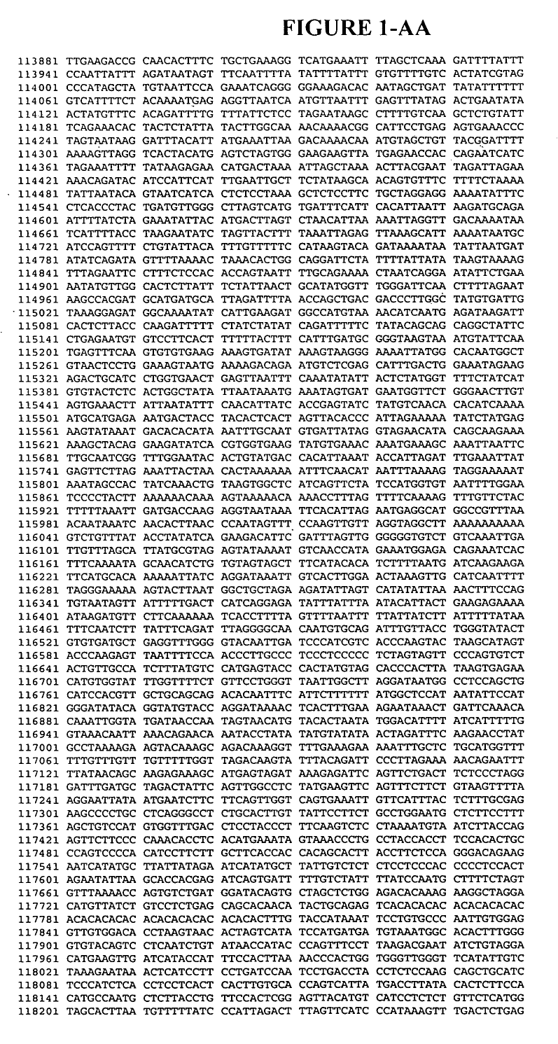

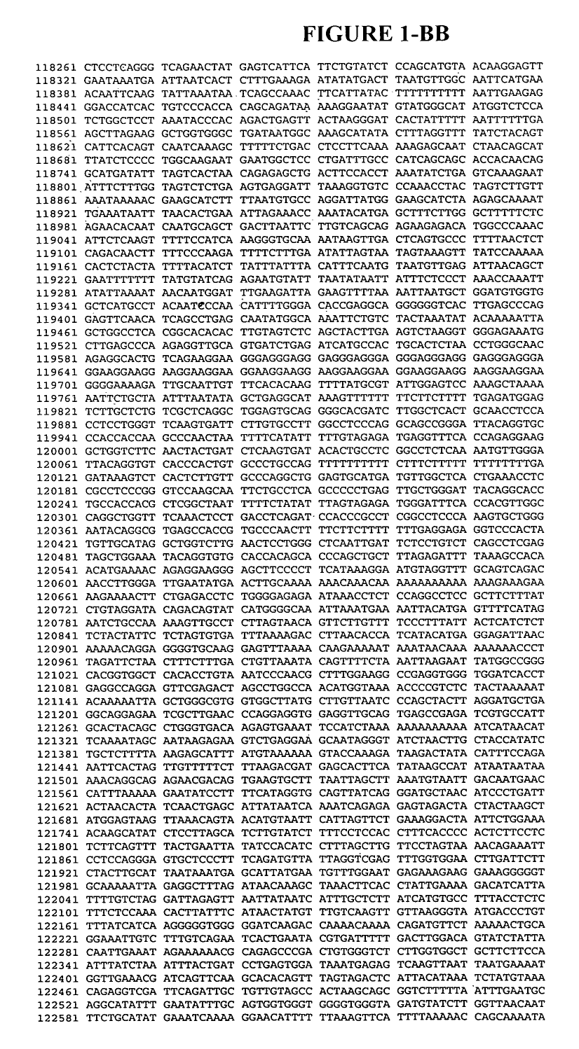

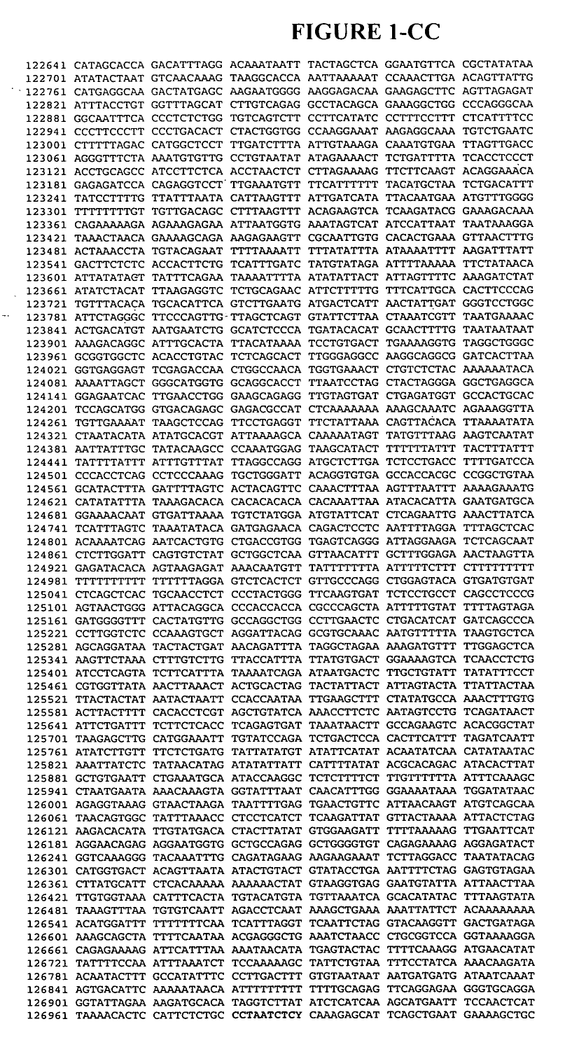

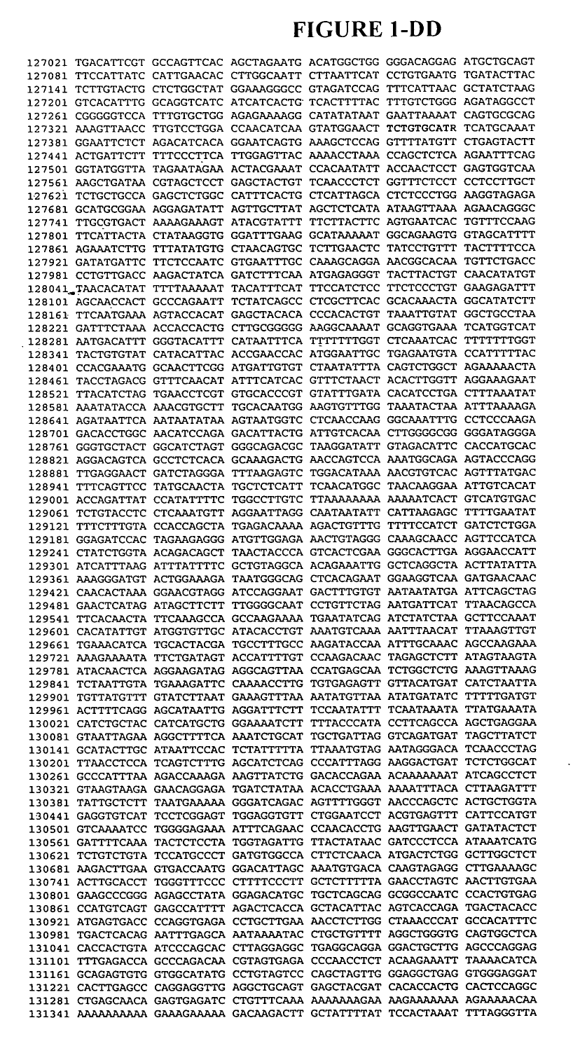

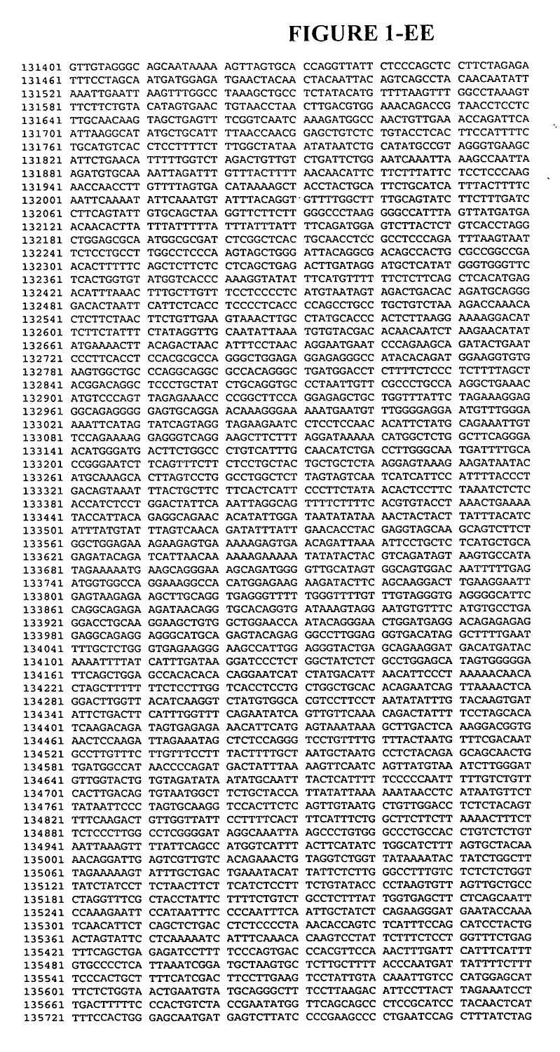

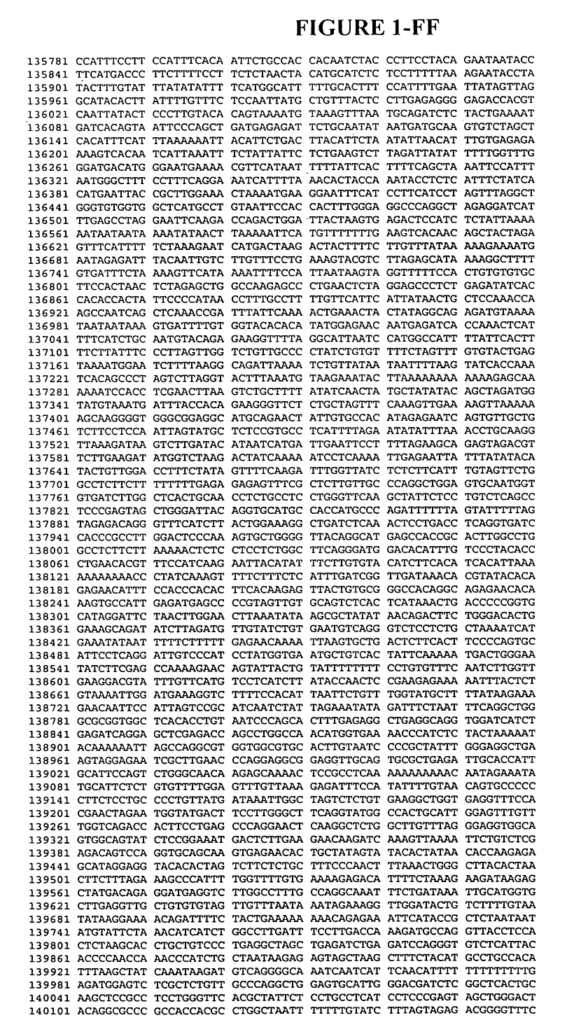

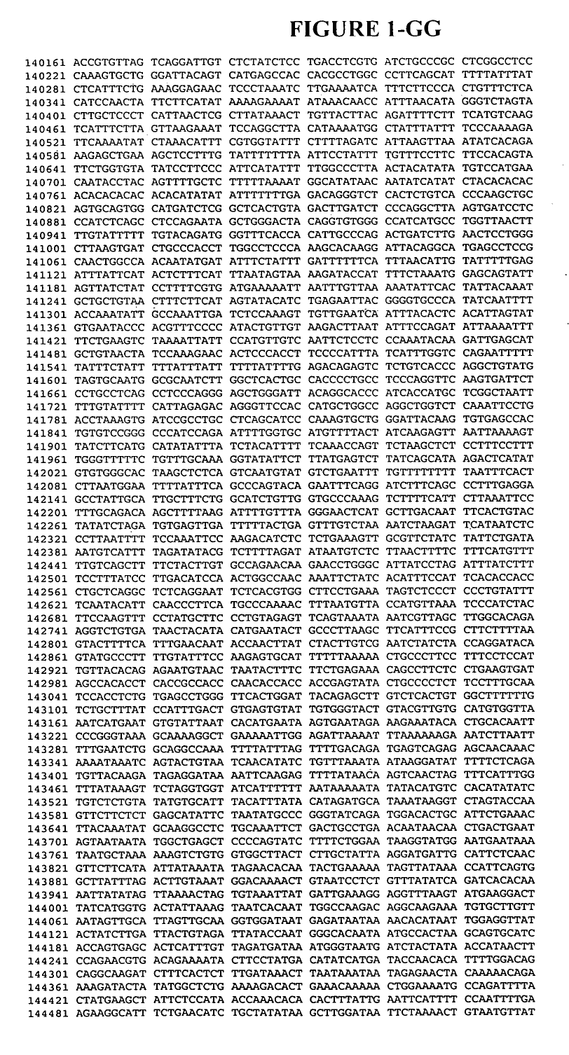

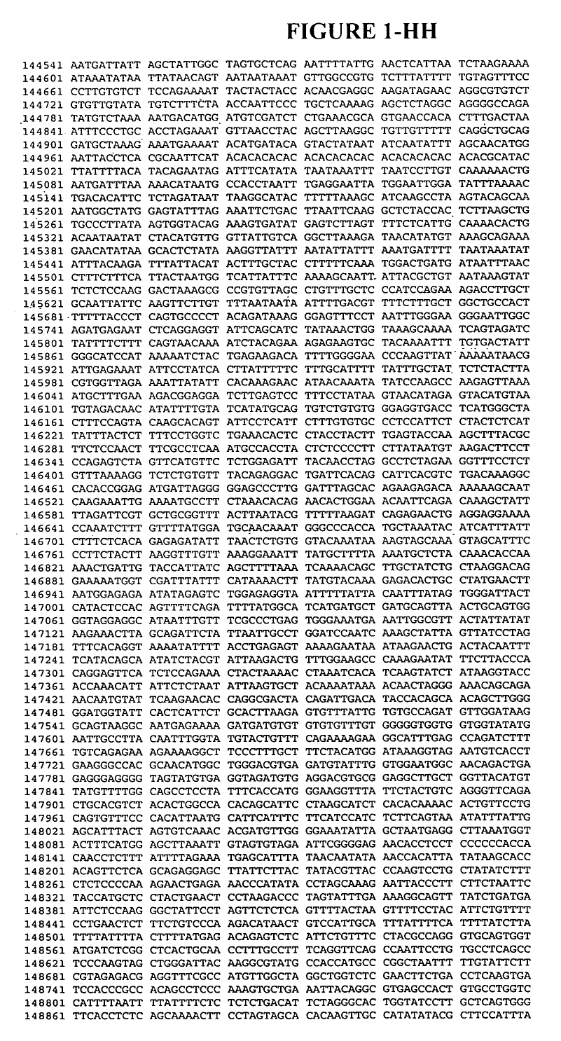

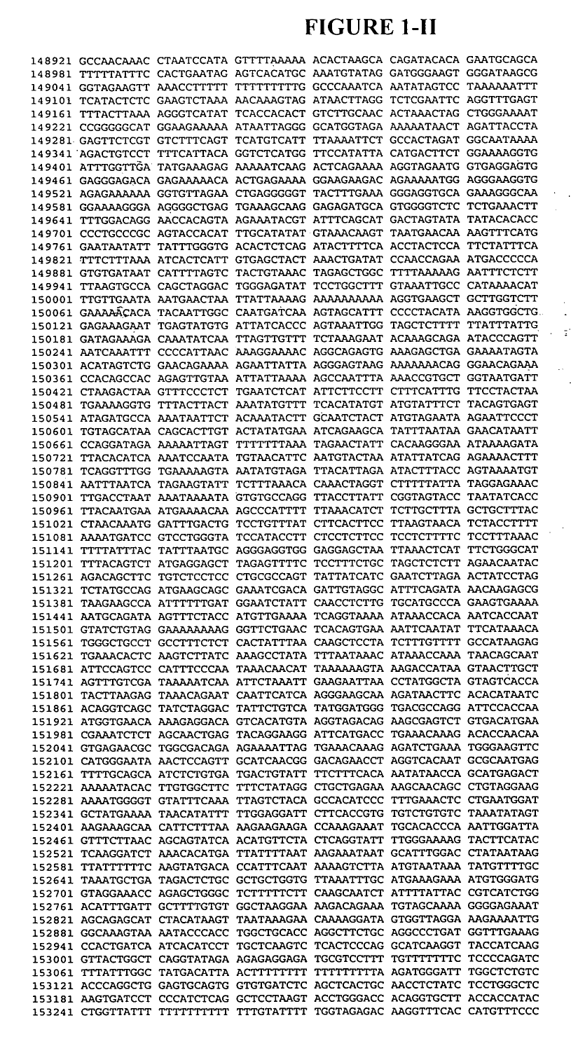

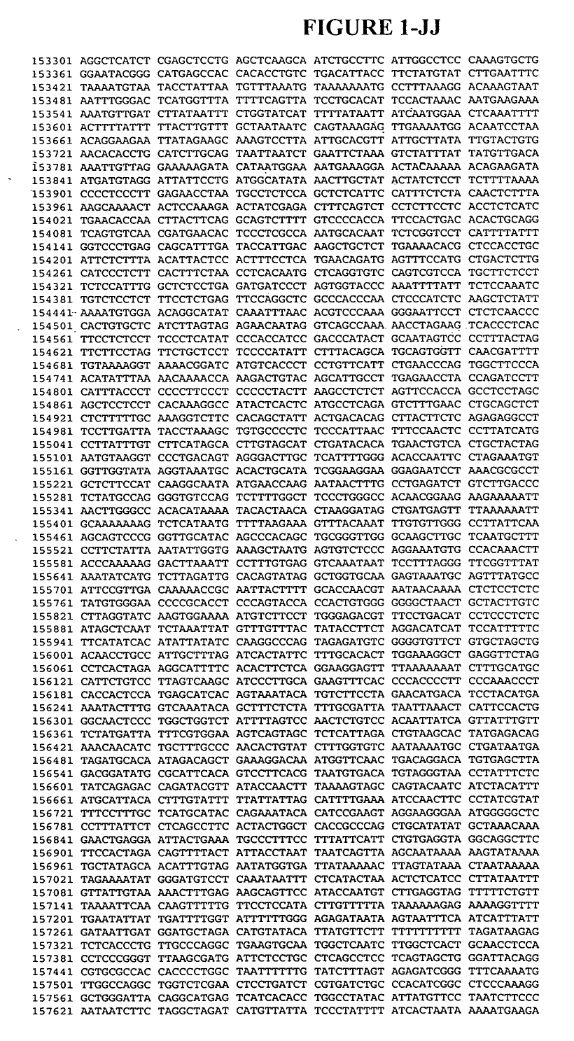

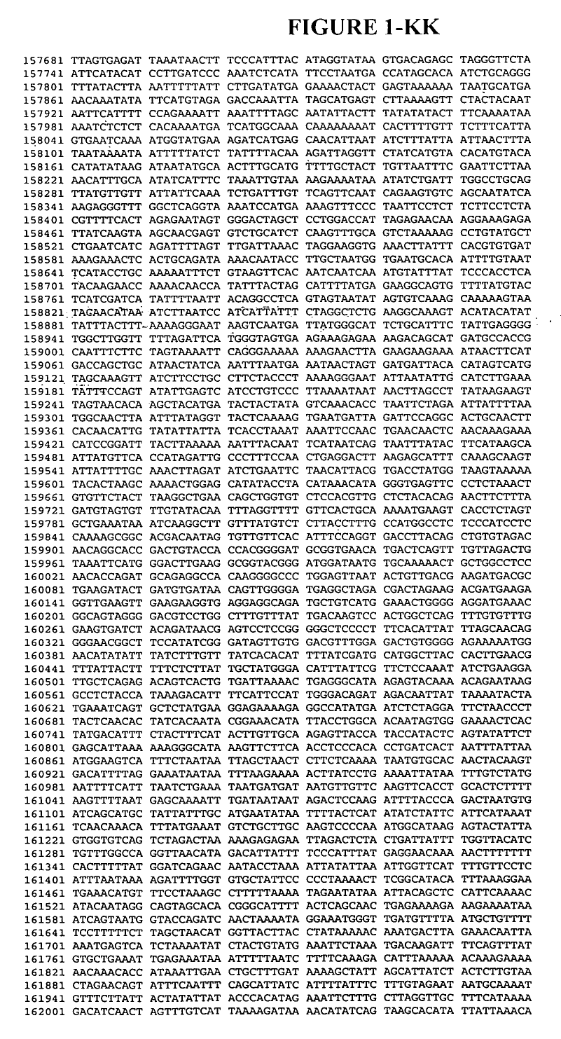

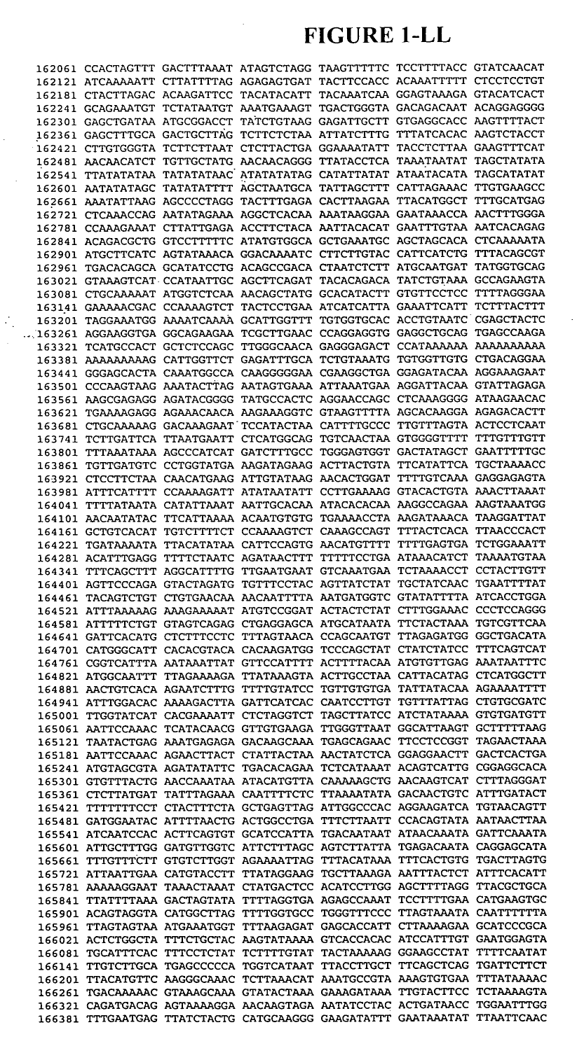

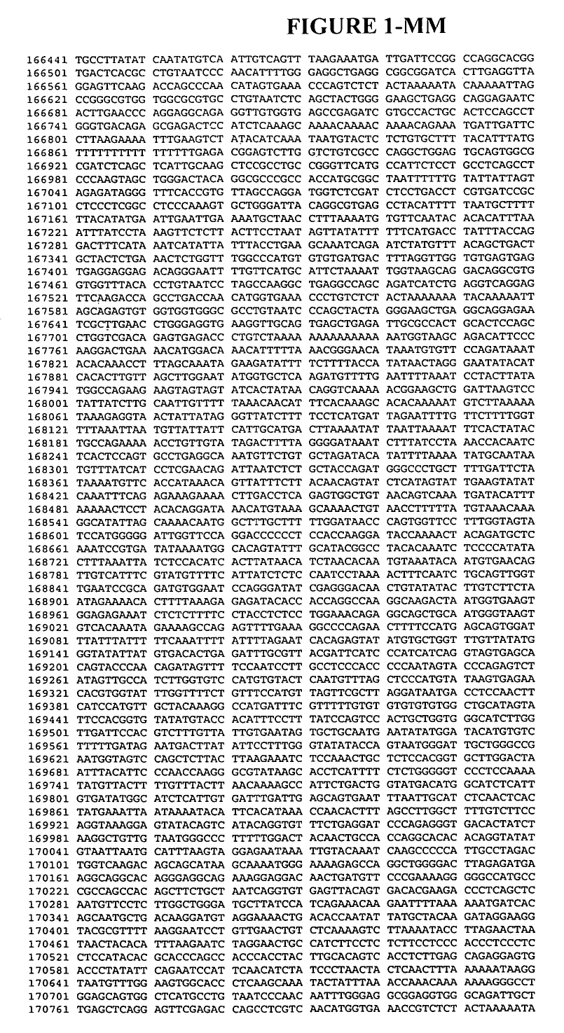

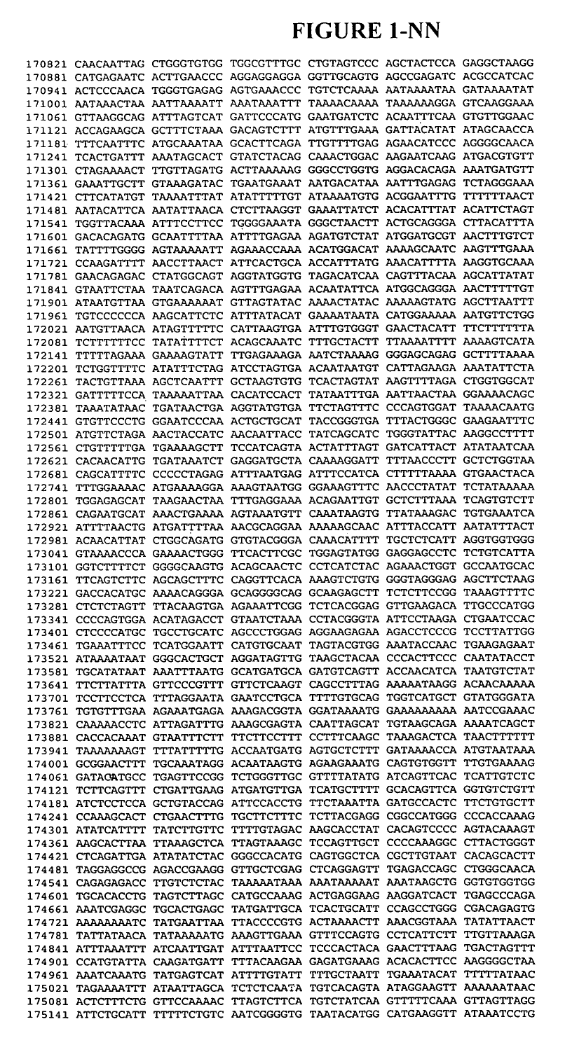

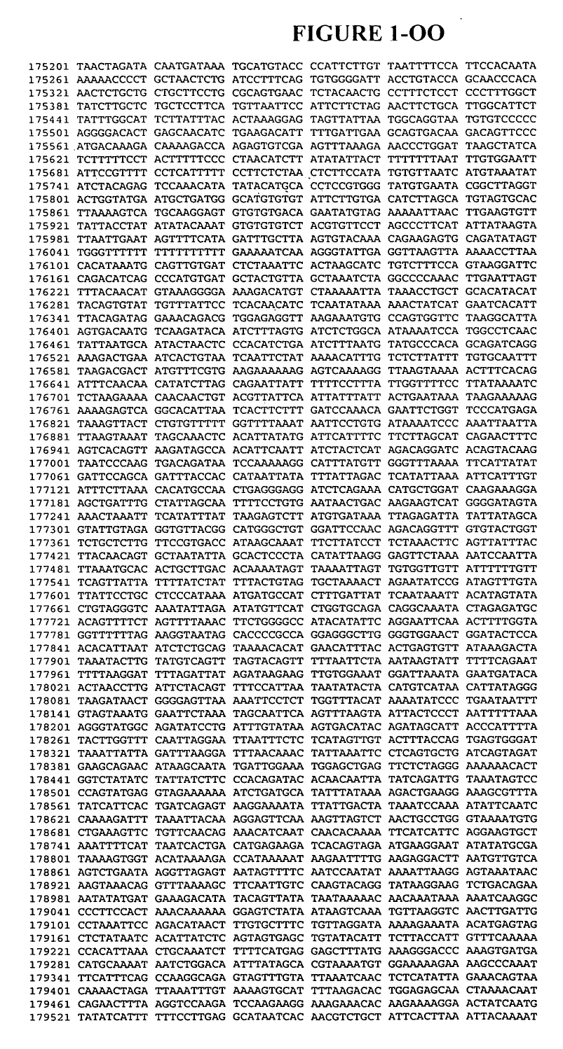

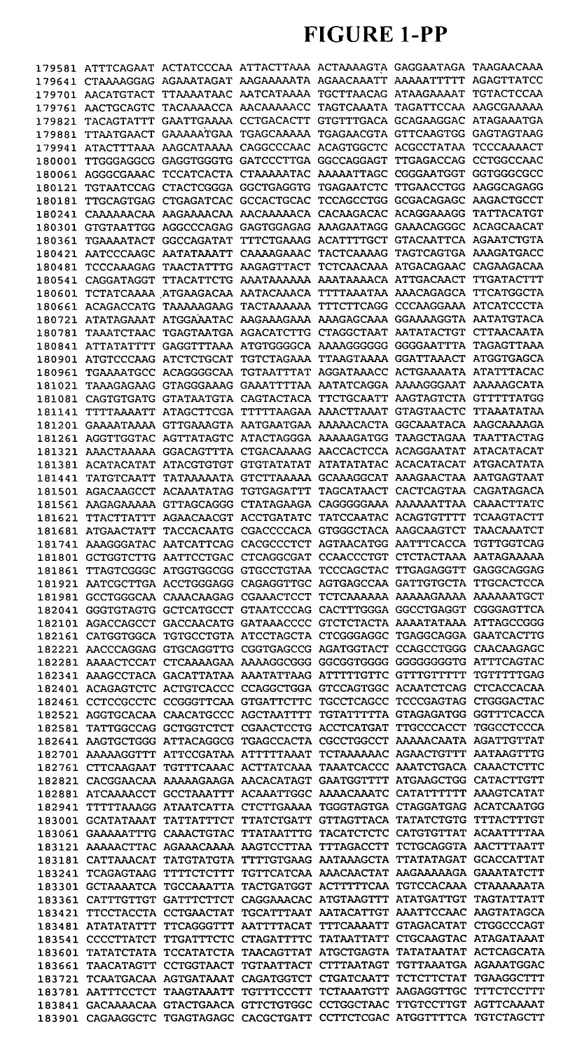

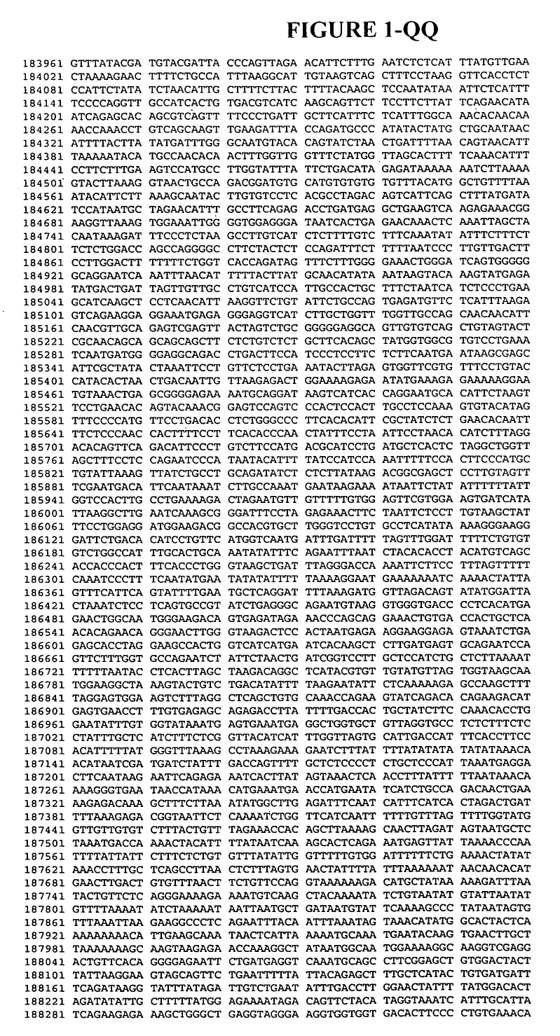

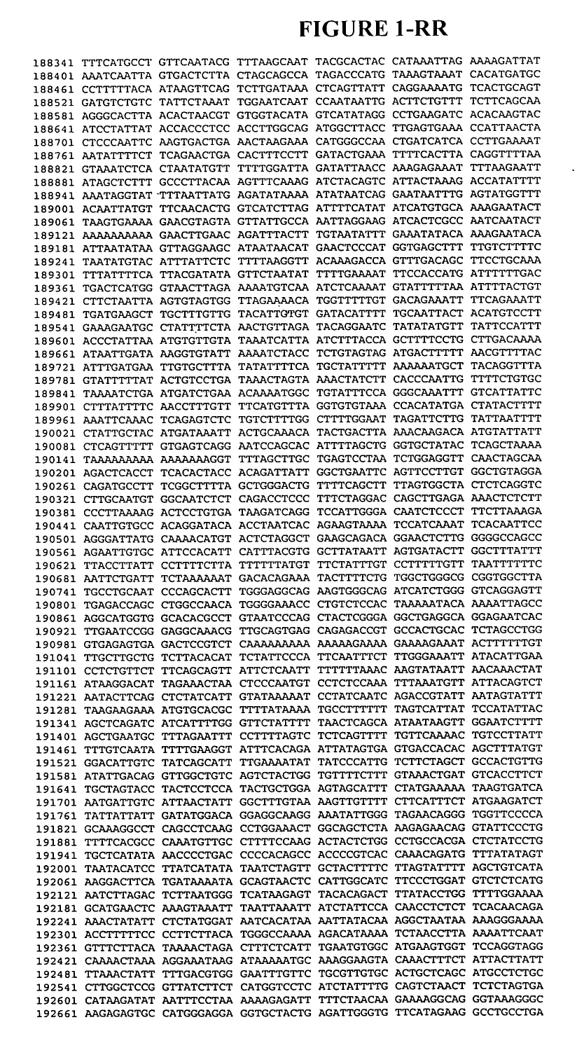

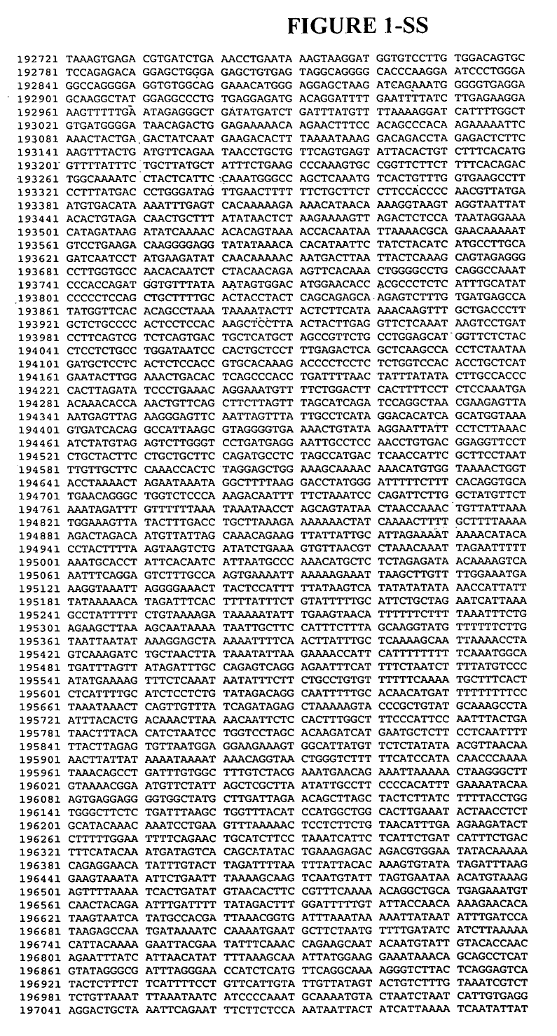

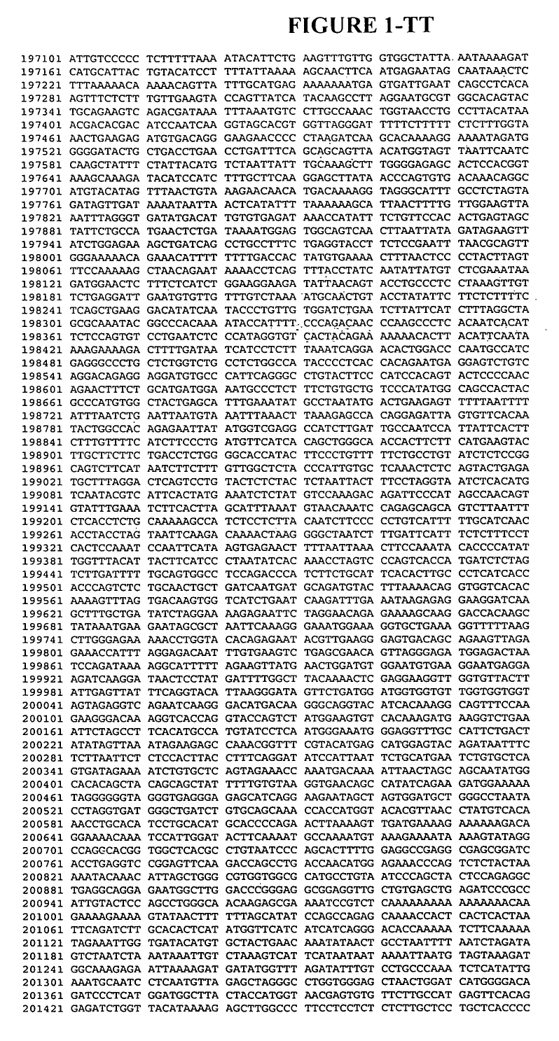

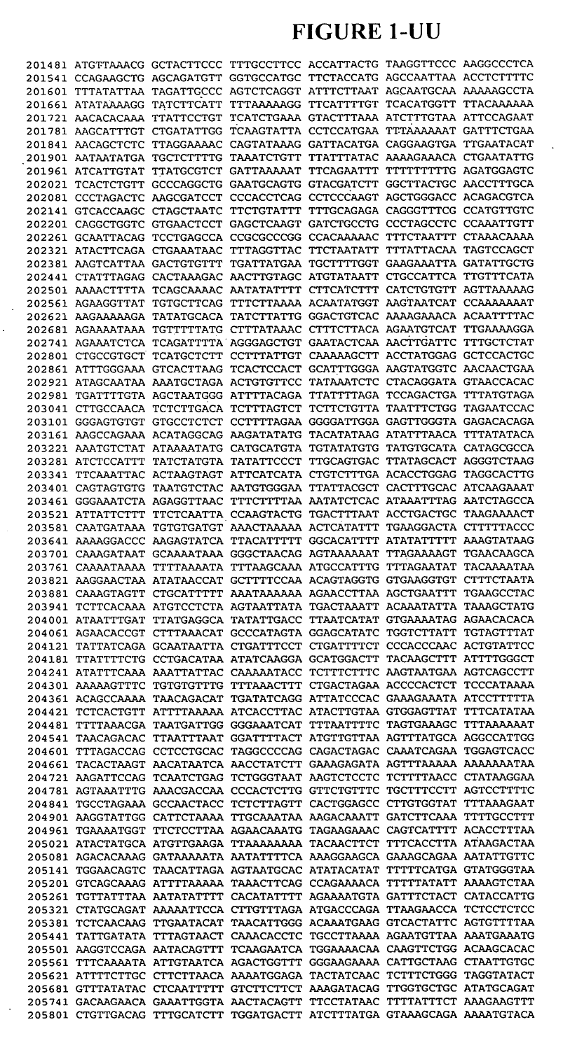

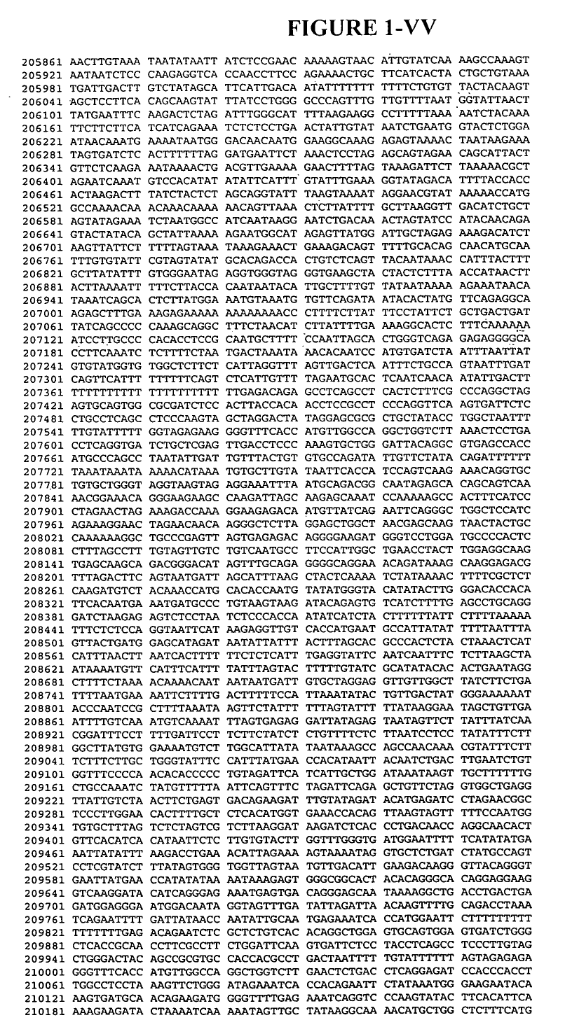

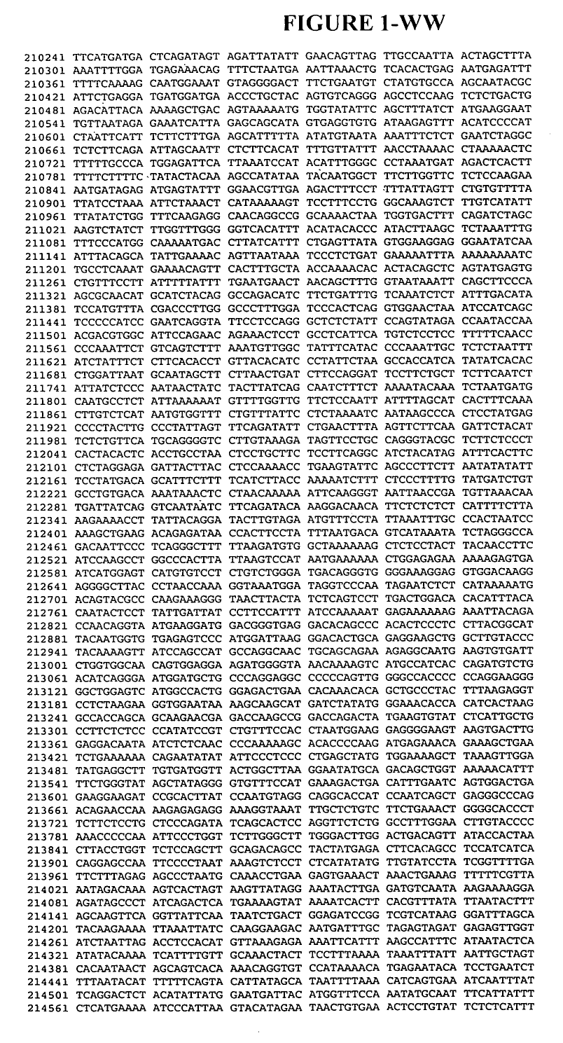

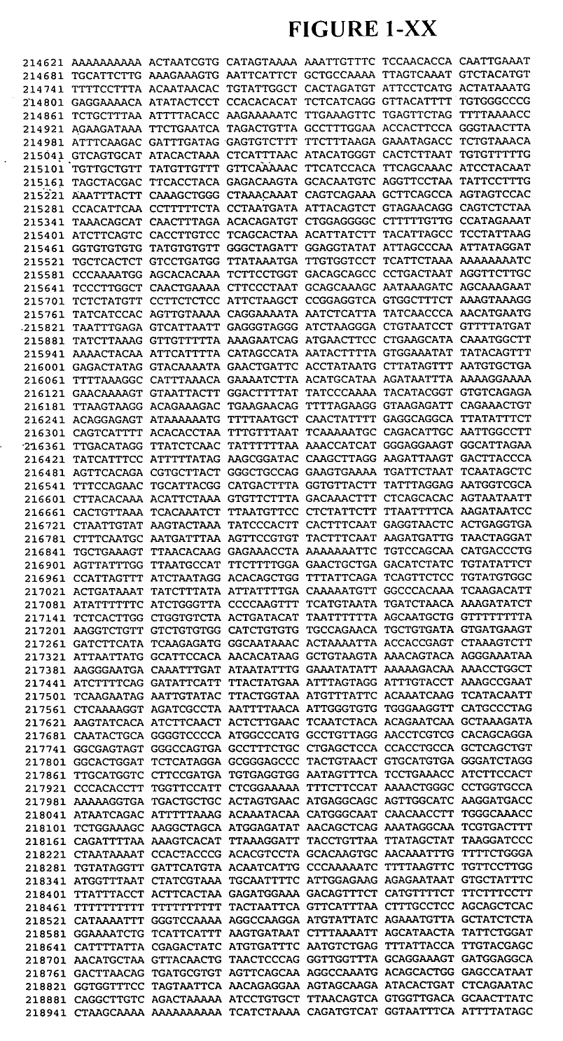

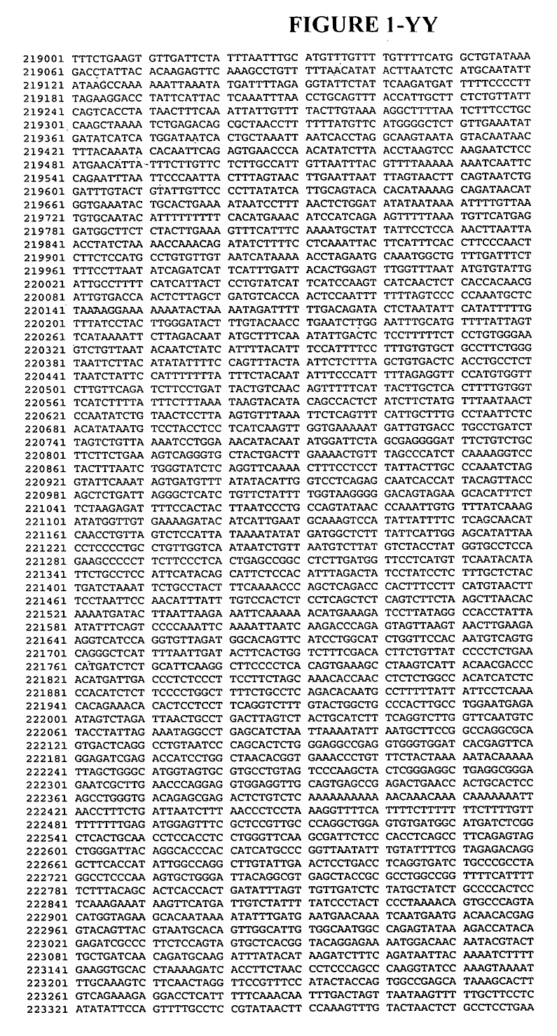

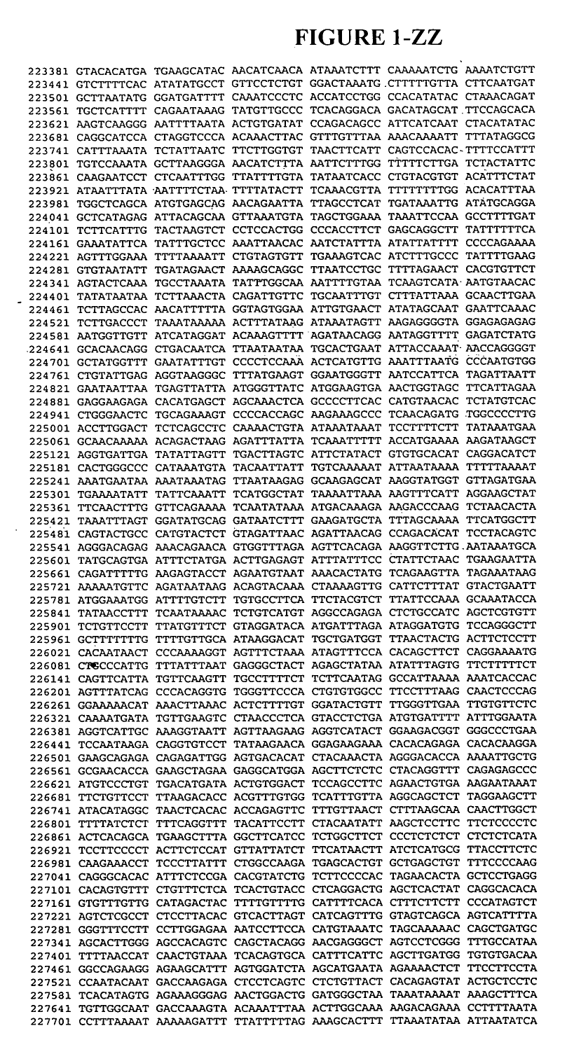

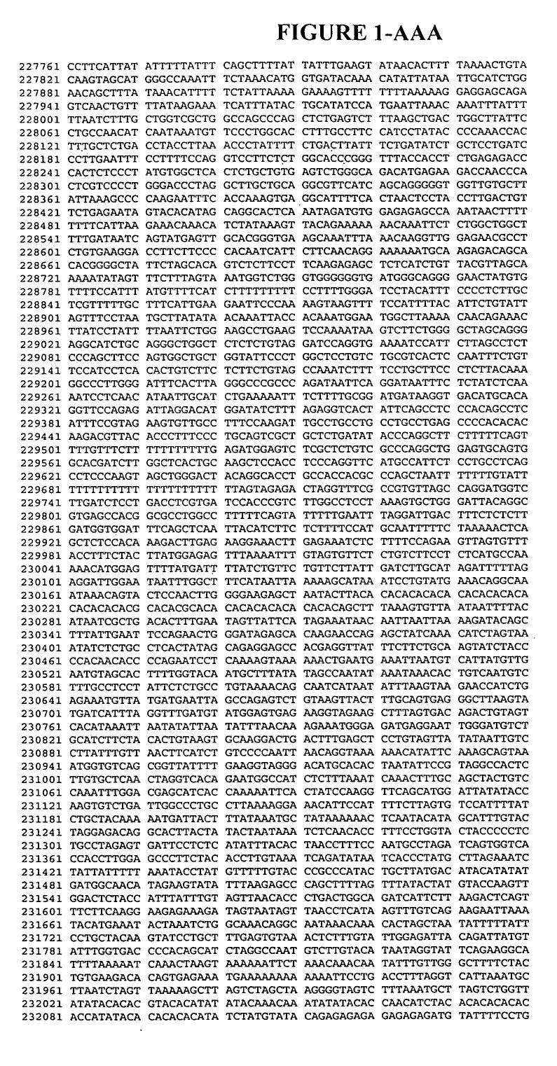

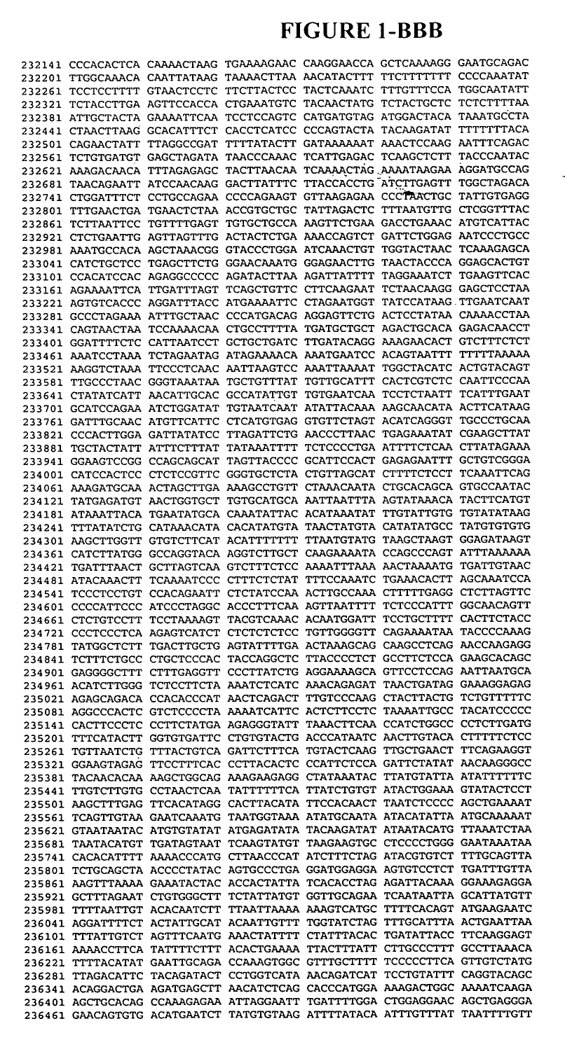

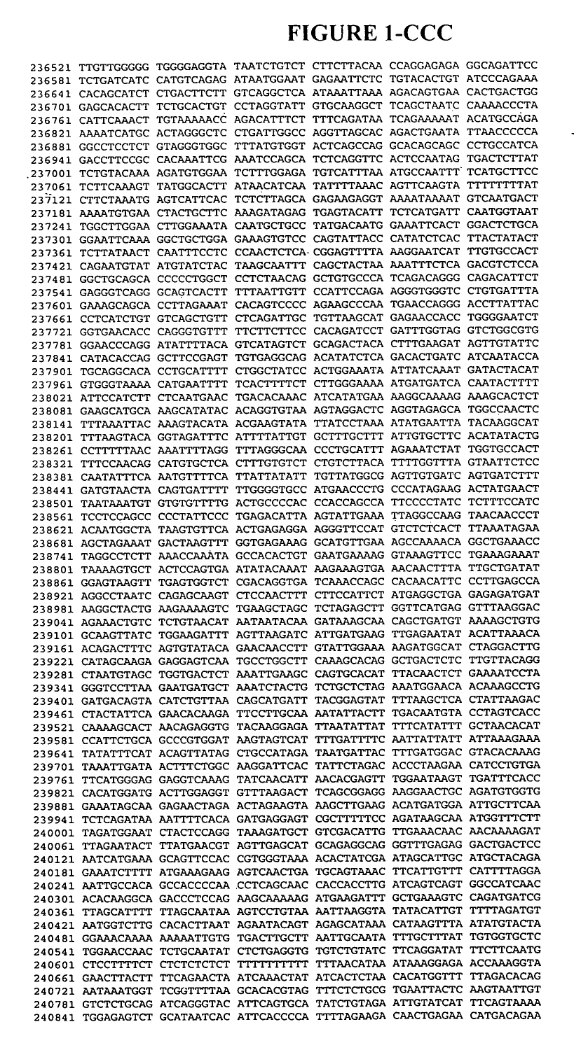

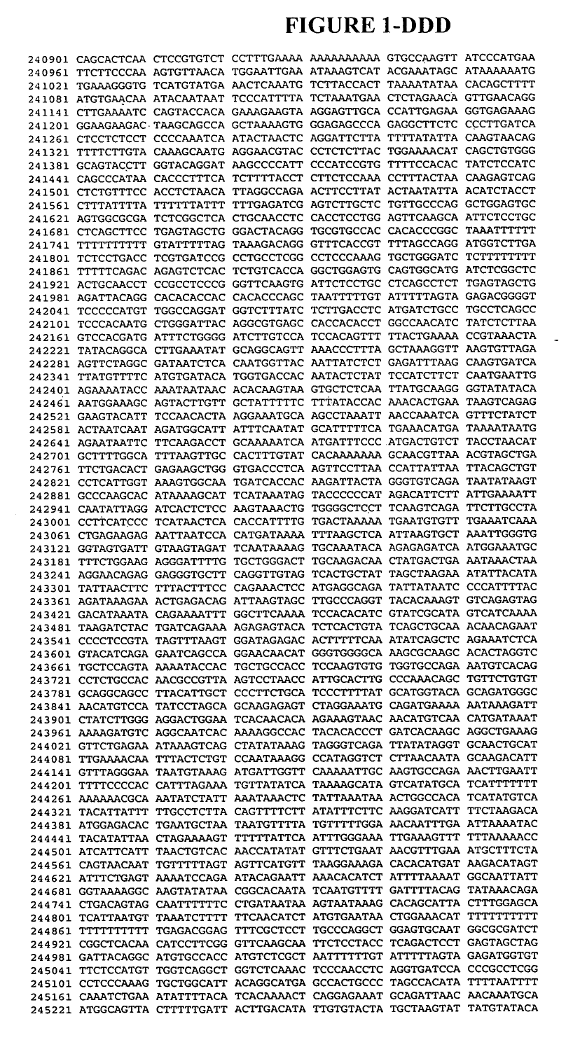

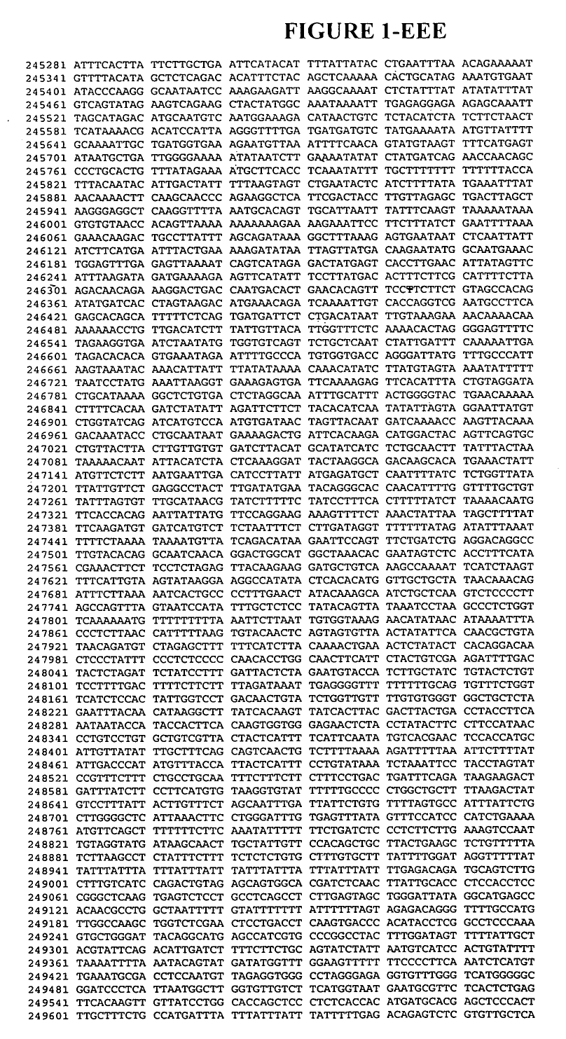

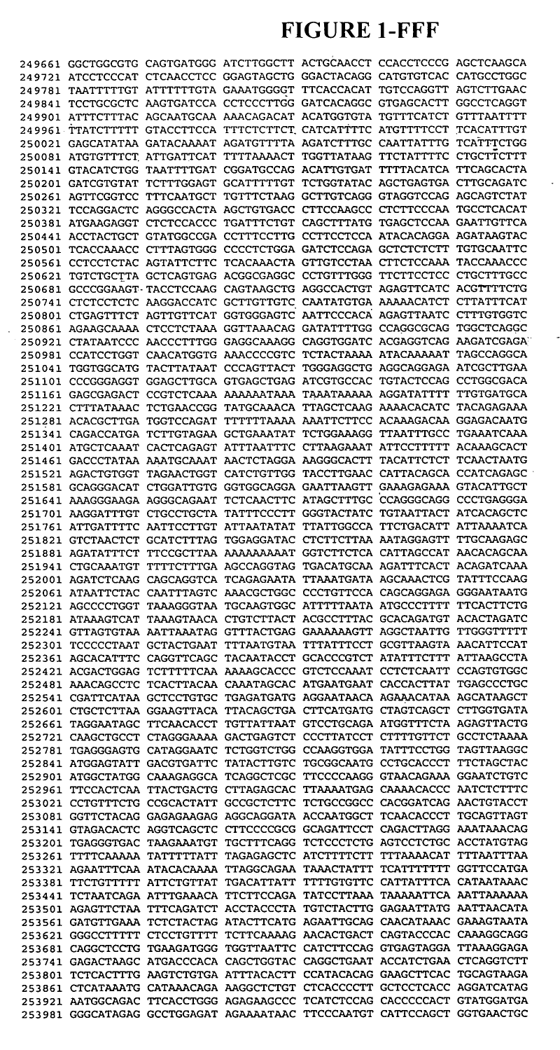

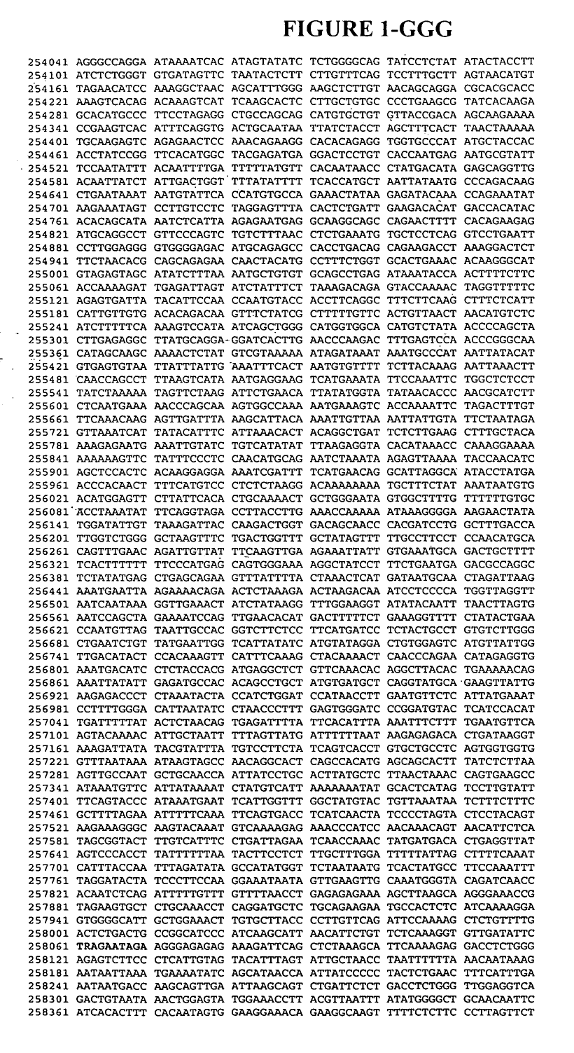

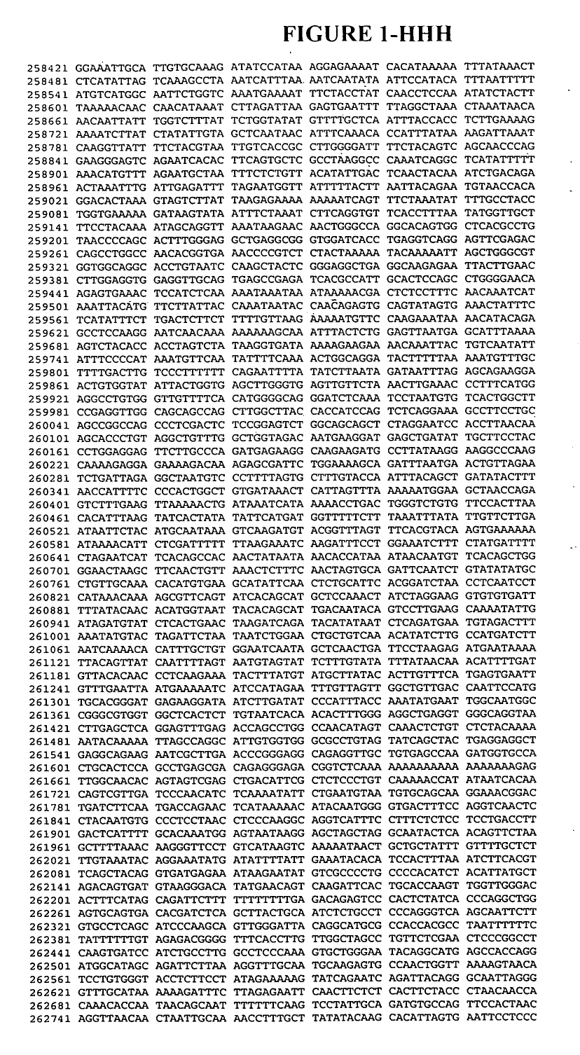

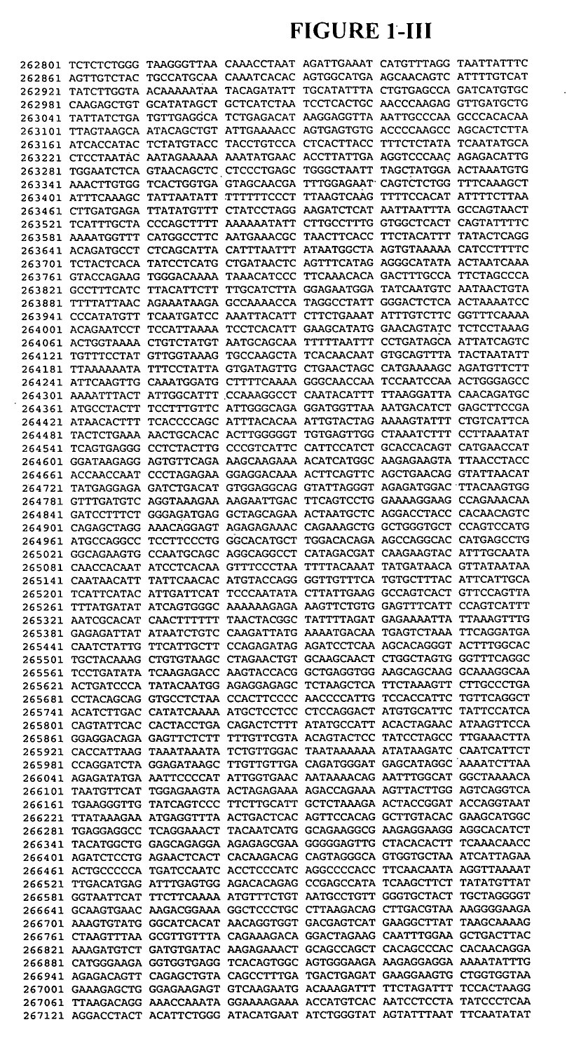

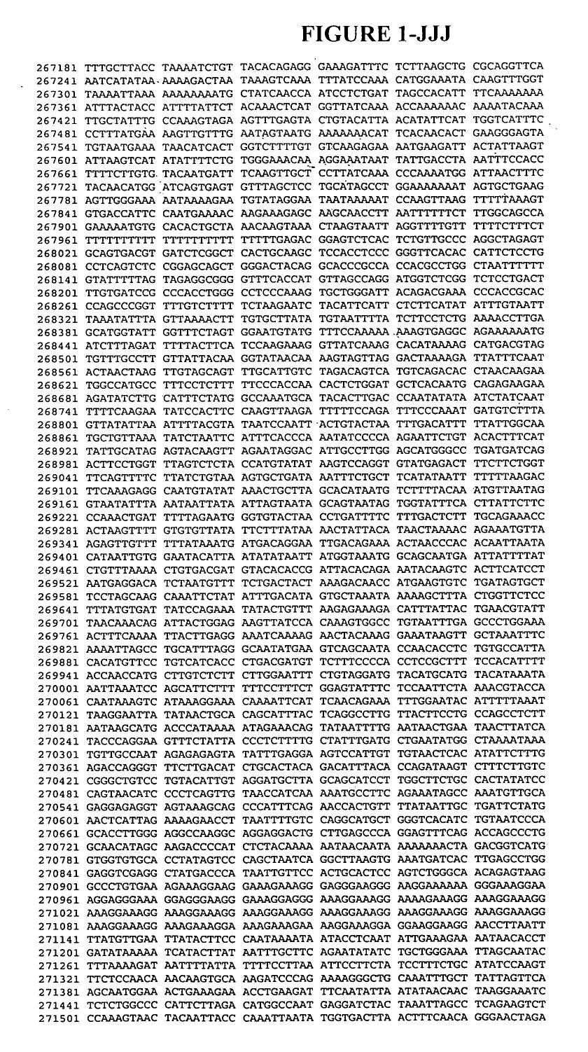

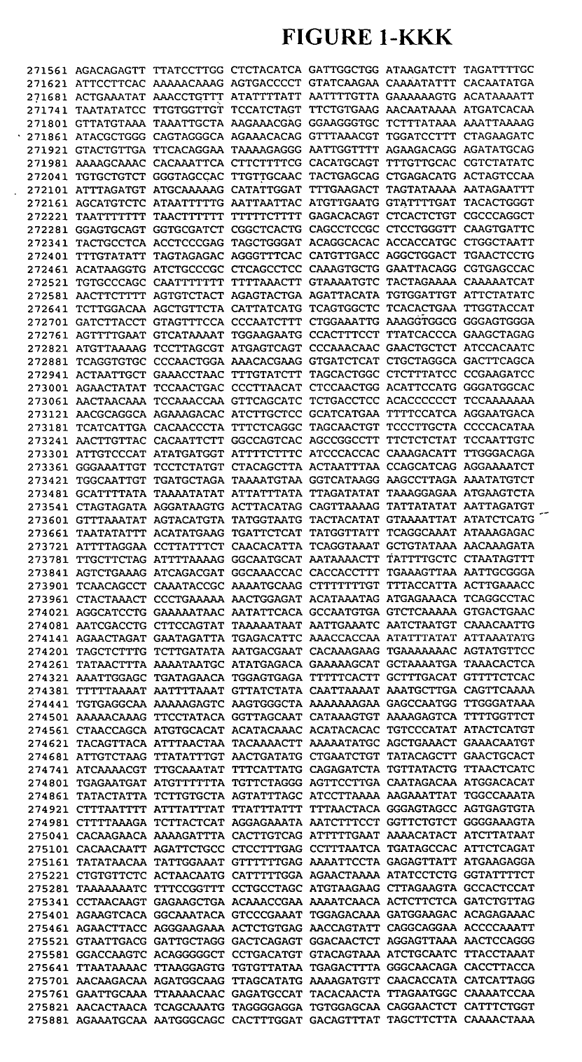

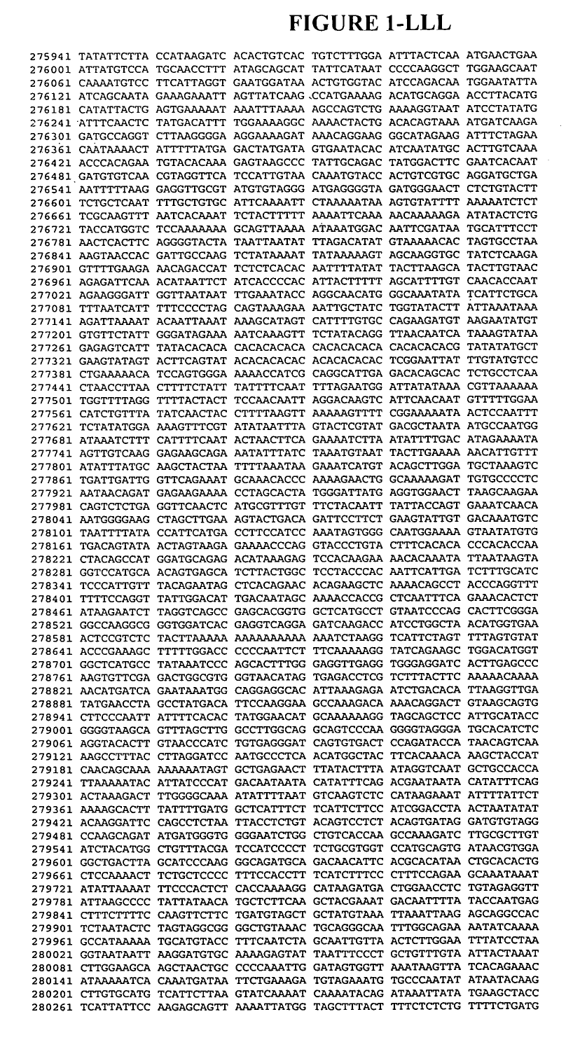

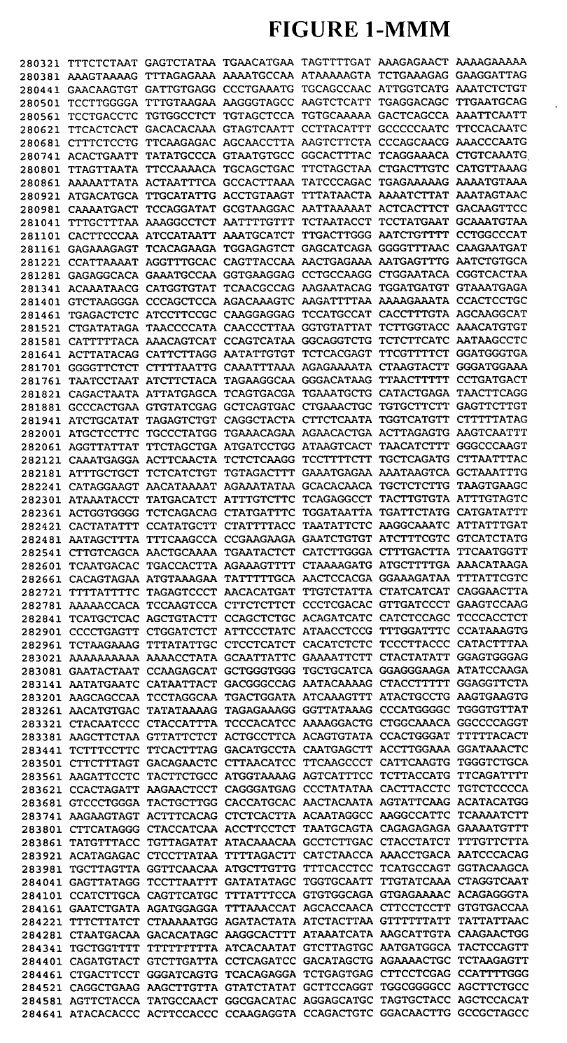

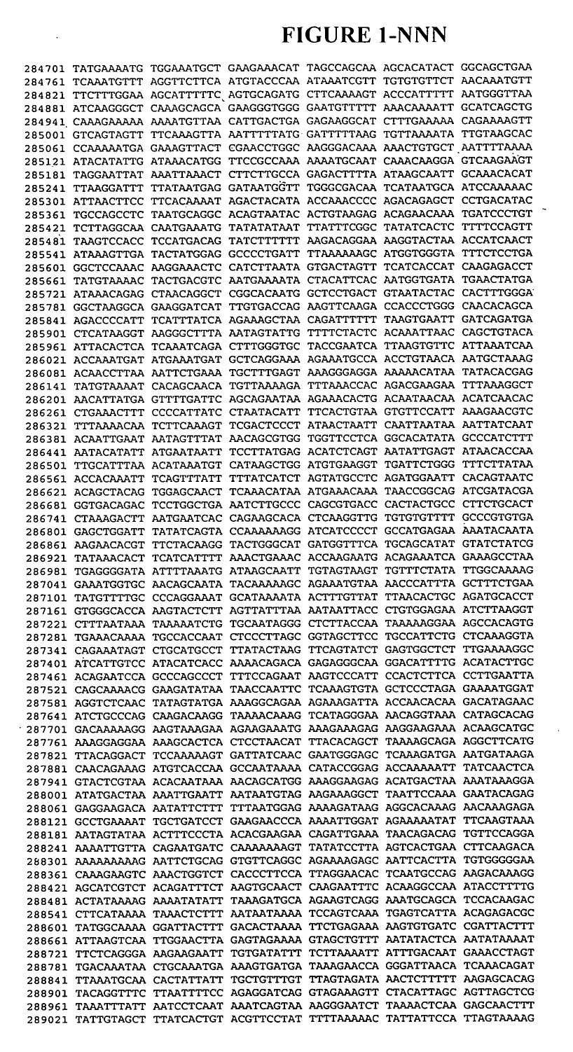

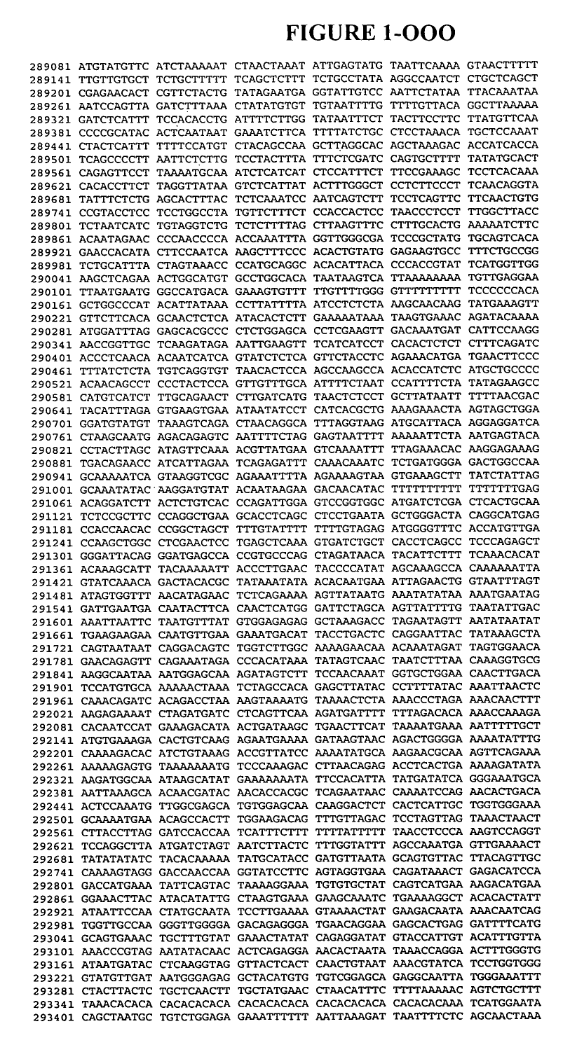

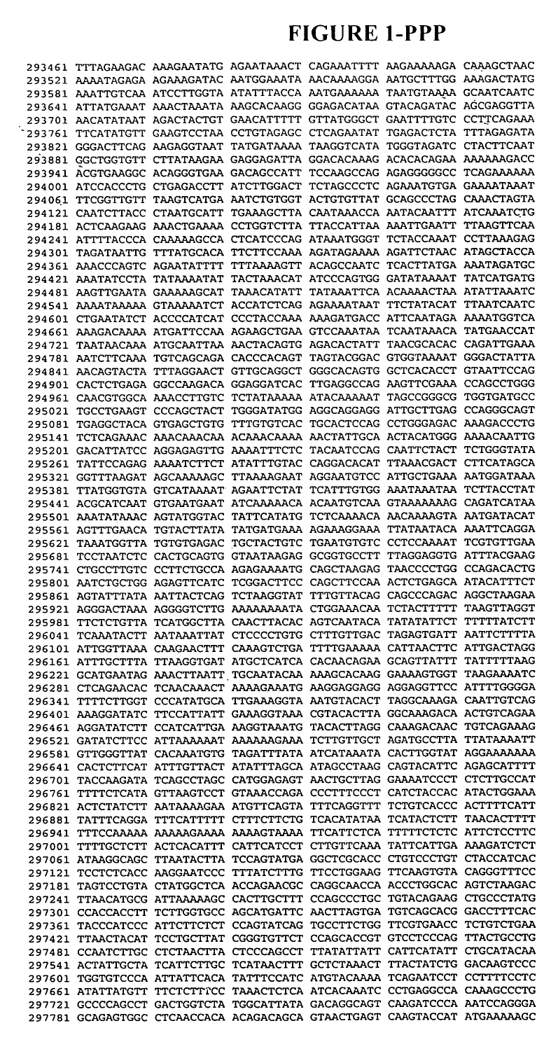

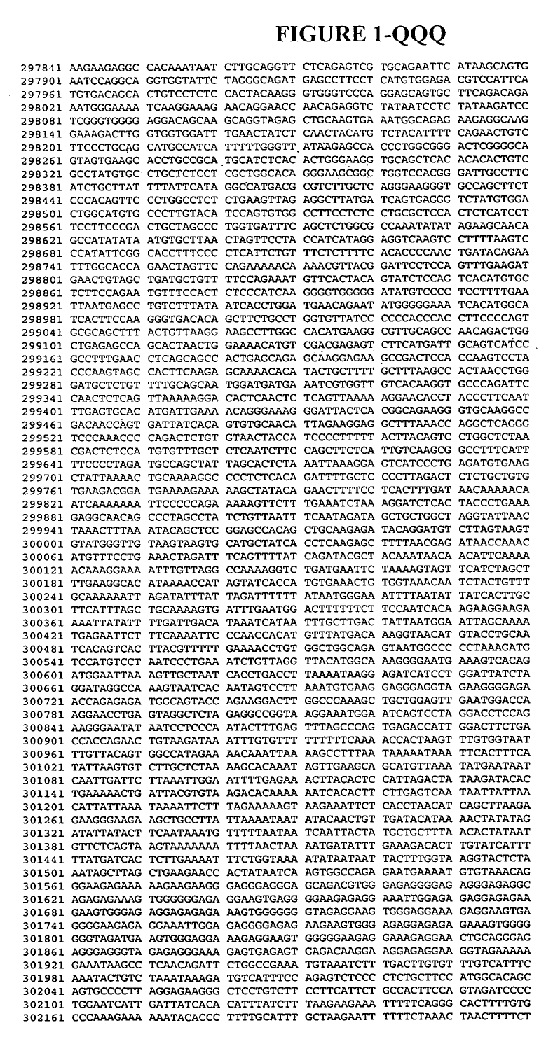

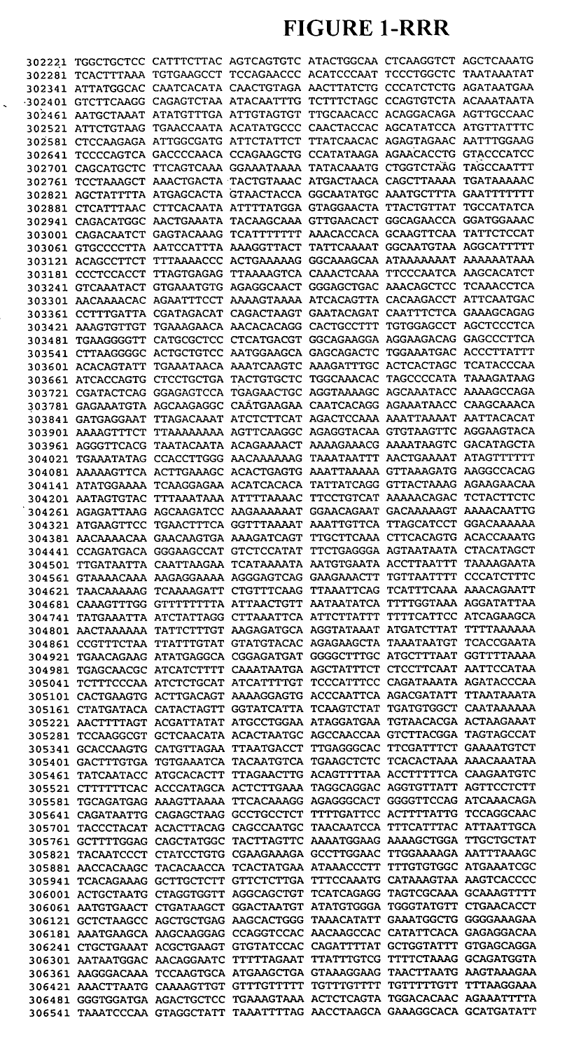

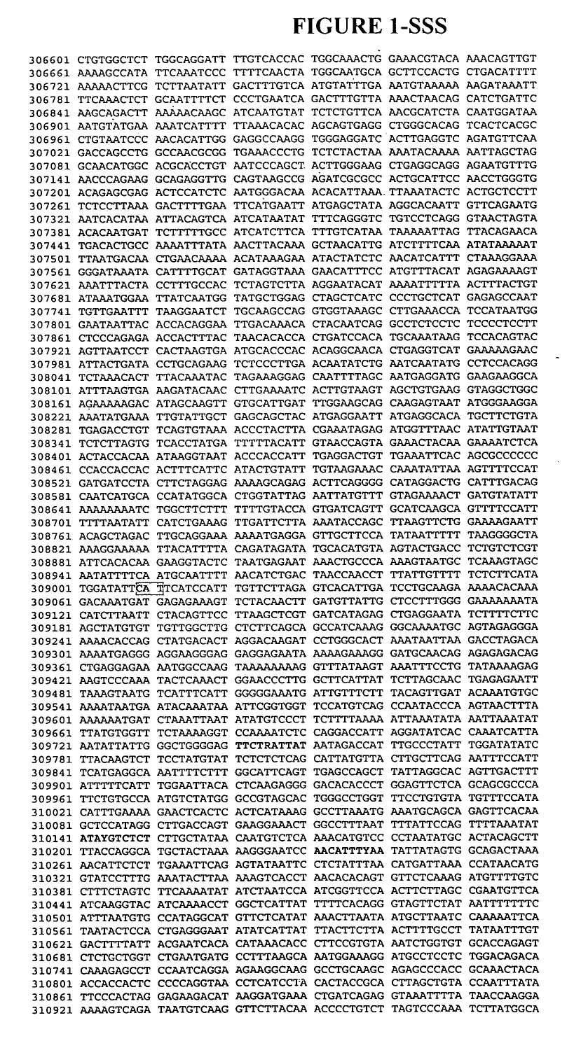

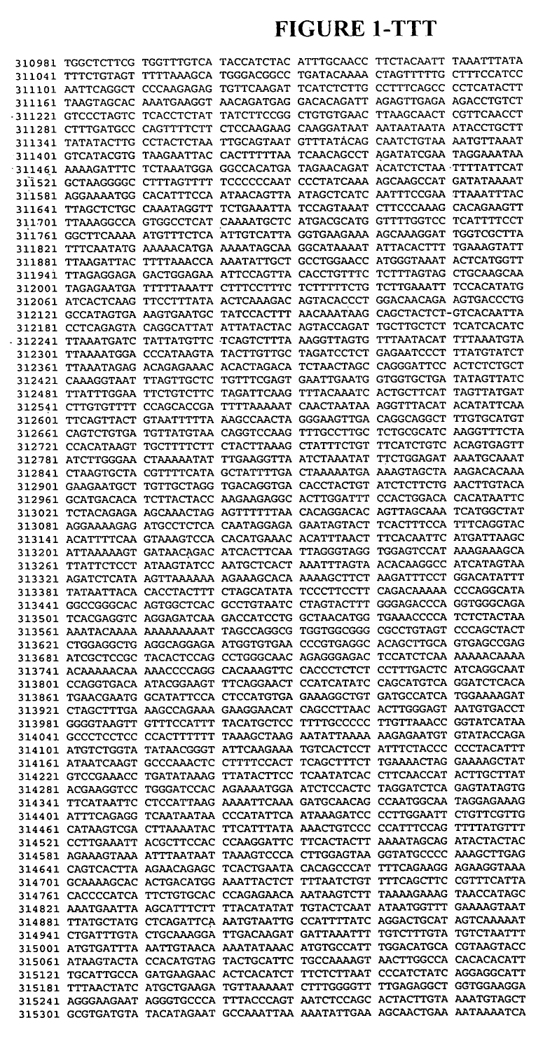

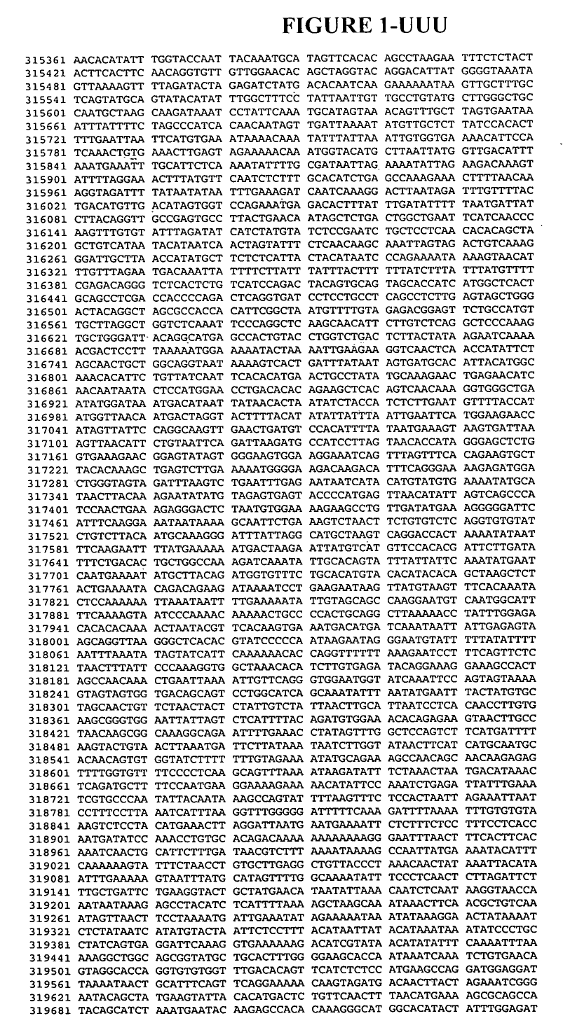

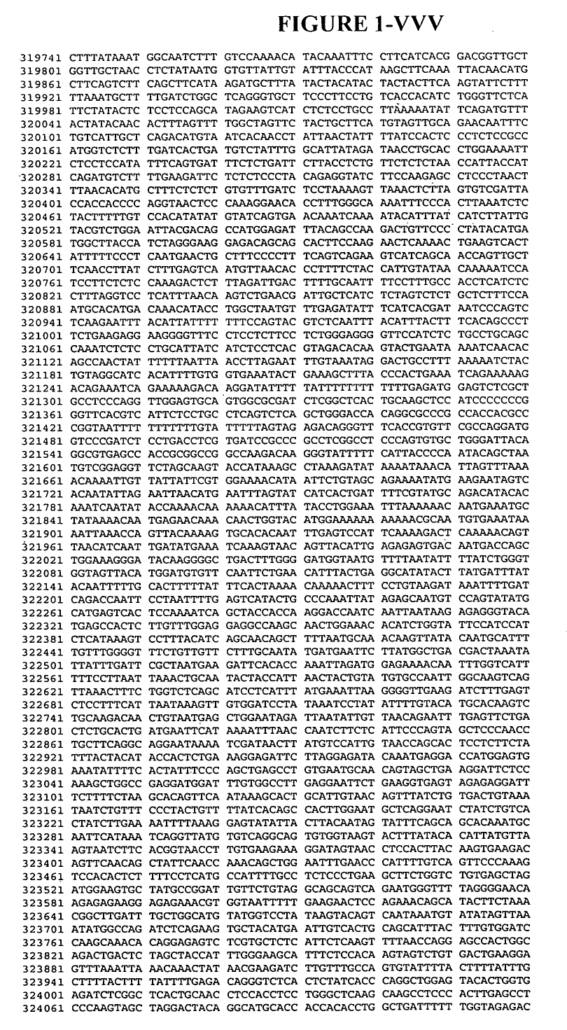

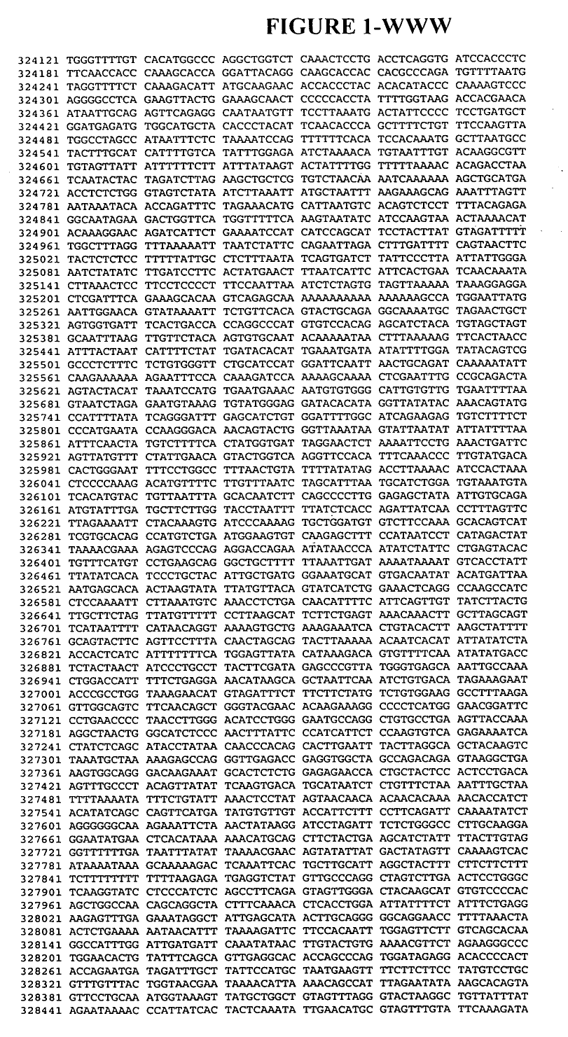

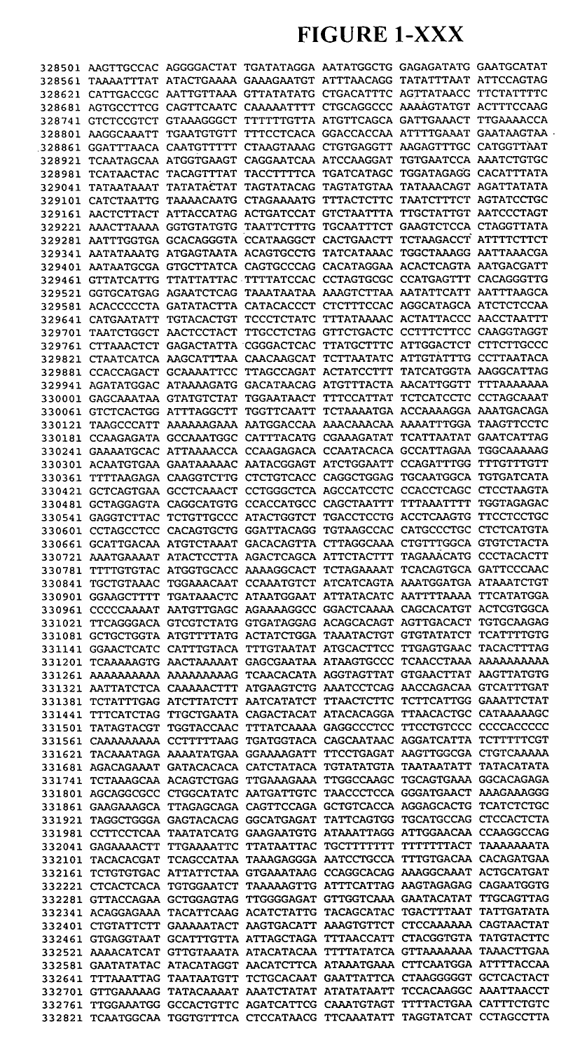

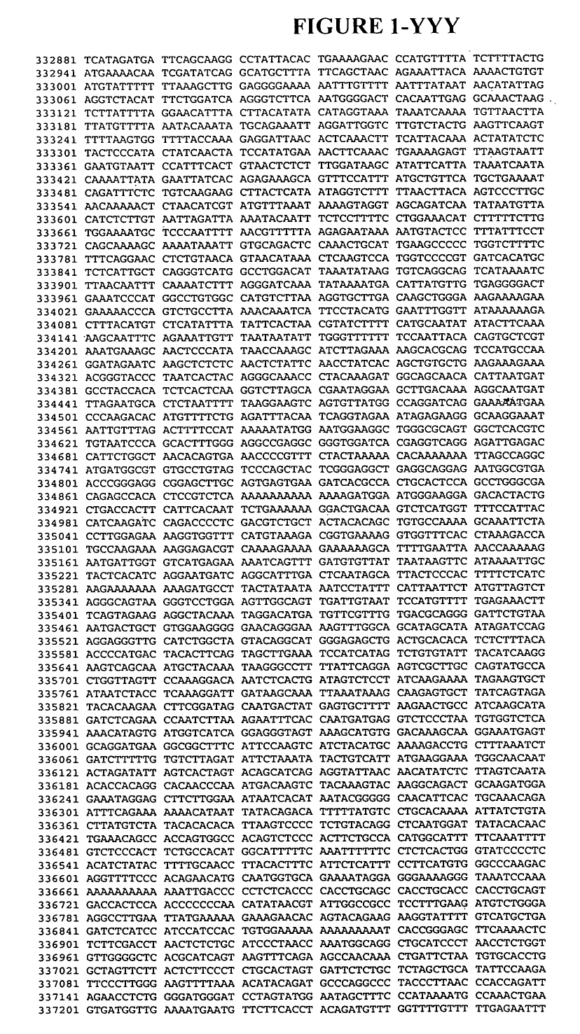

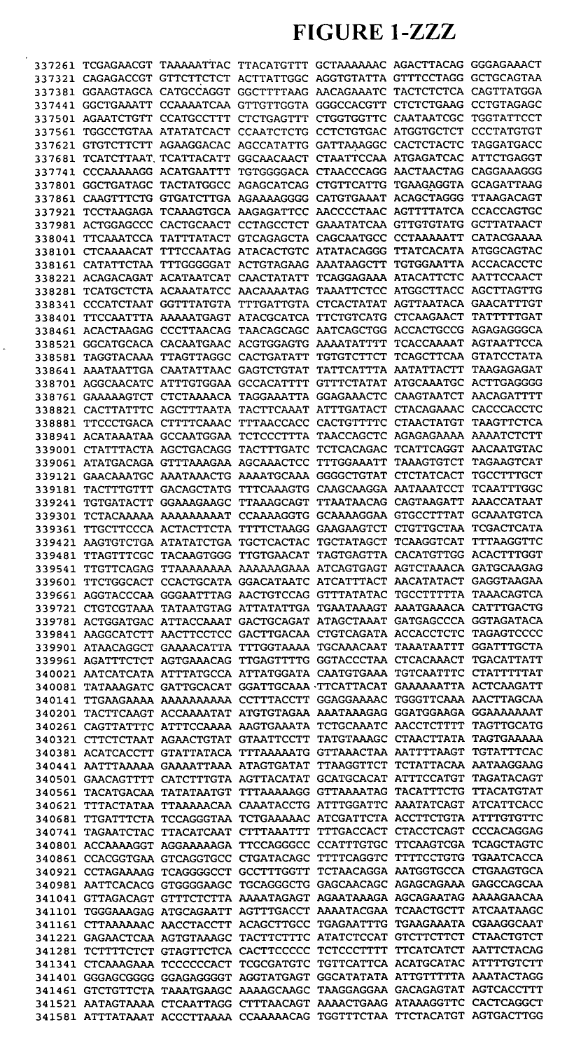

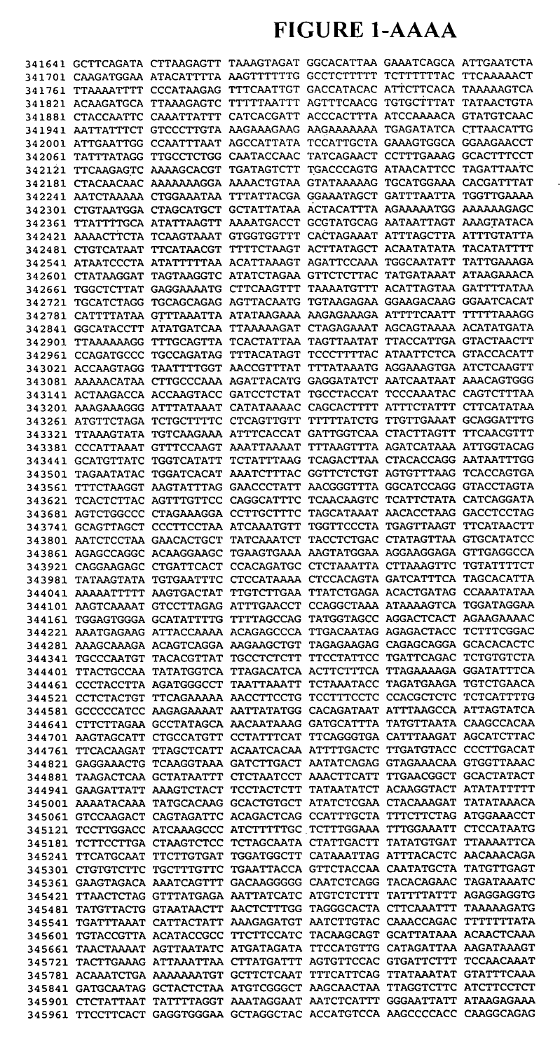

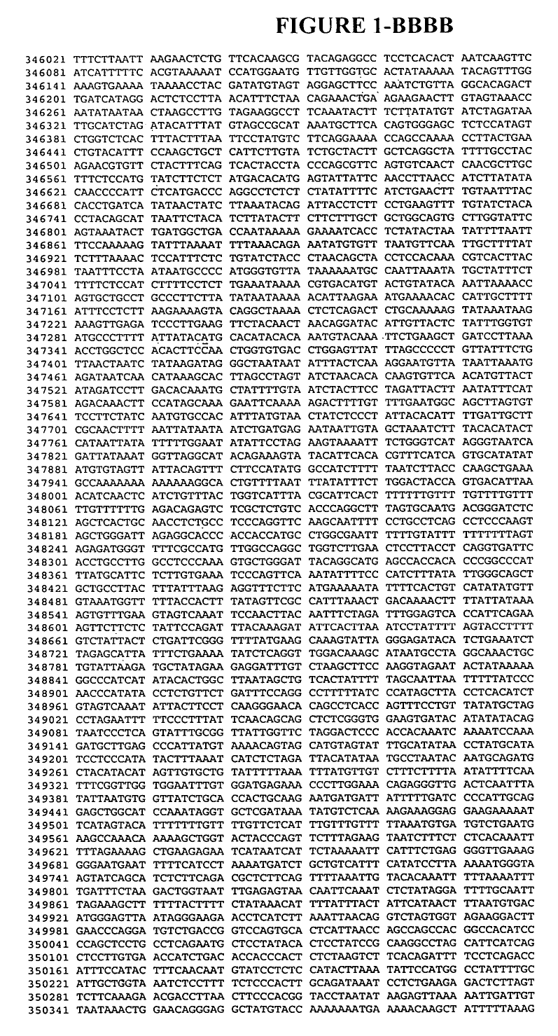

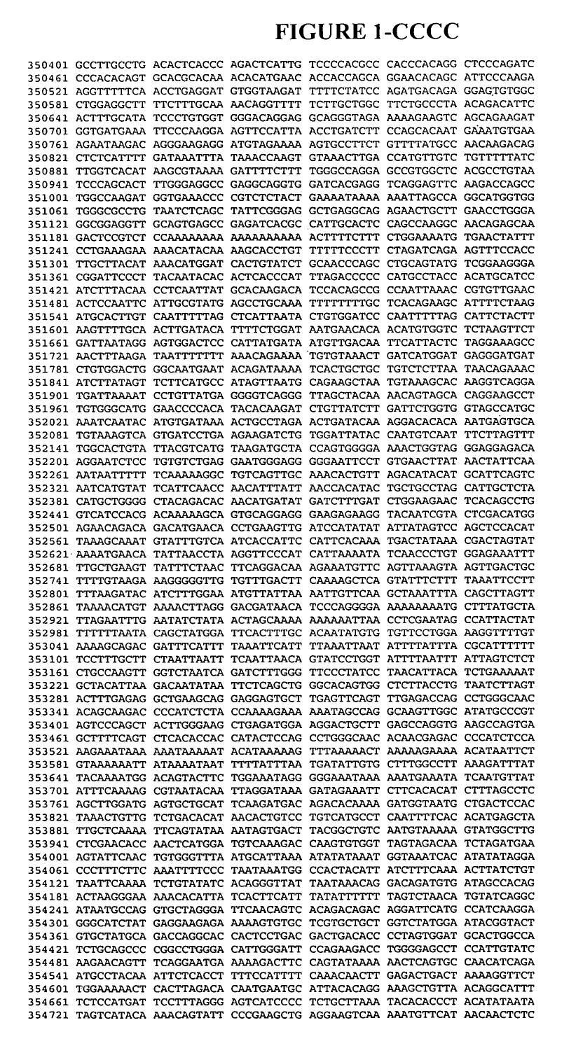

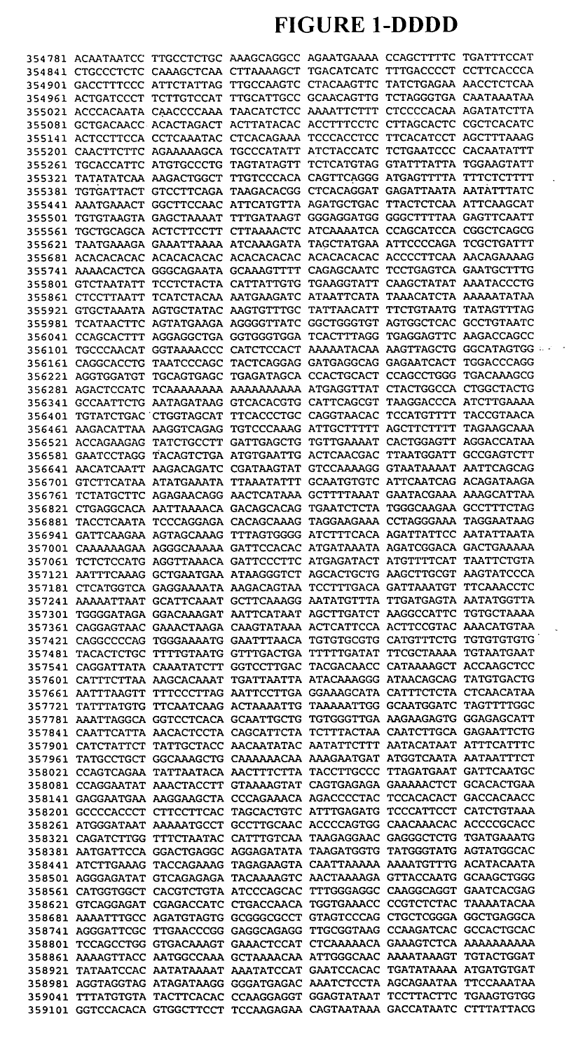

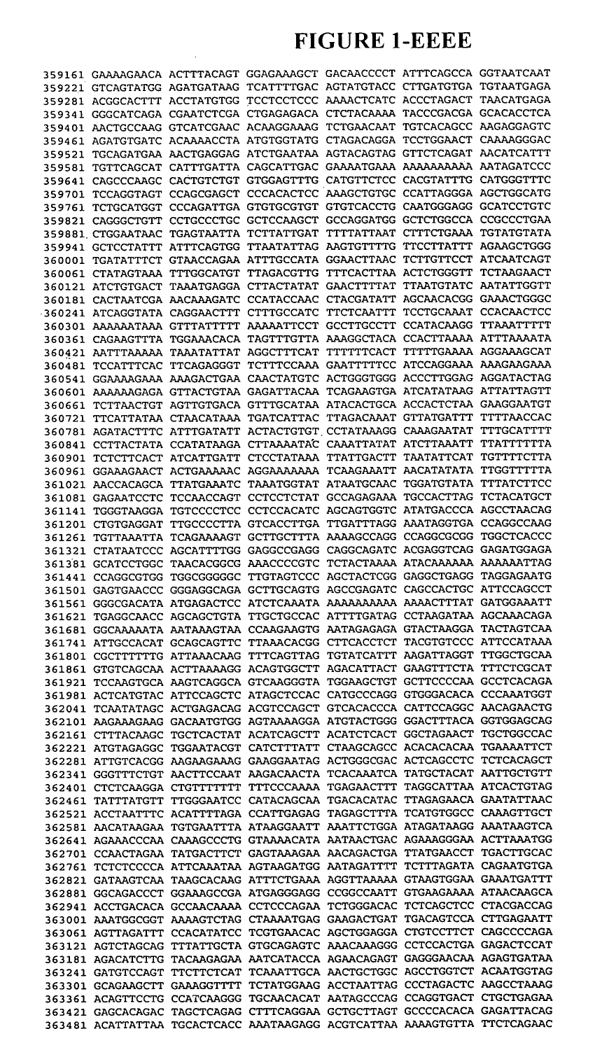

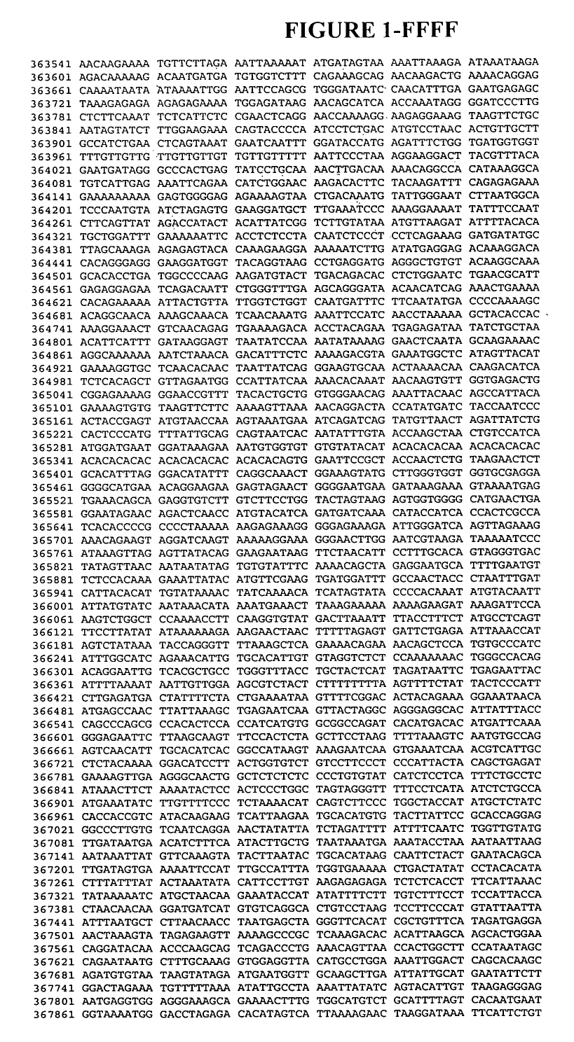

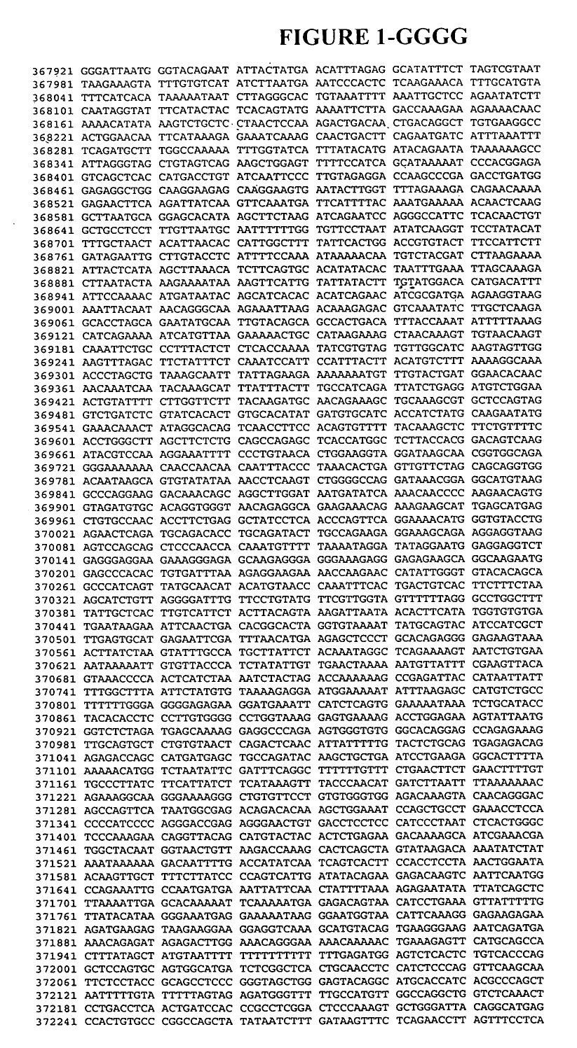

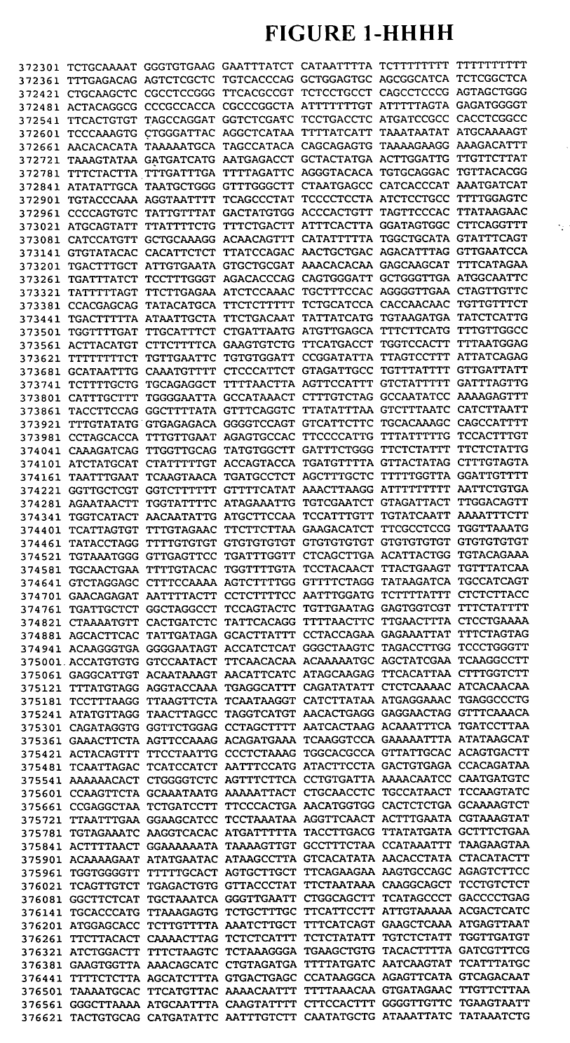

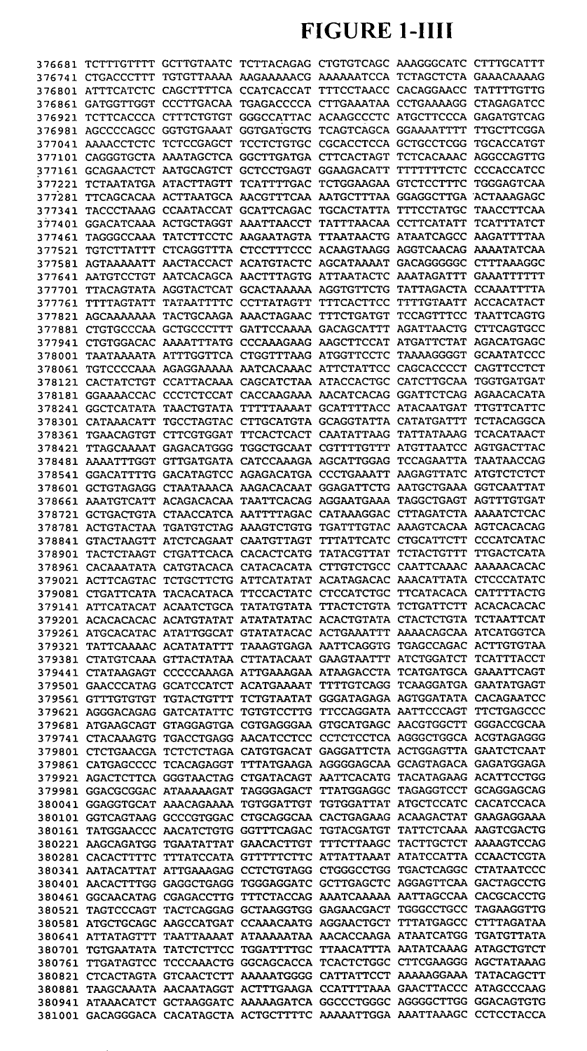

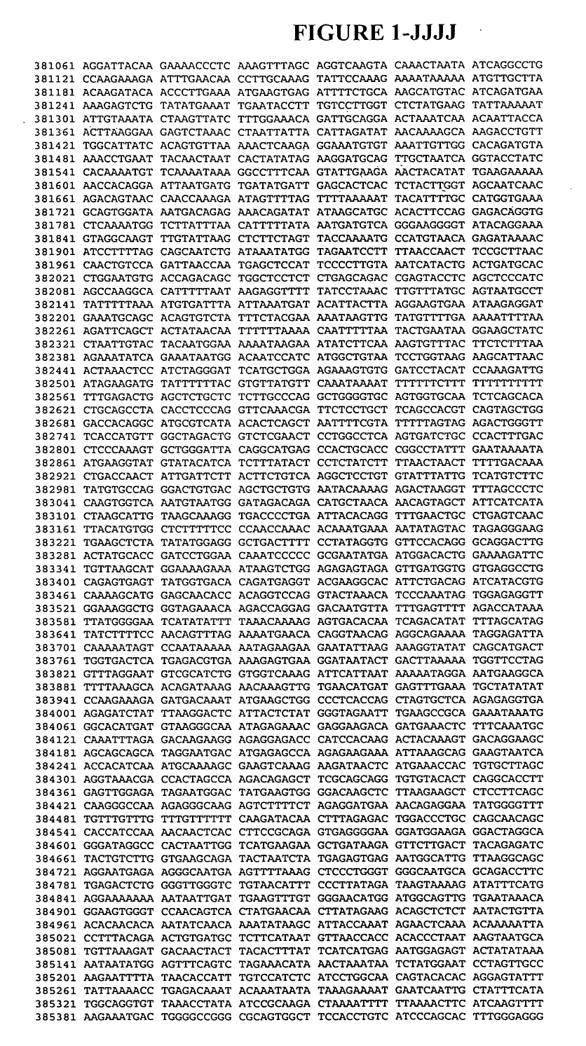

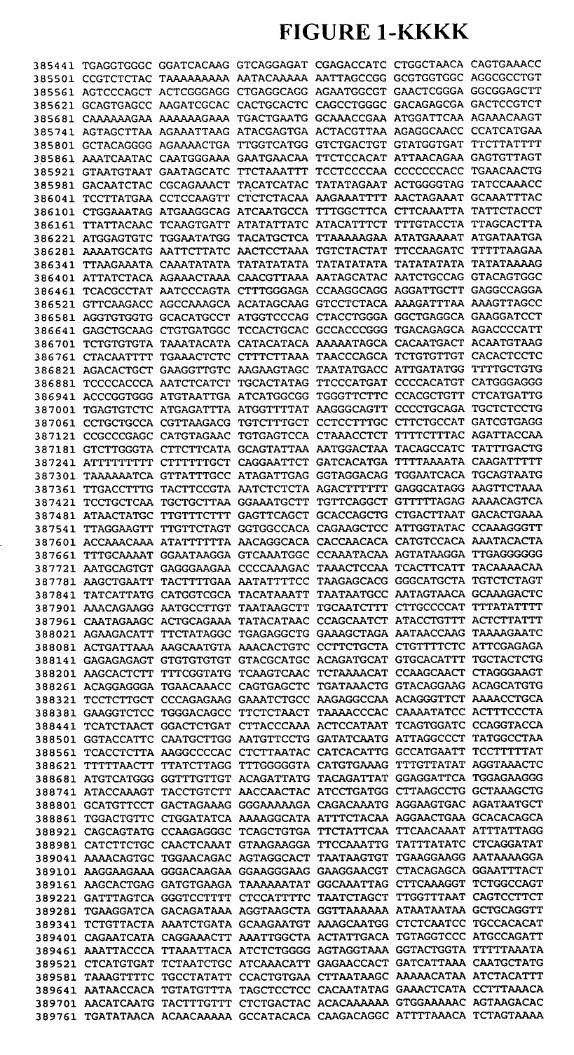

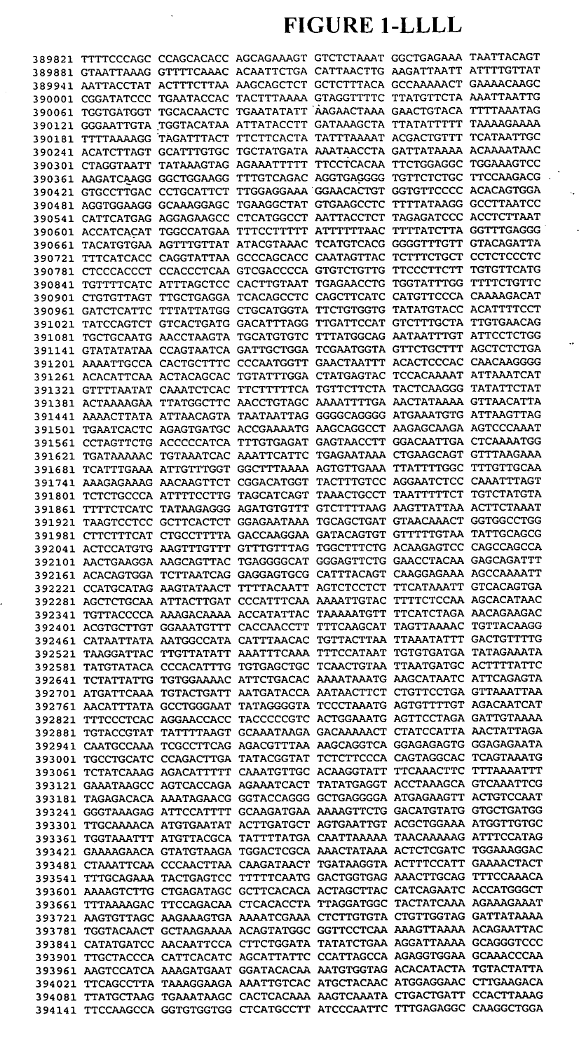

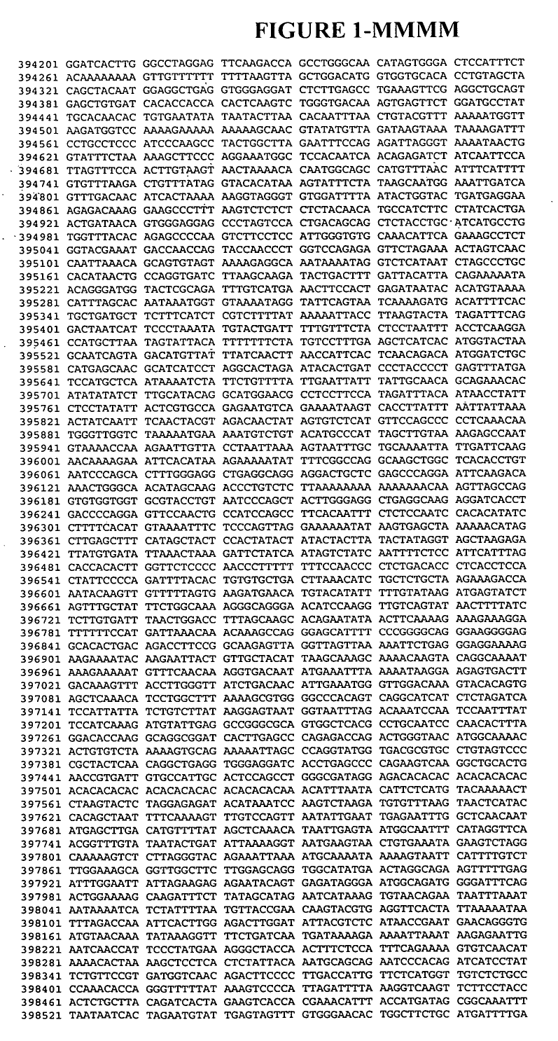

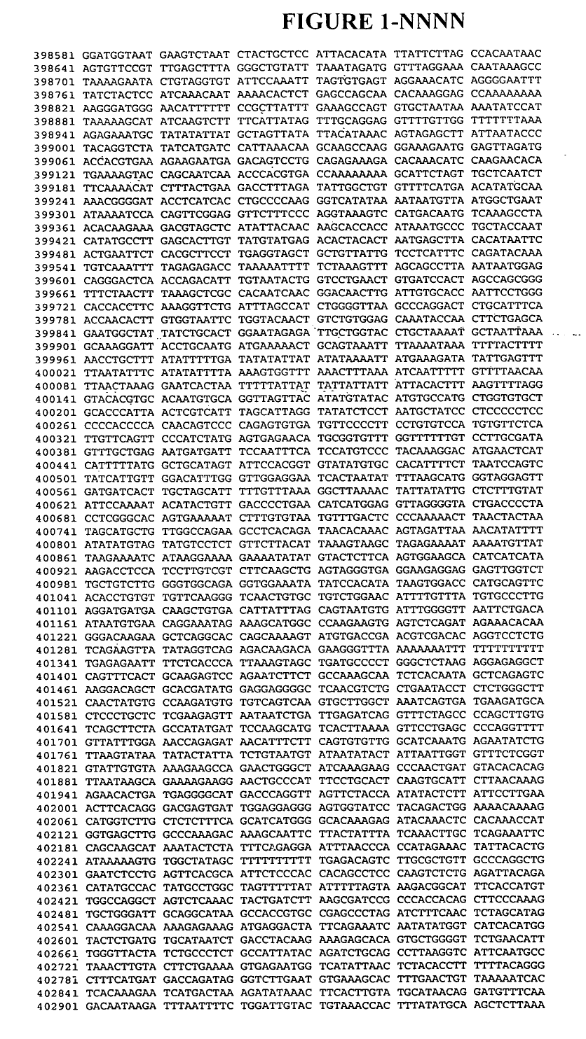

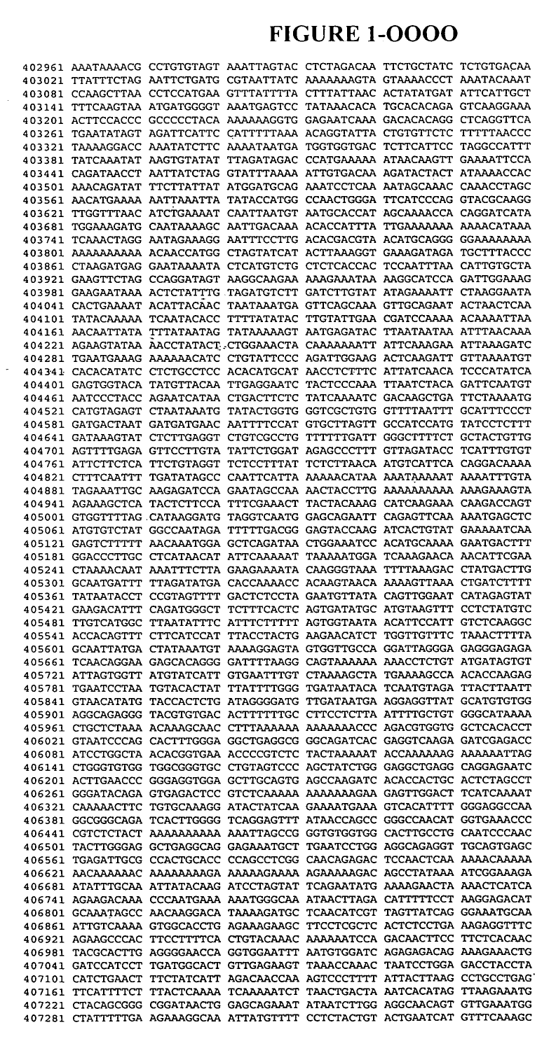

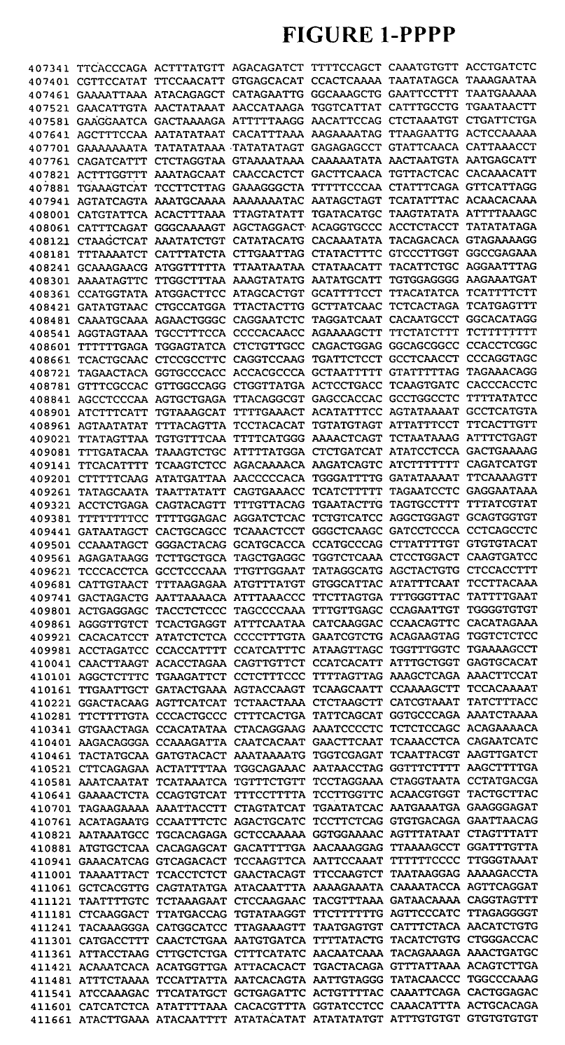

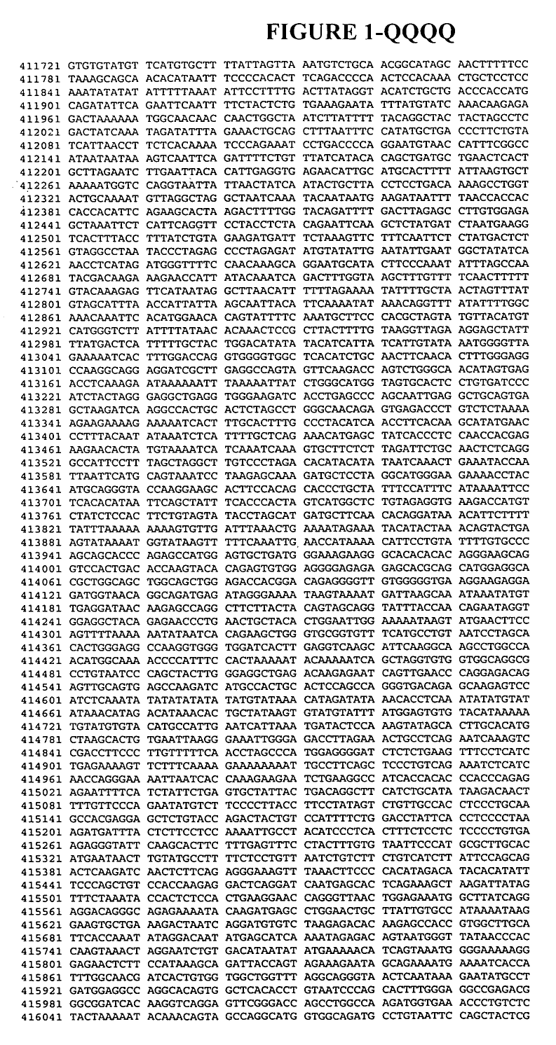

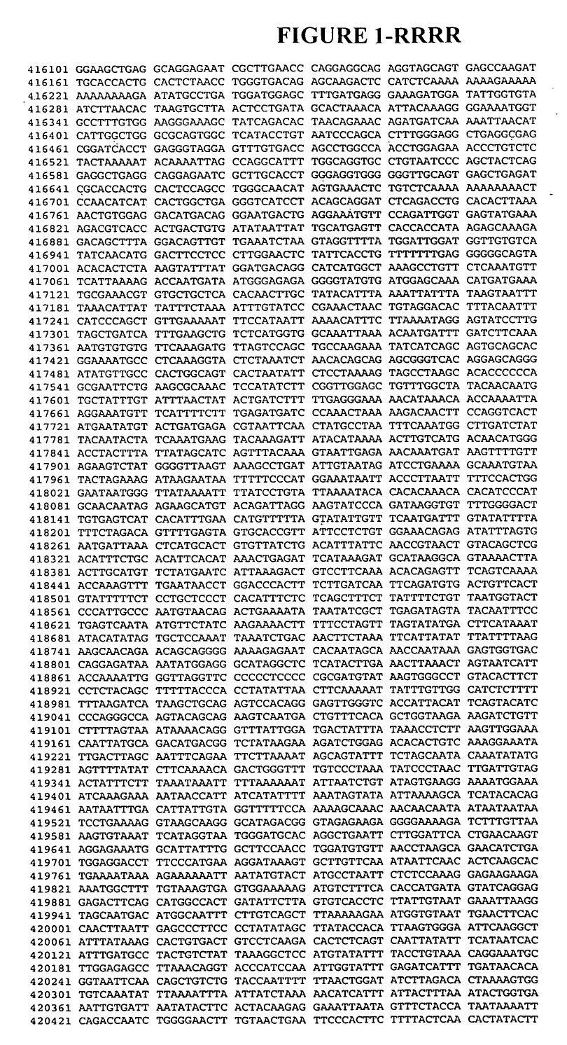

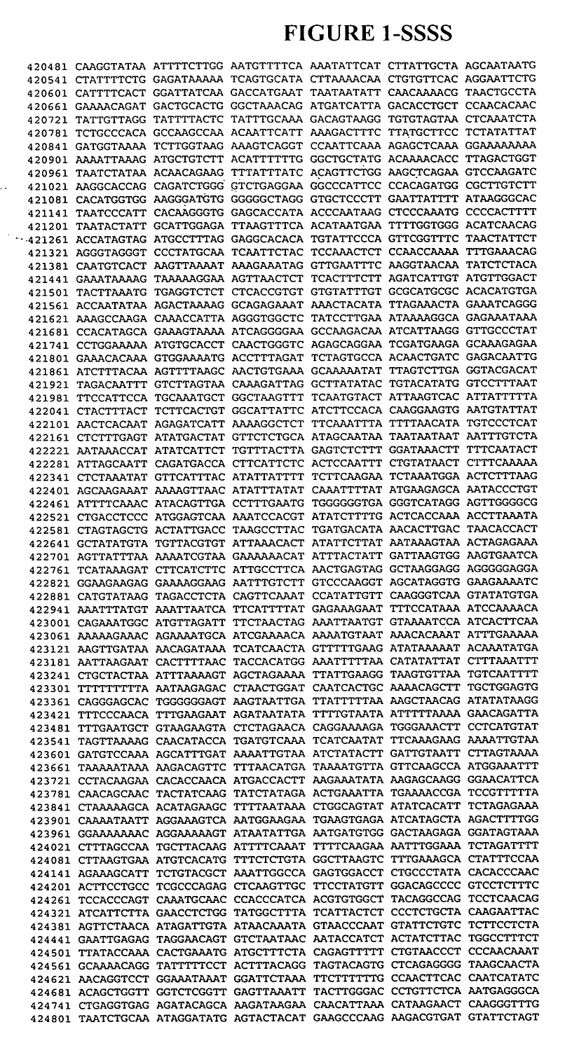

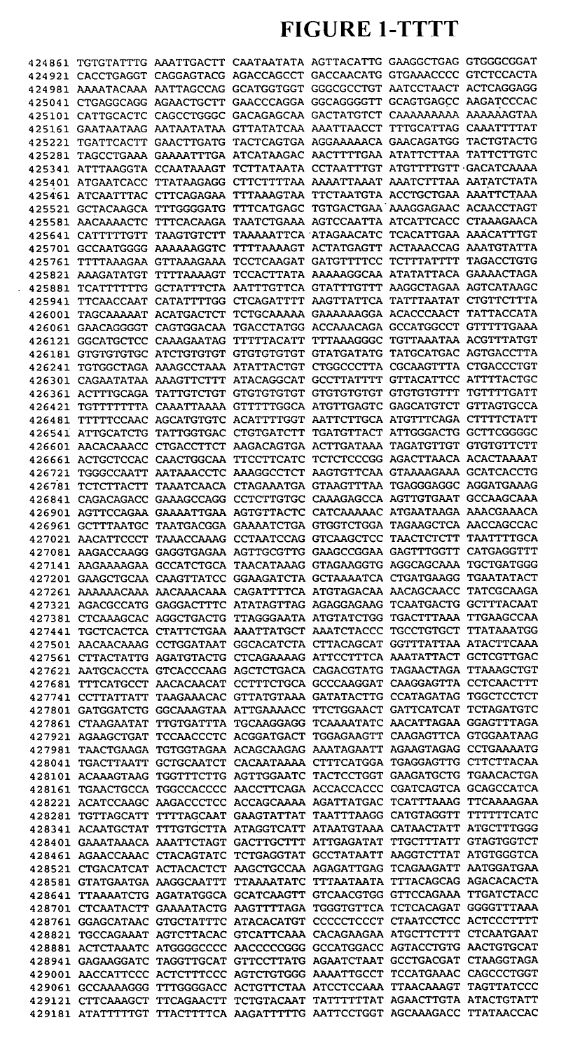

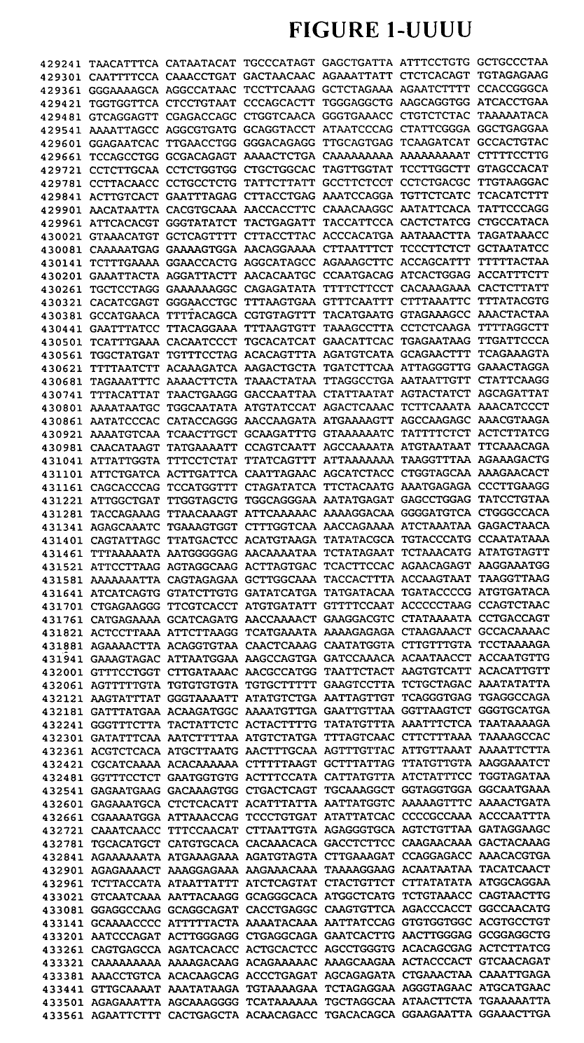

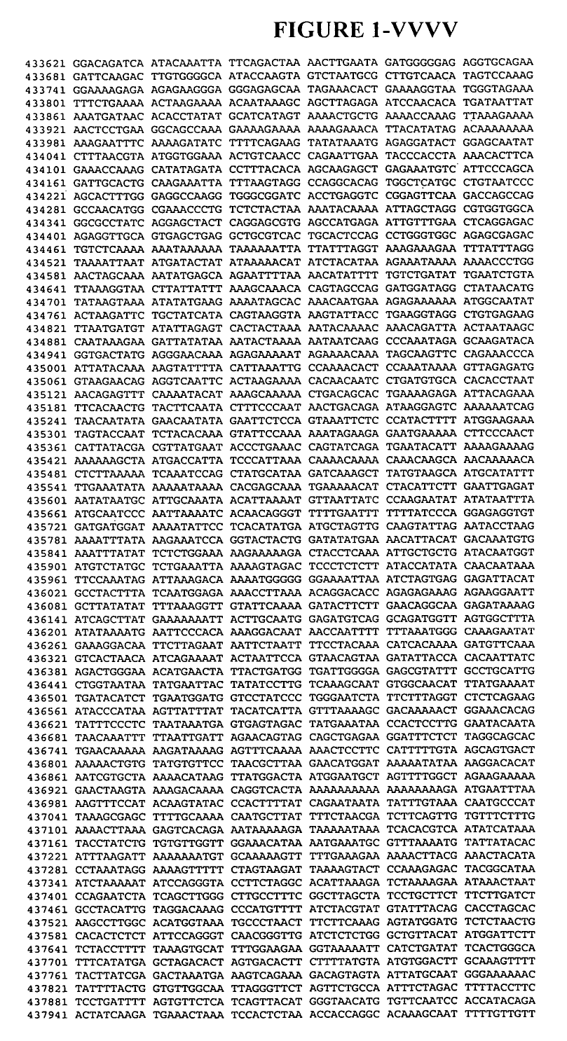

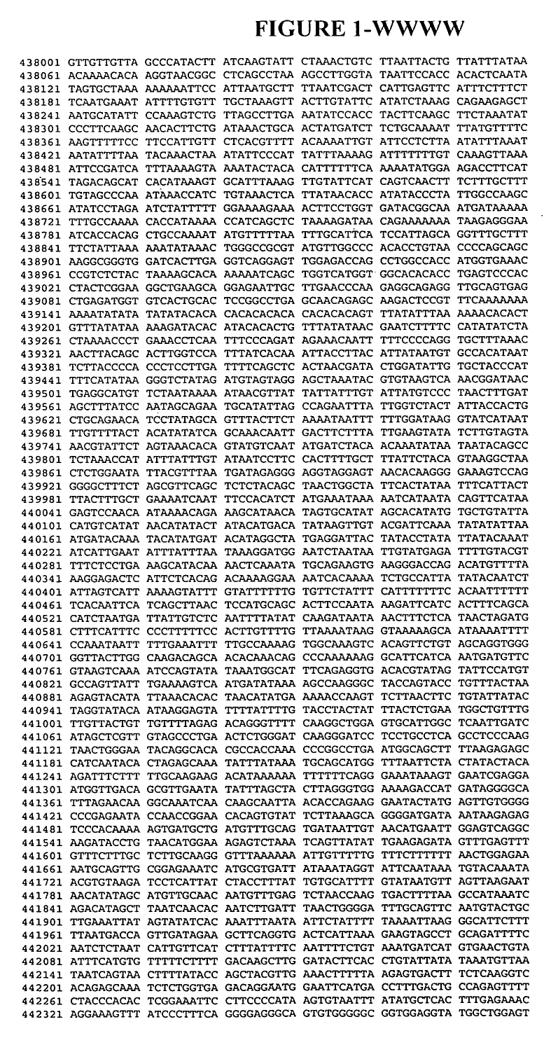

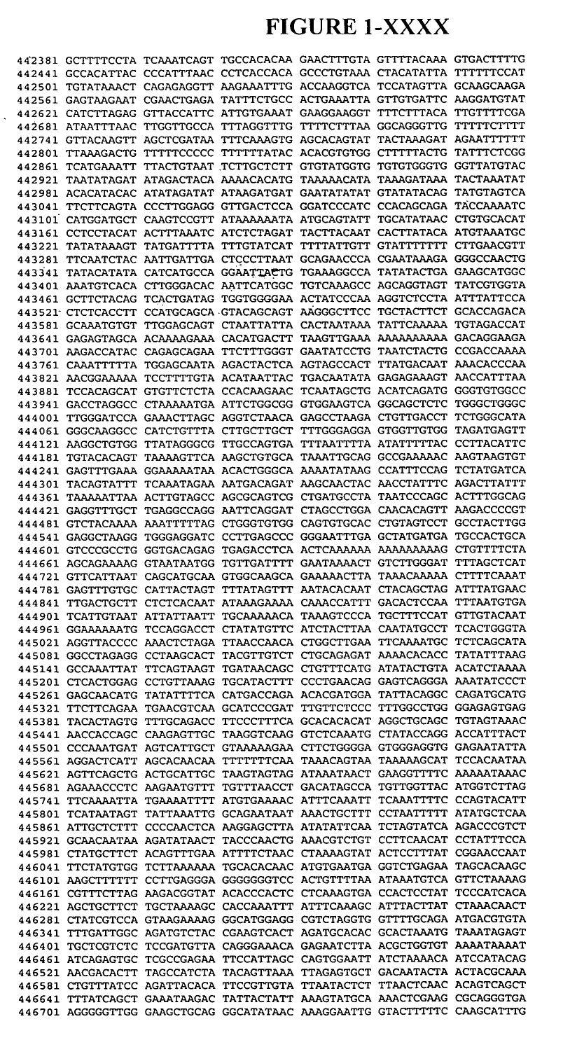

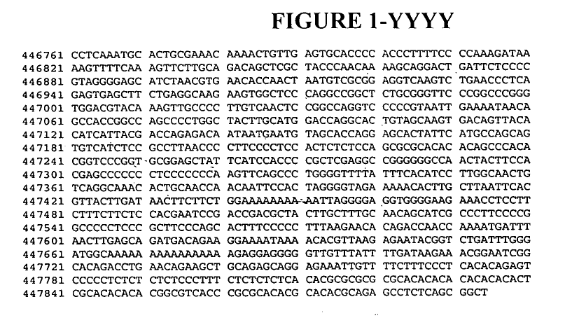

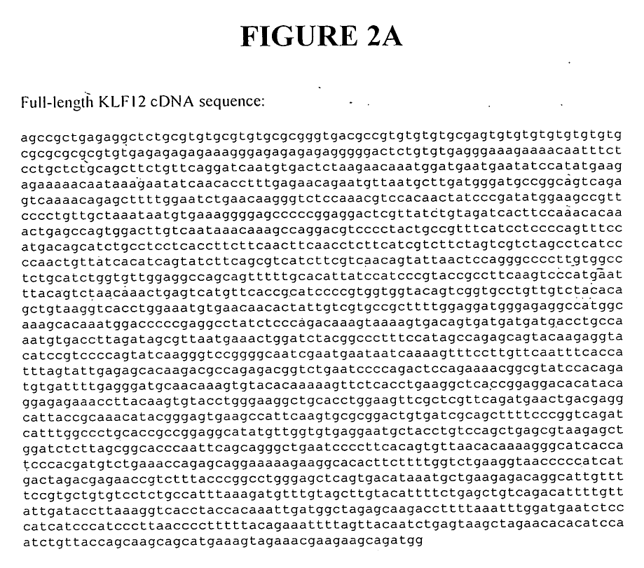

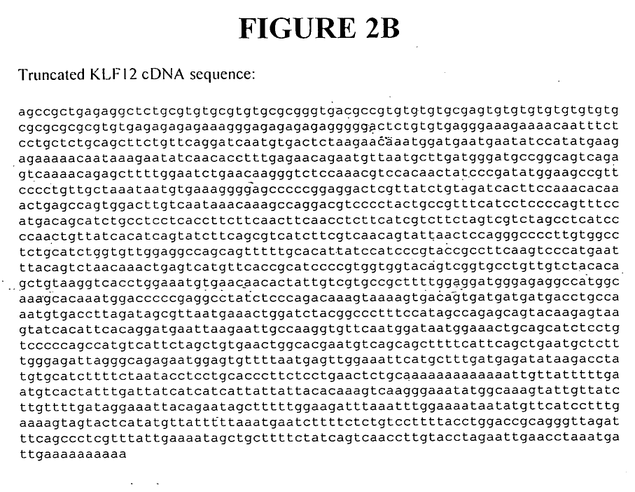

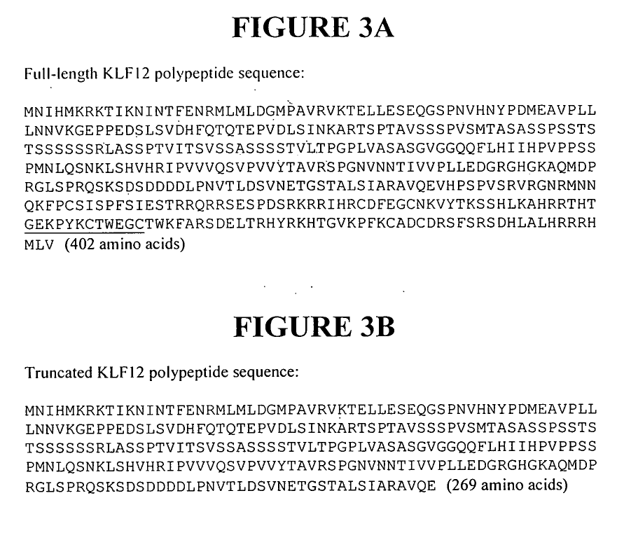

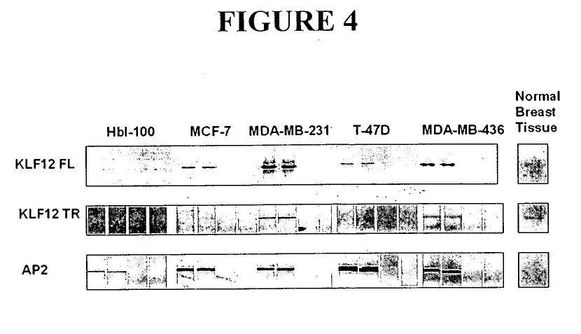

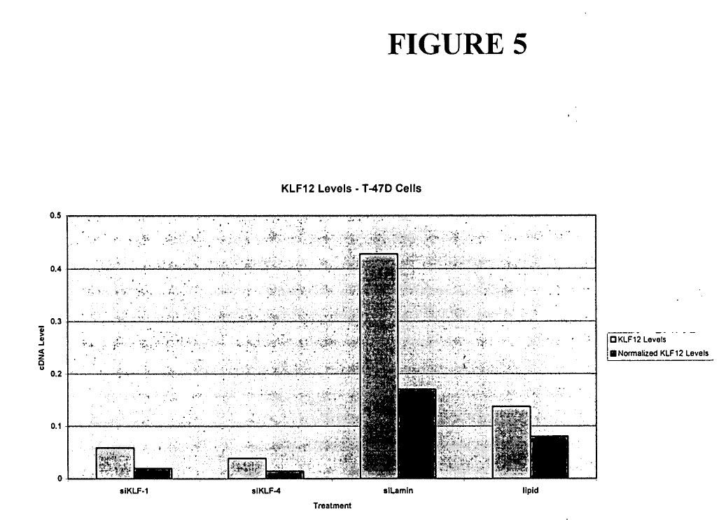

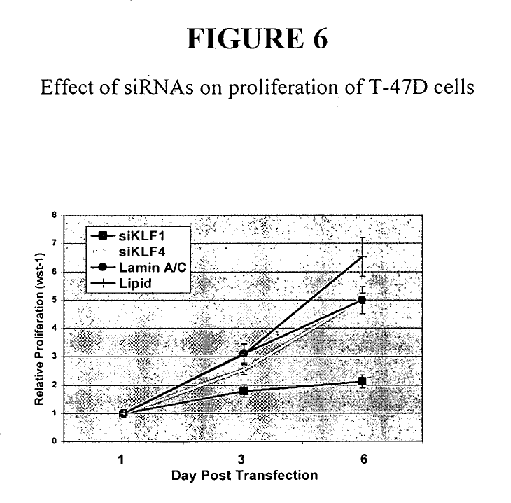

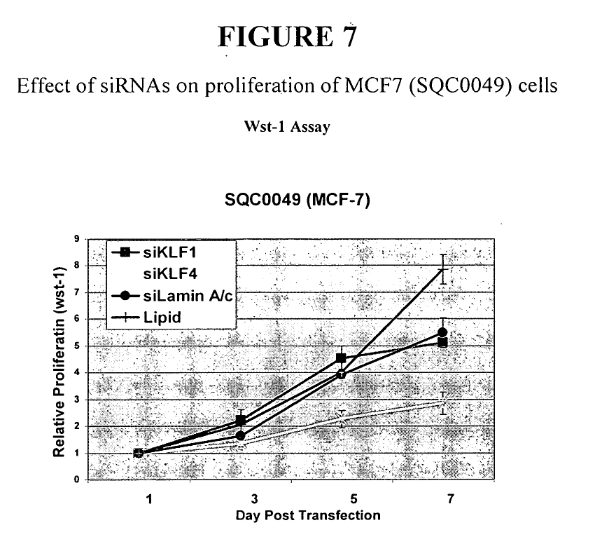

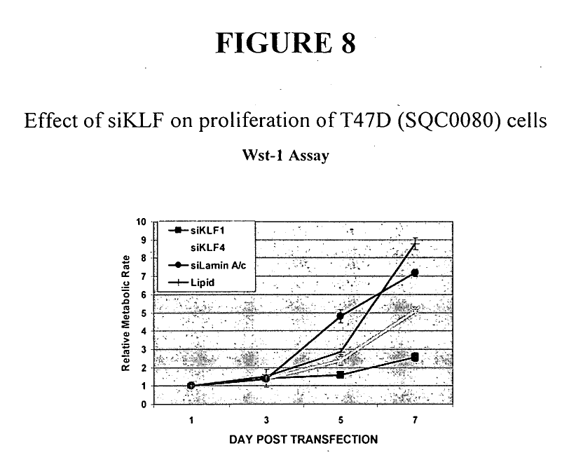

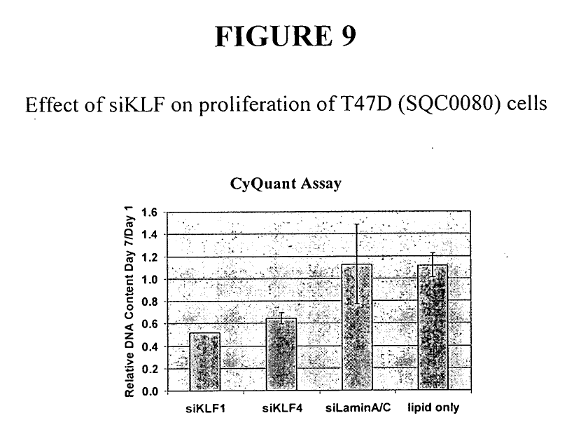

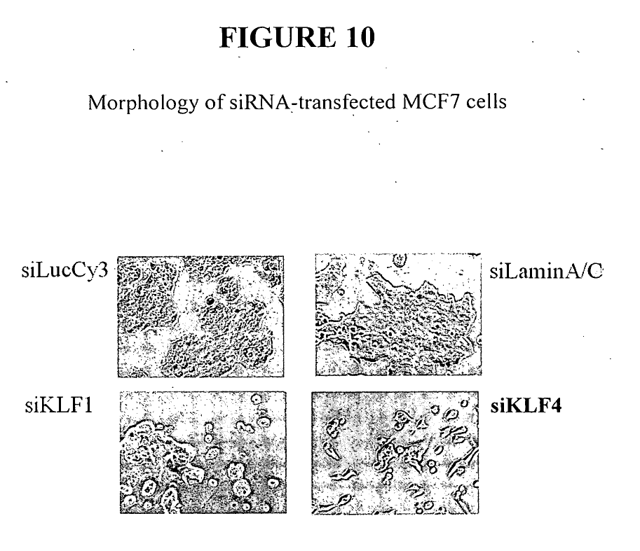

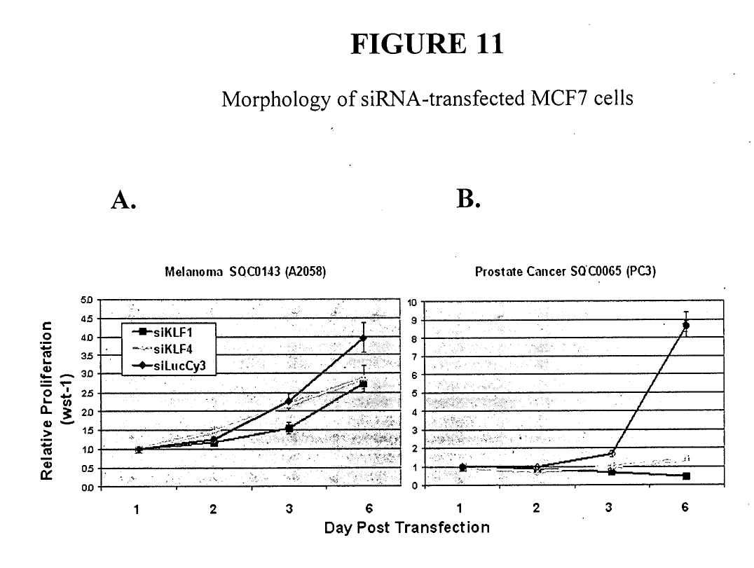

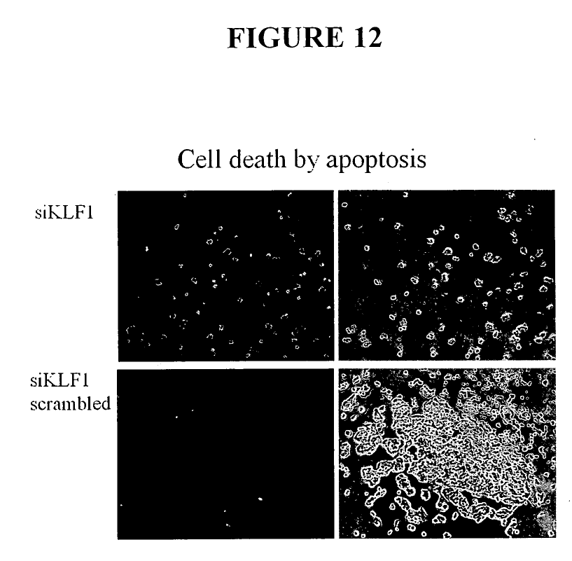

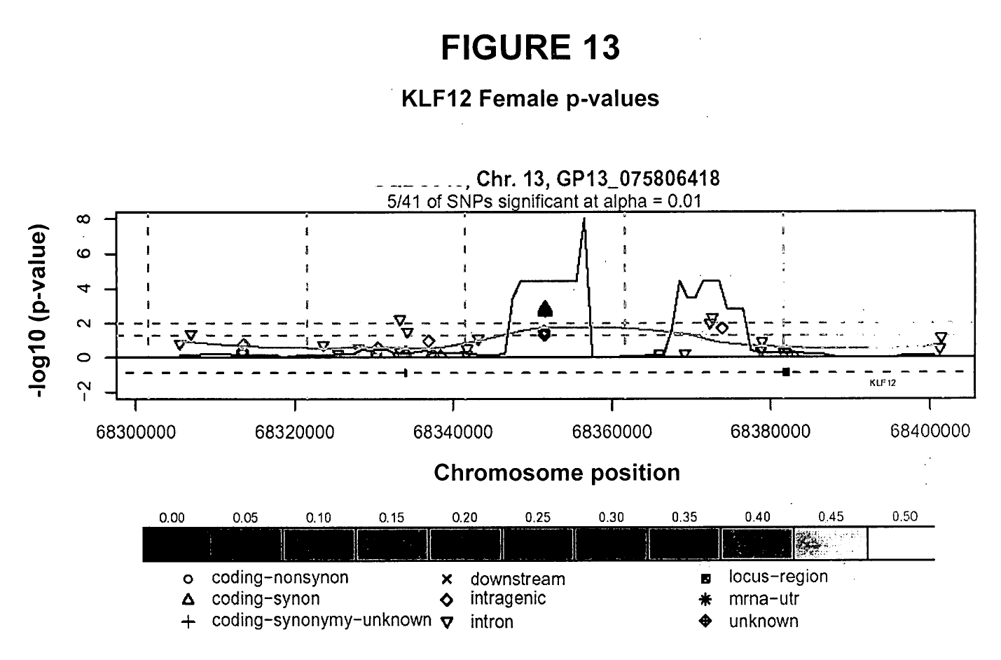

Also provided is a method for identifying polymorphic variants proximal to an incident, founder polymorphic variant. Thus, featured herein are methods for identifying one or more additional polymorphic variants associated with breast cancer, which comprise (a) providing a first polymorphic variant associated with breast cancer; (b) discovering or identifying (e.g. discovering a SNP that is not known or identifying a SNP that is known) at least a second polymorphic variant within a region in a nucleic acid including the first polymorphic variant, where the nucleic acid comprises: (i) a polynucleotide sequence set forth in FIG. 1 or FIGS. 2A-2B; (ii) a polynucleotide sequence that is 90% identical to a nucleotide sequence set forth in FIGS. 1A-1YYYY or FIGS. 2A-2B; (iii) a polynucleotide sequence that encodes a polypeptide having an amino acid sequence that is 90% identical to an amino acid sequence encoded by a nucleotide sequence set forth in FIGS. 1A-1YYYY or FIGS. 2A-2B; or (iv) a fragment of a polynucleotide sequence of (i), (ii), or (iii) comprising the polymorphic site; and (c) determining if the second polymorphic variant or other polymorphic variants is associated with cancer. In the above-described method, the identified second polymorphic variant sometimes is a known polymorphic variant as identified in a publicly available database. In certain embodiments, the first polymorphic variant is a polymorphic variation at position 96535 of SEQ ID NO: 1. In another embodiment, the first polymorphic variant is a polymorphic variation identified in Table 10 or Table 12. The region may be of any length, and in certain embodiments, the region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the first polymorphic variant. In certain embodiments, polymorphic variants associated with cancer are identified iteratively. For example, a third polymorphic variant can be identified or discovered in a region including the second polymorphic variant, and it can be determined whether the third polymorphic variant is associated with cancer. In another example, a fourth polymorphic variant can be identified or discovered in a region comprising the third polymorphic variant and it can be determined whether the third polymorphic variant is associated with cancer. In an embodiment, methods for determining if the polymorphic variant or other polymorphic variants are associated with cancer are directed to specific types of cancer, which include, but are not limited to, breast cancer, prostate cancer or melanoma.

The methods described herein are useful for identifying or discovering additional polymorphic variants that can be used to further characterize a gene, region or loci that are associated with a condition, a disease (e.g., cancer), or a disorder. For example, allelotyping or genotyping data from the additional polymorphic variants sometimes are used to identify a functional mutation or a region of linkage disequilibrium.

In certain embodiments, polymorphic variants identified or discovered within a region comprising the first polymorphic variant associated with cancer can be genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with cancer, and such information can be used in prognosis methods described herein.

Isolated KLF12 Nucleic Acids and Variants Thereof

Featured herein are isolated KLF12 nucleic acids, which include the genomic nucleic acids having the nucleotide sequence of SEQ ID NO: 1, the coding nucleic acids having the nucleotide sequence of SEQ ID NO: 2 which codes for full-length KLF12, the coding nucleic acid having the nucleotide sequence of SEQ ID NO: 3 which codes for truncated KLF12, KLF12 nucleic acid variants, and substantially identical nucleic acids to the foregoing. Nucleotide sequences of the KLF12 nucleic acids are sometimes referred to herein as “KLF12 nucleotide sequences.” A “KLF12 nucleic acid variant” refers to one allele that may have different polymorphic variations as compared to another allele in another subject or the same subject. A polymorphic variation in the KLF12 nucleic acid variant can be represented on one or both strands in a double-stranded nucleic acid or on one chromosomal complement (heterozygous) or both chromosomal complements (homozygous). A KLF12 nucleic acid may comprise any of the polymorphic variations described in Table 10 or Table 12. In addition, a nucleic acid complementary to the nucleic acids described in Table 10 or Table 12 may comprise the complementary base at the same polymorphic position, e.g., a cytosine at position 96535 of SEQ ID NO: 1 or a guanine at the same position in the complementary strand of nucleic acid.

As used herein, the term “nucleic acid” includes DNA molecules (e.g., a complementary DNA (cDNA) and genomic DNA (gDNA)) and RNA molecules (e.g., mRNA, rRNA, tRNA and siRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “KLF12 gene” refers to a nucleotide sequence that encodes a KLF12 polypeptide.

Also included herein are nucleic acid fragments. These fragments are often a nucleotide sequence identical to a nucleotide sequence in SEQ ID NOs: 1, 2 or 3, a nucleotide sequence substantially identical to a nucleotide sequence in SEQ ID NOs: 1, 2 or 3, or a nucleotide sequence that is complementary to the foregoing. The nucleic acid fragment is identical, substantially identical or homologous to a nucleotide sequence in an exon or an intron in SEQ ID NO: 1, or to a nucleotide sequence in an exon in SEQ ID NO: 2 or 3, and may encode a domain or part of a domain or motif of a KLF12 polypeptide. Domains and motifs of a KLF12 polypeptide include, but are not limited to, a zinc finger, C2H2 type, which corresponds to amino acid positions 378-395 of SEQ ID NO: 4. Sometimes, the fragment will comprises the polymorphic variation described herein as being associated with breast cancer. The nucleic acid fragment is often 50, 100, or 200 or fewer base pairs in length, and is sometimes about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3800, 4000, 5000, 6000, 7000, 8000, 9000, 10000, 15000, 20000, 30000, 40000, 50000, 60000, 70000, 80000, 90000, 100000, 110000, 120000, 130000, 140000, 150000, 200000, 250000, 300000, 350000, 400000 or 4500000 base pairs in length. A nucleic acid fragment that is complementary to a nucleotide sequence identical or substantially identical to the nucleotide sequence of SEQ ID NOs: 1, 2 or 3 and hybridizes to such a nucleotide sequence under stringent conditions is often referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein sometimes are used as hybridization probes or as components of prognostic or diagnostic assays, for example, as described herein.

Oligonucleotides are often synthesized using standard methods and equipment, such as the ABI™3900 High Throughput DNA Synthesizer and the EXPEDITE™8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614;622; 5,739,314; 5,955,599; 5,962,674; 6,117,992; in WO 00/75372; and in related publications.

Oligonucleotides may also be linked to a second moiety. The second moiety sometimes is an additional nucleotide sequence such as a tail sequence (e:g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses for Nucleic Acid Sequence

Nucleic acid coding sequences depicted in FIG. 1 or FIGS. 2A or 2B may be used for diagnostic purposes for detection and control of polypeptide expression. Also, included herein are oligonucleotide sequences such as antisense RNA, small-interfering RNA (siRNA), DNA molecules and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Ribozymes may be engineered hammerhead motif ribozyme molecules that specifically and efficiently catalyze endonucleolytic cleavage of RNA sequences corresponding to or complementary to the nucleotide sequences set forth in FIGS. 1A-1YYYY or FIGS. 2A-2B. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including cancer, often breast cancer, resulting from aberrant expression of the enzyme. For example, the nucleic acid sequence may be used in hybridization assays of biopsies or autopsies to diagnose abnormalities of expression or function (e.g., Southern or Northern blot analysis, in situ hybridization assays).

Expression Vectors, Host Cells, and Genetically Engineered Cells

Provided herein are nucleic acid vectors, often expression vectors, which contain a KLF12 nucleic acid. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a KLF12 nucleic acid in a form suitable for expression of the nucleic acid in a host cell. The recombinant expression vector often includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce KLF12 polypeptides, including fusion polypeptides, encoded by KLF12 nucleic acids.

Recombinant expression vectors can be designed for expression of KLF12 polypeptides in prokaryotic or eukaryotic cells. For example, KLF12 polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors often serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 3140 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for KLF12 polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol 43: 235-275 (1988)), promoters of T cell receptors (Winoto & Baltimore, EMBO J 8: 729-733 (1989)) promoters of immunoglobulins (Banerji et al., Cell 33: 729-740 (1983); Queen & Baltimore, Cell 33: 741-748 (1983)), neuron-specific promoters (e.g., the neurofilament promoter; Byme & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters are sometimes utilized, for example, the murine hox promoters (Kessel & Gruss, Science 249: 374-379 (1990)) and the alpha-fetopolypeptide promoter (Campes & Tilghman, Genes Dev. 3: 537-546 (1989)).

A KLF12 nucleic acid may also be cloned into an expression vector in an antisense orientation. Regulatory sequences (e.g., viral promoters and/or enhancers) operatively linked to a KLF12 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see Weintraub et al., Antisense RNA as a molecular tool for genetic analysis, Reviews—Trends in Genetics, Vol. 1(1) (1986).

Also provided herein are host cells that include a KLF12 nucleic acid within a recombinant expression vector or KLF12 nucleic acid sequence fragments which allow it to homologously recombine into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a KLF12 polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a KLF12 polypeptide. Accordingly, further provided are methods for producing a KLF12 polypeptide using the host cells described herein. In one embodiment, the method includes culturing host cells into which a recombinant expression vector encoding a KLF12 polypeptide has been introduced in a suitable medium such that a KLF12 polypeptide is produced. In another embodiment, the method further includes isolating a KLF12 polypeptide from the medium or the host cell.

Also provided are cells or purified preparations of cells which include a KLF12 transgene, or which otherwise misexpress KLF12 polypeptide. Cell preparations can consist of human or non-human cells, e.g., rodent cells, e.g., mouse or rat cells, rabbit cells, or pig cells. In certain embodiments, the cell or cells include a KLF12 transgene (e.g., a heterologous form of a KLF12 such as a human gene expressed in non-human cells). The KLF]2 transgene can be misexpressed, e.g., overexpressed or underexpressed. In other embodiments, the cell or cells include a gene which misexpress an endogenous KLF12 polypeptide (e.g., expression of a gene is disrupted, also known as a knockout). Such cells can serve as a model for studying disorders which are related to mutated or mis-expressed KLF12 alleles or for use in drug screening. Also provided are human cells (e.g., a hematopoietic stem cells) transformed with a KLF12 nucleic acid.

Also provided are cells or a purified preparation thereof (e.g., human cells) in which an endogenous KLF12 nucleic acid is under the control of a regulatory sequence that does not normally control the expression of the endogenous KLF12 gene. The expression characteristics of an endogenous gene within a cell (e.g., a cell line or microorganism) can be modified by inserting a heterologous DNA regulatory element into the genome of the cell such that the inserted regulatory element is operably linked to the endogenous KLF12 gene. For example, an endogenous KLF12 gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous KLF12 polypeptide (e.g., expressed from a KLF12 nucleic acid isolated from another organism) can be generated. Such animals are useful for studying the function and/or activity of a KLF12 polypeptide and for identifying and/or evaluating modulators of KLF12 nucleic acid and KLF12 polypeptide activity. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a KLF12 transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous KLF12 gene has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a KLF12 transgene to direct expression of a KLF12 polypeptide to particular cells. A. transgenic founder animal can be identified based upon the presence of a KLF12 transgene in its genome and/or expression of KLF12 mRNA in tissues or cells of the animals. A transgenic founder animal can then be used to breed additional animals carrying the transgene. Moreover, transgenic animals carrying a transgene encoding a KLF12 polypeptide can further be bred to other transgenic animals carrying other transgenes.

KLF12 polypeptides can be expressed in transgenic animals or plants by introducing, for example, a nucleic acid encoding the polypeptide into the genome of an animal. In certain embodiments the nucleic acid is placed under the control of a tissue specific promoter, e.g., a milk or egg specific promoter, and recovered from the milk or eggs produced by the animal. Also included is a population of cells from a transgenic animal.

KLF12 Polypeptides

Featured herein are isolated KLF12 polypeptides, which include polypeptides having amino acid sequences set forth in FIGS. 3A or 3B (SEQ ID NO: 4 or 5, respectively), and substantially identical polypeptides thereof. A KLF12 polypeptide is a polypeptide, protein or peptide encoded by a KLF12 nucleic acid, where one nucleic acid can encode one or more different polypeptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a KLF12 polypeptide or KLF12 polypeptide variant having less than about 30%, 20%, 10% and sometimes 5% (by dry weight), of non-KLF12 polypeptide (also referred to herein as a “contaminating protein”), or of chemical precursors or non-KLFI2 chemicals. When the KLF12 polypeptide or a biologically active portion thereof is recombinantly produced, it is also often substantially free of culture medium, specifically, where culture medium represents less than about 20%, sometimes less than about 10%, and often less than about 5% of the volume of the polypeptide. preparation. Isolated or purified KLF12 polypeptide preparations are sometimes 0.01 milligrams or more or 0.1 milligrams or more, and often 1.0 milligrams or more and 10 milligrams or more in dry weight.

In another aspect, featured herein are KLF12 polypeptides and biologically active or antigenic fragments thereof that are useful as reagents or targets in assays applicable to prevention, treatment or diagnosis of cancer. In another embodiment, provided herein are KLF12 polypeptides having a KLF12 activity or activities (e.g., AP-2a binding activity, WT1 mimicking activity, or gene silencing activity). In certain embodiments, the polypeptides are KLF12 proteins including at least one Gli-Kruppel C₂H₂-type zinc finger and sometimes having a KLF12 activity as described herein. Human KLF12 contains the following regions or other structural features: three CH2 type zinc finger domains at about amino acids 317-341, 347-371 and 377-399 of SEQ ID NO: 4.

In other embodiments, there are provided methods of decreasing the expression of KLF12, comprising providing or administering to individuals in need of decreasing the expression of KLF12 the pharmaceutical or physiologically acceptable composition comprising a KLF12 inhibitor (e.g., siRNA as described in Example 6).

Further included herein are KLF12 polypeptide fragments. The polypeptide fragment may be a domain or part of a domain of a KLF12 polypeptide. The polypeptide fragment is often 50 or fewer, 100 or fewer, or 200 or fewer amino acids in length, and is sometimes 300 or 400 or fewer amino acids in length. In certain embodiments, the polypeptide fragment comprises, consists essentially of, or consists of, at least 6 consecutive amino acids and not more than 402 consecutive amino acids of SEQ ID NO: 4, or the polypeptide fragment comprises, consists essentially of, or consists of, at least 6 consecutive amino acids and not more than 269 consecutive amino acids of SEQ ID NO: 5.

KLF12 polypeptides described herein can be used as immunogens to produce anti-KLF12 antibodies in a subject, to purify KLF12 ligands or binding partners, and in screening assays to identify molecules which inhibit or enhance the interaction of KLF12 with a KLF12 substrate. In an embodiment, KLF12 polypeptides described herein are used to screen for inhibitors of KLF12 expression. Full-length KLF12 polypeptides and polynucleotides encoding the same may be specifically substituted for a KLF12 polypeptide fragment or polynucleotide encoding the same in any embodiment described herein.

Substantially identical polypeptides may depart from the amino acid sequences set forth in FIGS. 3A or 3B in different manners. For example, conservative amino acid modifications may be introduced at one or more positions in the amino acid sequences of FIGS. 3A or 3B. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a KLF12 polypeptide, whereas altering an “essential” amino acid abolishes or substantially alters the biological function of a KLF12 polypeptide. Amino acids that are conserved among KLF12 polypeptides are often essential amino acids.

Also, KLF12 polypeptides and polypeptide variants may exist as chimeric or fusion polypeptides. As used herein, a KLF12 “chimeric polypeptide” or “fusion polypeptide” includes a KLF12 polypeptide linked to a non-KLF12 polypeptide. A “non-KLF12 polypeptide” refers to a polypeptide having an amino acid sequence corresponding to a polypeptide which is not substantially identical to the KLF12 polypeptide, which includes, for example, a polypeptide that is different from the KLF12 polypeptide and derived from the same or a different organism. The KLF12 polypeptide in the fusion polypeptide can correspond to an entire or nearly entire KLF12 polypeptide or a fragment thereof. The non-KLF12 polypeptide can be fused to the N-terminus or C-terminus of the KLF12 polypeptide.

Fusion polypeptides can include a moiety having high affinity for a ligand. For example, the fusion polypeptide can be a GST-KLF12 fusion polypeptide in which the KLF12 sequences are fused to the C-terminus of the GST sequences, or a polyhistidine-KLF12 fusion polypeptide in which the KLF12 polypeptide is fused at the N- or C-terminus to a string of histidine residues. Such fusion polypeptides can facilitate purification of recombinant KLF12. Expression vectors are commercially available that already encode a fusion moiety (e.g., a GST polypeptide), and a KLF12 nucleic acid can be cloned into an expression vector such that the fusion moiety is linked in-frame to the KLF12 polypeptide. Further, the fusion polypeptide can be a KLF12 polypeptide containing a heterologous signal sequence at its N-terminus. In certain host cells (e.g., mammalian host cells), expression, secretion, cellular internalization, and cellular localization of a KLF12 polypeptide can be increased through use of a heterologous signal sequence. Fusion polypeptides can also include all or a part of a serum polypeptide (e.g., an IgG constant region or human serum albumin).

KLF12 polypeptides or fragments thereof can be incorporated into pharmaceutical compositions and administered to a subject in vivo. Administration of these KLF12 polypeptides can be used to affect the bioavailability of a KLF12 substrate and may effectively increase or decrease KLF12 biological activity in a cell or effectively supplement dysfunctional or hyperactive KLF12 polypeptide. KLF12 fusion polypeptides may be useful therapeutically for the treatment of disorders caused by, for example, (i) aberrant modification or mutation of a gene encoding a KLF12 polypeptide; (ii) mis-regulation of the KLF12 gene; and (iii) aberrant post-translational modification of a KLF12 polypeptide. Also, KLF12 polypeptides can be used as immunogens to produce anti-KLF12 antibodies in a subject, to purify KLF12 ligands or binding partners, and in screening assays to identify molecules which inhibit or enhance the interaction of KLF12 with a KLF12 substrate. The KLF12 polypeptides often are used in screening assays to identify molecules which inhibit the over-expression of KLF12.

In addition, polypeptides can be chemically synthesized using techniques known in the art (See, e.g., Creighton, 1983 Proteins. New York, N.Y.: W. H. Freeman and Company; and Hunkapiller et al., (1984) Nature July 12-18;310(5973):105-11). For example, a relative short polypeptide fragment can be synthesized by use of a peptide synthesizer. Furthermore, if desired, non-classical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Also included are polypeptide fragments which are differentially modified during or after translation, e.g., by glycosylation, acetylation, phosphorylation, amidation, derivatization by known protecting/blocking groups, proteolytic cleavage, linkage to an antibody molecule or other cellular ligand, and the like. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptide fragments may also be modified with a detectable label, such as an enzymatic, fluorescent, isotopic or affinity label to allow for detection and isolation of the polypeptide.

Also provided are chemically modified polypeptide derivatives that may provide additional advantages such as increased solubility, stability and circulating time of the polypeptide, or decreased immunogenicity. See U.S. Pat. No: 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the polypeptide with consideration of effects on functional or antigenic domains of the polypeptide. There are a number of attachment methods available to those skilled in the art, e.g., EP 0 401 384, herein incorporated by reference (coupling PEG to G-CSF), see also Malik et al. (1992) Exp Hematol. September;20(8):1028-35, reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. A polymer sometimes is attached at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, and the like), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical KLF12 Nucleic Acids and Polypeptides

KLF12 nucleotide sequences and KLF12 polypeptide sequences that are substantially identical to the nucleotide sequences of FIGS. 1, 2A or 2B and the polypeptide sequences of FIGS. 3A or 3B, respectively, are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55% or more, 60% or more, 65% or more, 70% or more, 75% or more, 80% or more, 85% or more, 90% or more, 95% or more (each often within a 1%, 2%, 3% or 4% variability) or more identical to the KLF12 nucleotide sequences in FIGS. 1A-1YYYY (SEQ ID NO: 1), FIG. 2A or 2B (SEQ ID NO: 2 or 3), or the KLF12 polypeptide sequences of FIG. 3A or 3B (SEQ ID NO: 4 or 5). One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the sane nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444453 (1970) algorithm which has been incorporated into the GAP program in the GCG software package (available at the http address www.gcg.com), using either a Blossum 62 matrix or a PAM250 matrix, and a gap-weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1 989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to SEQ ID NO: 1 is one that has a different nucleotide sequence and still encodes a polypeptide sequence set forth in FIG. 3A or 3B. Another example is a nucleotide sequence that encodes a polypeptide having a polypeptide sequence that is 70% or more identical to, sometimes 75% or more, 80% or more, or 85% or more identical to, and often 90% or more and 95% or more identical to the polypeptide sequences set forth in FIGS. 3A or 3B.

KLF12 nucleotide sequences and polypeptide sequences can be used as “query sequences” to perform a search against public databases to identify other family members or related sequences, for example. Such searches can be performed using the NBLAST and XBLAST programs (version 2.0) of Altschul et al, J. Mol. Biol. 215: 403-10 (1990). BLAST nucleotide searches can be performed with the NBLAST program, score=100, wordlength=12 to obtain nucleotide sequences homologous to KLF12 nucleic acid molecules. BLAST polypeptide searches can be performed with the XBLAST program, score=50, wordlength=3 to obtain amino acid sequences homologous to KLF12 polypeptides. To obtain gapped alignments for comparison purposes, Gapped BLAST can be utilized as described in Altschul et al, Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nim.nih.gov).

A nucleic acid that is substantially identical to the nucleotide sequences of SEQ ID NO: 1, 2 or 3 may include polymorphic sites at positions equivalent to those described herein (e.g., position 96535 in SEQ ID NO: 1) when the sequences are aligned. For example, using the alignment procedures described herein, SNPs in a sequence substantially identical to the sequences of SEQ ID NO: 1, 2 or 3 can be identified at nucleotide positions that match (i.e., align) with nucleotides at SNP positions in SEQ ID NO: 1, 2 or 3. Also, where a polymorphic variation is an insertion or deletion, insertion or deletion of a nucleotide sequence from a reference sequence can change the relative positions of other polymorphic sites in the nucleotide sequence.

Substantially identical KLF12 nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50% or more, about 55% or more, often about 70-75% or more, more often about 80-85% or more, and often about 90-95% or more identical to the amino acid sequences shown in FIGS. 3A or 3B or a fragment thereof. Such nucleic acid molecules can readily be identified as being able to hybridize under stringent conditions to the nucleotide sequences shown in SEQ ID NO: 1, 2 or 3 or a fragment of the sequence. Nucleic acid molecules corresponding to orthologs, homologs, and allelic variants of the KLF12 nucleotide sequence can further be identified by mapping the sequence to the same chromosome or locus as the KLF12 nucleotide sequence or variant.

Also, substantially identical KLF12 nucleotide sequences may include codons that are altered with respect to the naturally occurring sequence for enhancing expression of a KLF12 polypeptide or polypeptide variant in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect, or rodent (e.g., hamster) cells.

Methods for Identifying Risk of Cancer and Subjects at Risk of Cancer

Methods for identifying risk of cancer are provided, which includes determining the risk of having or developing cancer and/or identifying subjects at risk of cancer, and also are referred to as prognostic or diagnostic methods. These methods include detecting the presence or absence of one or more polymorphic variations in a nucleotide sequence set forth in FIG. 1 or FIG. 2, or a substantially identical nucleotide sequence thereof, in a sample from a subject, where the presence of a polymorphic variant described herein is indicative of a risk of cancer. Identifying a predisposition to cancer refers to determining whether an individual is at risk of developing cancer. In addition, detecting the presence or absence of one or more polymorphic variations in a nucleotide sequence of FIG. 1 or FIG. 2, or substantially identical sequence thereof, in a sample from a subject maybe diagnostic of the presence of cancer in the individual. In certain embodiments, the cancer is breast cancer, prostate cancer or melanoma.

Methods for identifying risk of cancer may be applied to any type of cancer such as breast cancer. As described hereafter, results from prognostic tests may be combined with other test results to diagnose breast cancer. For example, prognostic results may be gathered, a mammogram, clinical breast exam (CBE), or biopsy may be ordered based on a determined predisposition to breast cancer, and the mammogram, clinical breast exam or biopsy results may be utilized to diagnose breast cancer. Also breast cancer diagnostic methods can be developed from studies used to generate prognostic methods in which breast cancer populations are stratified into subpopulations having different progressions of the cancer.

Thus, featured herein is a method for detecting a subject at risk of breast cancer or the risk of breast cancer in a subject, which comprises detecting the presence or absence of a polymorphic variation associated with breast cancer at a polymorphic site in a nucleotide sequence set forth in SEQ ID NO: 1, 2 or 3 in a nucleic acid sample from a subject, where the nucleotide sequence comprises a polynucleotide sequence selected from the group consisting of: (a) a nucleotide sequence set forth in SEQ ID NO: 1, 2 or 3; (b) a nucleotide sequence which encodes a polypeptide having an amino acid sequence encoded by a nucleotide sequence in SEQ ID NO: 1, 2 or 3; (c) a nucleotide sequence which encodes a polypeptide that is 90% or more identical to an amino acid sequence encoded by a nucleotide sequence in SEQ ID NO: 1, 2 or 3 or a nucleotide sequence about 90% or more identical to the nucleotide sequence set forth in SEQ ID NO: 1, 2 or 3; and (d) a fragment of a nucleotide sequence of (a), (b), or (c), often a fragment that includes a polymorphic site associated with breast cancer; whereby the presence of the polymorphic variation is indicative of a risk of breast cancer in the subject. In certain embodiments, determining the presence of a combination of two or more polymorphic variants associated with breast cancer in one or more nucleotide sequences of the sample (e.g., at ICAM, MAPK10, NUMA1, KLF12 and/or GALE sequences) is determined to identify a subject at risk of breast cancer and/or risk of breast cancer.

A risk of developing aggressive forms of breast cancer likely to metastasize or invade surrounding tissues (e.g., Stage IIIA, IIIB, and IV breast cancers), and subjects at risk of developing aggressive forms of breast cancer also may be identified by the methods described herein. These methods include collecting phenotype information from subjects having breast cancer, which includes the stage of progression of the breast cancer, and performing a secondary phenotype analysis to detect the presence or absence of one or more polymorphic variations associated with a particular stage form of breast cancer. Thus, detecting the presence or absence of one or more polymorphic variations in a KLF12 nucleotide sequence associated with a late stage form of breast cancer often is diagnostic of an aggressive form of the cancer.

Results from prognostic tests may be combined with other test results to diagnose breast cancer. For example, prognostic results may be gathered, a patient sample may be ordered based on a determined predisposition to breast cancer, the patient sample is analyzed, and the results of the analysis may be utilized to diagnose breast cancer. Also breast cancer diagnostic methods can be developed from studies used to generate prognostic/diagnostic methods in which populations are stratified into subpopulations having different progressions of breast cancer. In another embodiment, prognostic results may be gathered; a patient's risk factors for developing breast cancer analyzed (e.g., age, race, family history, age of first menstrual cycle, age at birth of first child); and a patient sample may be ordered based on a determined predisposition to breast cancer. In an alternative embodiment, the results from predisposition analyses described herein may be combined with other test results indicative of breast cancer, which were previously, concurrently, or subsequently gathered with respect to the predisposition testing. In these embodiments, the combination of the prognostic test results with other test results can be probative of breast cancer, and the combination can be utilized as a breast cancer diagnostic. The results of any test indicative of breast cancer known in the art may be combined with the methods described herein. Examples of such tests are mammography (e.g., a more frequent and/or earlier mammography regimen may be prescribed); breast biopsy and optionally a biopsy from another tissue; breast ultrasound and optionally an ultrasound analysis of another tissue; breast magnetic resonance imaging (MRI) and optionally an MRI analysis of another tissue; electrical impedance (T-scan) analysis of breast and optionally of another tissue; ductal lavage; nuclear medicine analysis (e.g., scintimammography); BRCA1 and/or BRCA2 sequence analysis results; and thermal imaging of the breast and optionally of another tissue. Testing may be performed on tissue other than breast to diagnose the occurrence of metastasis (e.g., testing of the lymph node).

Risk of breast cancer sometimes is expressed as a probability, such as an odds ratio, percentage, or risk factor. The risk is based upon the presence or absence of one or more polymorphic variants described herein, and also may be based in part upon phenotypic traits of the individual being tested. Methods for calculating risk based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations. Risk determinations for breast cancer are useful in a variety of applications. In one embodiment, breast cancer risk determinations are used by clinicians to direct appropriate detection, preventative and treatment procedures to subjects who most require these. In another embodiment, breast cancer risk determinations are used by health insurers for preparing actuarial tables and for calculating insurance premiums.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (eg., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202,4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon a nucleotide sequence set forth in SEQ ID NO: 1, 2 or 3 without undue experimentation using knowledge readily available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2. Multiple extension oligonucleotides may be utilized in one reaction, which is referred to herein as “multiplexing.”

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site set forth in FIG. 1 or below.

A kit also may be utilized for determining whether a polymorphic variant is present or absent in a nucleic acid sample. A kit often comprises one or more pairs of oligonucleotide primers useful for amplifying a fragment of a KLF12 nucleotide sequence or a substantially identical sequence thereof, where the fragment includes a polymorphic site. The kit sometimes comprises a polymerizing agent, for example, a thermostable nucleic acid polymerase such as one disclosed in U.S. Pat. Nos. 4,889,818 or 6,077,664. Also, the kit often comprises an elongation oligonucleotide that hybridizes to a KLF12 nucleotide sequence in a nucleic acid sample adjacent to the polymorphic site. Where the kit includes an elongation oligonucleotide, it also often comprises chain elongating nucleotides, such as dATP, dTTP, dGTP, dCTP, and dITP, including analogs of dATP, dTTP, dGTP, dCTP and dTTP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

An individual identified as being at risk of breast cancer may be heterozygous or homozygous with respect to the allele associated with a higher risk of breast cancer. A subject homozygous for an allele associated with an increased risk of breast cancer is at a comparatively high risk of breast cancer, a subject heterozygous for an allele associated with an increased risk of breast cancer is at a comparatively intermediate risk of breast cancer, and a subject homozygous for an allele associated with a decreased risk of breast cancer is at a comparatively low risk of breast cancer. A genotype may be assessed for a complementary strand, such that the complementary nucleotide at a particular position is detected.

Also featured are methods for determining risk of breast cancer and/or identifying a subject at risk of breast cancer by contacting a polypeptide or protein encoded by a KLF12 nucleotide sequence from a subject with an antibody that specifically binds to an epitope associated with increased risk of breast cancer in the polypeptide.

Applications of Prognostic and Diagnostic Results to Pharmacogenomic Methods

Pharmacogenomics is a discipline that involves tailoring a treatment for a subject according to the subject's genotype. For example, based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects). As therapeutic approaches for breast cancer continue to evolve and improve, the goal of treatments for breast cancer related disorders is to intervene even before clinical signs (e.g., identification of lump in the breast) first manifest. Thus, genetic markers associated with susceptibility to breast cancer prove useful for early diagnosis, prevention and treatment of breast cancer.

The following is an example of a pharmacogenomic embodiment. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homoygous for the minor allele, and sometimes not administered to a subject genotyped as being heteroygous for the minor allele. In another example, where a candidate therapeutic is not significantly toxic when administered to subjects who are homoygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for detecting, preventing, alleviating and/or treating breast cancer. For example, a nucleic acid sample from an individual may be subjected to a genetic test described herein. Where one or more polymorphic variations associated with increased risk of breast cancer are identified in a subject, information for detecting, preventing or treating breast cancer and/or one or more breast cancer detection, prevention and/or treatment regimens then may be directed to and/or prescribed to that subject.

In certain embodiments, a detection, prevenative and/or treatment regimen is specifically prescribed and/or administered to individuals who will most benefit from it based upon their risk of developing breast cancer assessed by the methods described herein. Thus, provided are methods for identifying a subject at risk of breast cancer and then prescribing a detection, therapeutic or preventative regimen to individuals identified as being at risk of breast cancer. Thus, certain embodiments are directed to methods for treating breast cancer in a subject, reducing risk of breast cancer in a subject, or early detection of breast cancer in a subject, which comprise: detecting the presence or absence of a polymorphic variant associated with breast cancer in a nucleotide sequence set forth in SEQ ID NO: 1, 2 or 3 in a nucleic acid sample from a subject, where the nucleotide sequence comprises a polynucleotide sequence selected from the group consisting of: (a) a nucleotide sequence set forth in SEQ ID NO: 1, 2 or 3; (b) a nucleotide sequence which encodes a polypeptide having an amino acid sequence encoded by a nucleotide sequence in SEQ ID NO: 1, 2 or 3; (c) a nucleotide sequence which encodes a polypeptide that is 90% or more identical to an amino acid sequence encoded by a nucleotide sequence in SEQ ID NO: 1, 2 or 3 or a nucleotide sequence about 90% or more identical to the nucleotide sequence set forth in SEQ ID NO: 1, 2 or 3; and (d) a fragment of a nucleotide sequence of (a), (b), or (c), sometimes comprising a polymorphic site associated with breast cancer; and prescribing or administering a breast cancer treatment regimen, preventative regimen and/or detection regimen to a subject from whom the sample originated where the presence of one or more polymorphic variations associated with breast cancer are detected in the nucleotide sequence. In these methods, genetic results may be utilized in combination with other test results to diagnose breast cancer as described above. Other test results include but are not limited to mammography results, imaging results, biopsy results and results from BRCA1 or BRAC2 test results, as described above.

Detection regimens include one or more mammography procedures, a regular mammography regimen (e.g., once a year, or once every six, four, three or two months); an early mammography regimen (e.g., mammography tests are performed beginning at age 25, 30, or 35); one or more biopsy procedures (e.g., a regular biopsy regimen beginning at age 40); breast biopsy and biopsy from other tissue; breast ultrasound and optionally ultrasound analysis of another tissue; breast magnetic resonance imaging (MRI) and optionally MRI analysis of another tissue; electrical impedance (T-scan) analysis of breast and optionally another tissue; ductal lavage; nuclear medicine analysis (e.g., scintimammography); BRCA1 and/or BRCA2 sequence analysis results; and/or thermal imaging of the breast and optionally another tissue.

Treatments sometimes are preventative (e.g., is prescribed or administered to reduce the probability that a breast cancer associated condition arises or progresses), sometimes are therapeutic, and sometimes delay, alleviate or halt the progression of breast cancer. Any known preventative or therapeutic treatment for alleviating or preventing the occurrence of breast cancer is prescribed and/or administered. For example, certain preventative treatments often are prescribed to subjects having a predisposition to breast cancer and where the subject is not diagnosed with breast cancer or is diagnosed as having symptoms indicative of early stage breast cancer (e.g., stage I). For subjects not diagnosed as having breast cancer, any preventative treatments known in the art can be prescribed and administered, which include selective hormone receptor modulators (e.g., selective estrogen receptor modulators (SERMs) such as tamoxifen, reloxifene, and toremifene); compositions that prevent production of hormones (e.g., aramotase inhibitors that prevent the production of estrogen in the adrenal gland, such as exemestane, letrozole, anastrozol, groserelin, and megestrol); other hormonal treatments (e.g., goserelin acetate and fulvestrant); biologic response modifiers such as antibodies (e.g., trastuzumab (herceptin/HER2)); surgery (e.g., lumpectomy and mastectomy); drugs that delay or halt metastasis (e.g., pamidronate disodium); and alternative/complementary medicine (e.g., acupuncture, acupressure, moxibustion, qi gong, reiki, ayurveda, vitamins, minerals, and herbs (e.g., astragalus root, burdock root, garlic, green tea, and licorice root)).

The use of breast cancer treatments are well known in the art, and include surgery, chemotherapy and/or radiation therapy. Any of the treatments may be used in combination to treat or prevent breast cancer (e.g., surgery followed by radiation therapy or chemotherapy). Examples of chemotherapy combinations used to treat breast cancer include: cyclophosphamide (Cytoxan), methotrexate (Amethopterin, Mexate, Folex), and fluorouracil (Fluorouracil, 5-Fu, Adrucil), which is referred to as CMF; cyclophosphamide, doxorubicin (Adriamycin), and fluorouracil, which is referred to as CAF; and doxorubicin (Adriamycin) and cyclophosphamide, which is referred to as AC.

As breast cancer preventative and treatment information can be specifically targeted to subjects in need thereof (e.g., those at risk of developing breast cancer or those that have early signs of breast cancer), provided herein is a method for preventing or reducing the risk of developing breast cancer in a subject, which comprises: (a) detecting the presence or absence of a polymorphic variation associated with breast cancer at a polymorphic site in a nucleotide sequence in a nucleic acid sample from a subject; (b) identifying a subject at risk of breast cancer, whereby the presence of the polymorphic variation is indicative of a risk of breast cancer in the subject; and (c) if such a risk is identified, providing the subject with information about methods or products to prevent or reduce breast cancer or to delay the onset of breast cancer. Also provided is a method of targeting information or advertising to a subpopulation of a human population based on the subpopulation being genetically predisposed to a disease or condition, which comprises: (a) detecting the presence or absence of a polymorphic variation associated with breast cancer at a polymorphic site in a nucleotide sequence in a nucleic acid sample from a subject; (b) identifying the subpopulation of subjects in which the polymorphic variation is associated with breast cancer; and (c) providing information only to the subpopulation of subjects about a particular product which may be obtained and consumed or applied by the subject to help prevent or delay onset of the disease or condition.

Pharmacogenomics methods also may be used to analyze and predict a response to a breast cancer treatment or a drug. For example, if pharmacogenomics analysis indicates a likelihood that an individual will respond positively to a breast cancer treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The methods described herein also are applicable to clinical drug trials. One or more polymorphic variants indicative of response to an agent for treating breast cancer or to side effects to an agent for treating breast cancer may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems. In certain embodiments, the agent for treating breast cancer described herein targets KLF12 or a target in the KLF12 pathway (e.g., Rho GTPase).

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1, 2 or 3; (ii) a polynucleotide sequence that is 90% or more identical to a nucleotide sequence set forth in SEQ ID NO: I, 2 or 3; (iii) a polynucleotide sequence that encodes a polypeptide having an amino acid sequence identical to or 90% or more identical to an amino acid sequence encoded by a nucleotide sequence set forth in SEQ ID NO: 1, 2 or 3; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with breast cancer at a polymorphic site in a nucleotide sequence in a nucleic acid sample from a subject; (b) the diagnostic/prognostic testing provider identifies the subpopulation of subjects in which the polymorphic variation is associated with breast cancer; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising Breast Cancer-Directed Molecules

Featured herein is a composition comprising a breast cancer cell and one or more molecules specifically directed and targeted to a nucleic acid comprising a KLF12 nucleotide sequence or a KLF12 polypeptide. Such directed molecules include, but are not limited to, a compound that binds to a KLF12 nucleic acid or a KLF12 polypeptide; a RNAi or siRNA molecule having a strand complementary to a KLF12 nucleotide sequence; an antisense nucleic acid complementary to an RNA encoded by a KLF12 DNA sequence; a ribozyme that hybridizes to a KLF12 nucleotide sequence; a nucleic acid aptamer that specifically binds a KLF12 polypeptide; and an antibody that specifically binds to a KLF12 polypeptide or binds to a KLF12 nucleic acid. In specific embodiments, the breast cancer directed molecule interacts with a KLF12 nucleic acid or polypeptide variant associated with breast cancer. In other embodiments, the breast cancer directed molecule interacts with a polypeptide involved in the KLF12 signal pathway, or a nucleic acid encoding such a polypeptide.

Compositions sometimes include an adjuvant known to stimulate an immune response, and in certain embodiments, an adjuvant that stimulates a T-cell lymphocyte response. Adjuvants are known, including but not limited to an aluminum adjuvant (e.g., aluminum hydroxide); a cytokine adjuvant or adjuvant that stimulates a cytokine response (e.g., interleukin (IL)-12 and/or γ-interferon cytokines); a Freund-type mineral oil adjuvant emulsion (e.g., Freund's complete or incomplete adjuvant); a synthetic lipoid compound; a copolymer adjuvant (e.g., TitreMax); a saponin; Quil A; a liposome; an oil-in-water emulsion (e.g., an emulsion stabilized by Tween 80 and pluronic polyoxyethlene/polyoxypropylene block copolymer (Syntex Adjuvant Formulation); TitreMax; detoxified endotoxin (MPL) and mycobacterial cell wall components (TDW, CWS) in 2% squalene (Ribi Adjuvant System)); a muramyl dipeptide; an immune-stimulating complex (ISCOM, e.g., an Ag-modified saponin/cholesterol micelle that forms stable cage-like structure); an aqueous phase adjuvant that does not have a depot effect (e.g., Gerbu adjuvant); a carbohydrate polymer (e.g., AdjuPrime); L-tyrosine; a manide-oleate compound (e.g., Montanide); an ethylene-vinyl acetate copolymer (e.g., Elvax 40W1,2); or lipid A, for example. Such compositions are useful for generating an immune response against a breast cancer directed molecule (e.g., an HLA-binding subsequence within a polypeptide encoded by a nucleotide sequence in SEQ ID NO: 1). In such methods, a peptide having an amino acid subsequence of a polypeptide encoded by a nucleotide sequence in SEQ ID NO: 1, 2 or 3 is delivered to a subject, where the subsequence binds to an HLA molecule and induces a CTL lymphocyte response. The peptide sometimes is delivered to the subject as an isolated peptide or as a minigene in a plasmid that encodes the peptide. Methods for identifying HLA-binding subsequences in such polypeptides are known (see e.g., publication WO02/20616 and PCT application US98/01373 for methods of identifying such sequences).

The breast cancer cell may be in a group of breast cancer cells and/or other types of cells cultured in vitro or in a tissue having breast cancer cells (e.g., a melanocytic lesion) maintained in vitro or present in an animal in vivo (e.g., a rat, mouse, ape or human). In certain embodiments, a composition comprises a component from a breast cancer cell or from a subject having a breast cancer cell instead of the breast cancer cell or in addition to the breast cancer cell, where the component sometimes is a nucleic acid molecule (e.g., genomic DNA), a protein mixture or isolated protein, for example. The aforementioned compositions have utility in diagnostic, prognostic and pharmacogenomic methods described previously and in breast cancer therapeutics described hereafter. Certain breast cancer molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al, J. Med. Chem.37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes alters expression and sometimes alters activity of a KLF12 polypeptide and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire coding strand in SEQ ID NO: 1, 2 or 3, or to a portion thereof or a substantially identical sequence thereof. In another embodiment, the antisense nucleic acid molecule is antisense to a “noncoding region” of the coding strand of a nucleotide sequence in SEQ ID NO: 1 (e.g., 5′ and 3′ untranslated regions).

An antisense nucleic acid can be designed such that it is complementary to the entire coding region of an mRNA encoded by a nucleotide sequence in SEQ ID NO: 1 (e.g., SEQ ID NO: 2), and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35,40, 45, 50, 55, 60, 65, 70, 75, 80, or more nucleotides in length. The antisense nucleic acids, which include the ribozymes described hereafter, can be designed to target a nucleotide sequence in SEQ ID NO: 1, 2 or 3, often a variant associated with breast cancer, or a substantially identical sequence thereof. Among the variants, minor alleles and major alleles can be targeted, and those associated with a higher risk of breast cancer are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are *-anomeric nucleic acid molecules. An *-anomeric nucleic acid molecule forms specific double-stranded hybrids with complementary RNA in which, contrary to the usual *-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a KLF12 nucleotide sequence can include one or more sequences complementary to such a nucleotide sequence, and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (see e.g., Cech et al. U.S. Pat. No.4,987,071; and Cech et al. U.S. Pat. No.5,116,742). Also, target mRNA sequences can be used to select a catalytic RNA having a specific ribonuclease activity from a pool of RNA molecules (see e.g., Bartel & Szostak, Science 261: 1411-1418 (1993)).

Breast cancer directed molecules include in certain embodiments nucleic acids that can form triple helix structures with a KLF12 nucleotide sequence or a substantially identical sequence thereof, especially one that includes a regulatory region that controls expression of a polypeptide. Gene expression can be inhibited by targeting nucleotide sequences complementary to the regulatory region of a KLF12 nucleotide sequence or a substantially identical sequence (e.g., promoter and/or enhancers) to form triple helical structures that prevent transcription of a gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Breast cancer directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol 2000 February;2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci U S A. 2001 August 14;98(17):9742-7 and Elbashir et al. Methods 2002 February;26(2):199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. patent application No. US2001000993183; Caplen et al Proc Natl Acad Sci U S A; and Abderrahmani et al. Mol Cell Biol 2001 Nov. 21(21):7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA.

When designing the siRNA molecules, the targeted region often is selected from a given DNA sequence beginning 50 to 100 nucleotides downstream of the start codon. See, e.g., Elbashir et al,. Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g.. 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc.

Antisense, ribozyme, RNAi and siRNA nucleic acids can be altered to form modified nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al, Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNA nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNA nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, e.g.; PCT Publication No. WO89/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988) ). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a nucleotide sequence of SEQ ID NO: 1, 2 or 3 or a substantially identical sequence thereof, two complementary regions one having a fluorophore and one a quencher such that the molecular beacon is useful for quantifying the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. 5,876,930.

Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

A full-length polypeptide or antigenic peptide fragment encoded by a KLF12 nucleotide sequence can be used as an immunogen or can be used to identify antibodies made with other immunogens, e.g., cells, membrane preparations, and the like. An antigenic peptide often includes at least 8 amino acid residues of the amino acid sequences encoded by a nucleotide sequence of SEQ ID NO: 1, 2 or 3, or substantially identical sequence thereof, and encompasses an epitope. Antigenic peptides sometimes include 10 or more amino acids, 15 or more amino acids, 20 or more amino acids, or 30 or more amino acids. Hydrophilic and hydrophobic fragments of polypeptides sometimes are used as immunogens.

Epitopes encompassed by the antigenic peptide are regions located on the surface of the polypeptide (e.g., hydrophilic regions) as well as regions with high antigenicity. For example, an Emini surface probability analysis of the human polypeptide sequence can be used to indicate the regions that have a particularly high probability of being localized to the surface of the polypeptide and are thus likely to constitute surface residues useful for targeting antibody production. The antibody may bind an epitope on any domain or region on polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Patent No. 4,816,567; Cabilly et al European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun el al., Proc. Natl. Acad. Sci. USA 84: 214-218 (1987); Nishimura et al., Canc. Res. 47: 999-1005 (1987); Wood et al.; Nature 314: 446449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 40534060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An antibody can be a single chain antibody. A single chain antibody (scFV) can be engineered (see, e.g., Colcher et al., Ann. N Y Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophospliamide, busulfan, dibromomannitol, streptozotocin, mitomycin C, and cis-dichliorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An antibody (e.g., monoclonal antibody) can be used to isolate target polypeptides by standard techniques, such as affinity chromatography or immunoprecipitation. Moreover, an antibody can be used to detect a target polypeptide (e.g., in a cellular lysate or cell supernatant) in order to evaluate the abundance and pattern of expression of the polypeptide. Antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an antibody can be utilized as a test molecule for determining whether it can treat breast cancer, and as a therapeutic for administration to a subject for treating breast cancer.

An antibody can be made by immunizing with a purified antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native polypeptide, only denatured or otherwise non-native polypeptide, or which bind both, as well as those having linear or conformational epitopes. Conformational epitopes sometimes can be identified by-selecting antibodies that bind to native but not denatured polypeptide. Also featured are antibodies that specifically bind to a polypeptide variant associated with breast cancer.

Screening Assays

Featured herein are methods for identifying a candidate therapeutic for treating breast cancer. The methods comprise contacting a test molecule with a target molecule in a system. A “target molecule” as used herein refers to a nucleic acid of SEQ ID NO: 1, 2 or 3, a substantially identical nucleic acid thereof, or a fragment thereof, and an encoded polypeptide of the foregoing. The method also comprises determining the presence or absence of an interaction between the test molecule and the target molecule, where the presence of an interaction between the test molecule and the nucleic acid or polypeptide identifies the test molecule as a candidate breast cancer therapeutic. The interaction between the test molecule and the target molecule may be quantified.

Test molecules and candidate therapeutics include, but are not limited to, compounds, antisense nucleic acids, siRNA molecules, ribozymes, polypeptides or proteins encoded by a KLF12 nucleic acids, or a substantially identical sequence or fragment thereof, and immunotherapeutics (e.g., antibodies and HLA-presented polypeptide fragments). A test molecule or candidate therapeutic may act as a modulator of target molecule concentration or target molecule function in a system. A “modulator” may agonize (i.e., up-regulates) or antagonize (i.e., down-regulates) a target molecule concentration partially or completely in a system by affecting such cellular functions as DNA replication and/or DNA processing (e.g., DNA methylation or DNA repair), RNA transcription and/or RNA processing (e.g., removal of intronic sequences and/or translocation of spliced mRNA from the nucleus), polypeptide production (e.g., translation of the polypeptide from mRNA), and/or polypeptide post-translational modification (e.g., glycosylation, phosphorylation, and proteolysis of pro-polypeptides). A modulator may also agonize or antagonize a biological function of a target molecule partially or completely, where the function may include adopting a certain structural conformation, interacting with one or more binding partners, ligand binding, catalysis (e.g., phosphorylation, dephosphorylation, hydrolysis, methylation, and isomerization), and an effect upon a cellular event (e.g., effecting progression of breast cancer).

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on test molecule, where the effect sometimes is binding between the test molecule and the target molecule, and sometimes is an observable change in cells, tissue, or organism.

There are many standard methods for detecting the presence or absence of interaction between a test molecule and a KLF12 nucleic acid or polypeptide. For example, titrametric, acidimetric, radiometric, NMR, monolayer, polarographic, spectrophotometric, fluorescent, and ESR assays probative of KLF12 function may be utilized.

An interaction can be determined by labeling the test molecule and/or the KLF12 molecule, where the label is covalently or non-covalently attached to the test molecule or KLF12 molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. Also, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and KLF12 (McConnell, H. M. et al., Science 257: 1906-1912 (1992)).

In cell-based systems, cells often include a KLF12 nucleic acid or polypeptide or variants thereof and are often of mammalian origin, although the cell can be of any origin. Whole cells, cell homogenates, and cell fractions (e.g., cell membrane fractions) can be subjected to analysis. Where interactions between a test molecule with a KLF12 polypeptide or variant thereof are monitored, soluble and/or membrane bound forms of the polypeptide or variant may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between two molecules can also be detected by monitoring fluorescence energy transfer (FET) (see, for example, Lakowicz et al., U.S. Pat. No. 5,631,169; Stavrianopoulos et al. U.S. Pat. No. 4,868,103). A fluorophore label on a first, “donor” molecule is selected such that its emitted fluorescent energy is absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a KLF12 molecule can be effected by using real-time Biomolecular Interaction Analysis (BIA) (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the KLF12 molecule or test molecules are anchored to a solid phase. The KLF12 molecule/test molecule complexes anchored to the solid phase can be detected at the end of the reaction. The target KLF12 molecule is often anchored to a solid surface, and the test molecule, which is not anchored, can be labeled, either directly or indirectly, with detectable labels discussed herein.

It may be desirable to immobilize a KLF12 molecule, an anti-KLF12 antibody, or test molecules to facilitate separation of complexed from uncomplexed forms of KLF12 molecules and test molecules, as well as to accommodate automation of the assay. Binding of a test molecule to a KLF12 molecule can be accomplished in any vessel suitable for containing the reactants. Examples of such vessels include microtiter plates, test tubes, and micro-centrifuge tubes. In one embodiment, a fusion polypeptide can be provided which adds a domain that allows a KLF12 molecule to be bound to a matrix. For example, glutathione-S-transferase/KLF12 fusion polypeptides or glutathione-S-transferase/target fusion polypeptides can be adsorbed onto glutathione sepharose beads (Sigma Chemical, St. Louis, Mo.) or glutathione derivatized microtiter plates, which are then combined with the test compound or the test compound and either the non-adsorbed target polypeptide or KLF12 polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of KLF12 binding or activity determined using standard techniques.

Other techniques for immobilizing a KLF12 molecule on matrices include using biotin and streptavidin. For example, biotinylated KLF12 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with KLF12 polypeptide or test molecules but which do not interfere with binding of the KLF12 polypeptide to its test molecule. Such antibodies can be derivatized to the wells of the plate, and unbound target or KLF12 polypeptide trapped in the wells by antibody conjugation. Methods for detecting such complexes, in addition to those described above for the GST-immobilized complexes, include immunodetection of complexes using antibodies reactive with the KLF12 polypeptide or target molecule, as well as enzyme-linked assays which rely on detecting an enzymatic activity associated with the KLF12 polypeptide or test molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci August; 18(8): 284-7 (1993)); chromatography (gel filtration chromatography, ion-exchange chromatography); electrophoresis (see, e.g., Ausubel et al., eds. Current Protocols in Molecular Biology, J. Wiley: New York (1 999)); and immunoprecipitation (see, for example, Ausubel, F. et al., eds. Current Protocols in Molecular Biology, J. Wiley: New York (1999)). Such resins and chromatographic techniques are known to one skilled in the art (see, e.g., Heegaard, J. Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of KLF12 expression are identified. For example, a cell or cell free mixture is contacted with a candidate compound and the expression of KLF12 mRNA or polypeptide evaluated relative to the level of expression of KLF12 mRNA or polypeptide in the absence of the candidate compound. When expression of KLF12 mRNA or polypeptide is greater in the presence of the candidate compound than in its absence, the candidate compound is identified as a stimulator of KLF12 mRNA or polypeptide expression. Alternatively, when expression of KLF12 mRNA or polypeptide is less (statistically significantly less) in the presence of the candidate compound than in its absence, the candidate compound is identified as an inhibitor of KLF12 mRNA or polypeptide expression. The level of KLF12 mRNA or polypeptide expression can be determined by methods described herein for detecting KLF12 mRNA or polypeptide. A similar assay is further described in Example 4.

In another embodiment, binding partners that interact with a KLF12 molecule are detected. The KLF12 molecules can interact with one or more cellular or extracellular macromolecules, such as polypeptides, in vivo, and these molecules that interact with KLF12 molecules are referred to herein as “binding partners.” Molecules that disrupt such interactions can be useful in regulating the activity of the target gene product. For example, in the case of KLF12, molecules that block the ability of KLF12 to inhibit the expression of secondary regulatory genes that play a role in oncogenesis, such as AP-2, can be detected and subsequently used as anti-cancer agents. Such molecules can include, but are not limited to molecules such as antibodies, peptides, siRNA and small molecules. Target genes/products for use in this embodiment often are the KLF12 genes herein identified. In an alternative embodiment, provided is a method for determining the ability of the test compound to modulate the activity of a KLF12 polypeptide through modulation of the activity of a downstreain-effector of a KLF12 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases where it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No.4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Also, binding partners of KLF12 molecules can be identified in a two-hybrid assay or three-hybrid assay (see, e.g., U.S. Pat. No.5,283,317; Zervos et al., Cell 72:223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300), to identify other polypeptides, which bind to or interact with KLF12 (“KLF12-binding polypeptides” or “KLF12-bp”) and are involved in KLF12 activity. Such KLF12-bps can be activators or inhibitors of signals by the KLF12 polypeptides or KLF12 targets as, for example, downstream elements of a KLF12-mediated signaling pathway.

A two-hybrid system is based on the modular nature of most transcription factors, which consist of separable DNA-binding and activation domains. Briefly, the assay utilizes two different DNA constructs. In one construct, the gene that codes for a KLF12 polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. (Alternatively the: KLF12 polypeptide can be the fused to the activator domain.) If the “bait” and the “prey” polypeptides are able to interact, in vivo, forming a KLF12-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the KLF12 polypeptide.

Candidate therapeutics for treating cancer are identified from a group of test molecules that interact with a KLF12 nucleic acid or polypeptide. Test molecules are normally ranked according to the degree with which they interact or modulate (e.g., agonize or antagonize) DNA replication and/or processing, RNA transcription and/or processing, polypeptide production and/or processing, and/or function of KLF12 molecules, for example, and then top ranking modulators are selected. In an embodiment the candidate therapeutic (i.e., test molecule) acts as a KLF12 antagonist. In another embodiment, the candidate therapeutic is a siRNA molecule capable of inhibiting gene expression of KLF12 or, optionally, any of its transcripts. Also, pharmacogenomic information described herein can determine the rank of a modulator. Candidate therapeutics often are formulated for administration to a subject.

Therapeutic Treatments

Formulations or pharmaceutical compositions often include in combination with a pharmaceutically acceptable carrier, a compound, an antisense nucleic acid, a siRNA molecule capable of inhibiting the expression of KLF12 or, optionally, any of its transcripts, a ribozyme, an antibody, a binding partner that interacts with a KLF12 polypeptide, a KLF12 nucleic acid, or a fragment thereof. The formulated molecule may be one that is identified by a screening method described herein. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium sfearate or Sterotes; a glidant such as colloidal silicon dioxide; a sweetening agent. such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of rnanufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, sodium chloride sometimes are included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation often utilized are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules often lies within a range of circulating concentrations that include the ED₅₀ with little or no toxicity. The dosage may vary within this range depending upon the dosage form employed and the route of administration utilized. For any molecules used in the methods described herein, the therapeutically effective dose can be estimated initially from cell culture assays. A dose may be formulated in animal models to achieve a circulating plasma concentration range that includes the IC₅₀ (i:e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment, or sometimes can include a series of treatments.

With regard to polypeptide formulations, featured herein is a method for treating cancer in a subject, which comprises contacting one or more cells in the subject with a first KLF12 polypeptide, where genomic DNA in the subject comprises a second KLF12 nucleic acid having one or more polymorphic variations associated with cancer, and where the first KLF12 polypeptide comprises fewer polymorphic variations associated with cancer than the first KLF12 polypeptide. The first and second KLF12 polypeptides are encoded by a nucleic acid which comprises a nucleotide sequence selected from the group consisting of the nucleotide sequence of SEQ. ID NO: 1; a nucleotide sequence which encodes a polypeptide consisting of an amino acid sequence set forth in FIGS. 3A or 3B; and a nucleotide sequence which encodes a polypeptide that is 90% identical to an amino acid sequence set forth in FIGS. 3A or 3B. The second KLF12 polypeptide also may be encoded by a fragment of the foregoing nucleic acids comprising the one or more polymorphic variations. The subject is often a human.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

KLF12 nucleic acid molecules can be inserted into vectors and used in gene therapy methods for treating cancer. Featured herein is a method for treating cancer in a subject, which comprises contacting one or more cells in the subject with a first KLF12 nucleic acid, where genomic DNA in the subject comprises a second KLF12 nucleic acid comprising one or more polymorphic variations associated with cancer, and where the first KLF12 nucleic acid comprises fewer polymorphic variations associated with cancer. The first and second KLF12 nucleic acids often comprise a nucleotide sequence selected from the group consisting of the nucleotide sequence of SEQ ID NO: 1; a nucleotide sequence which encodes a polypeptide consisting of an amino acid sequence set forth in FIGS. 3A or 3B; and a nucleotide sequence which encodes a polypeptide that is 90% identical to an amino acid sequence set forth in FIGS. 3A or 3B. The second KLF12 nucleic acid may also be a fragment of the foregoing comprising one or more polymorphic variations. For example, the second KLF12 nucleic acid, or a fragment thereof, may contain an adenine at position 96535 of SEQ ID NO: 1. The subject is often a human.

Gene therapy vectors can be delivered to a subject by, for example, intravenous injection, local administration (see U.S. Pat. No. 5,328,470) or by stereotactic injection (see e.g., Chen et al., (1994) Proc. Natl. Acad Sci. USA 91:3054-3057). Pharmaceutical preparations of gene therapy vectors can include a gene therapy vector in an acceptable diluent, or can-comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Pharmaceutical compositions can be included in a container, pack, or dispenser together with instructions for administration.

Pharmaceutical compositions of active ingredients can be administered by any of the paths described herein for therapeutic and prophylactic methods for treating cancer. With regard to both prophylactic and therapeutic methods of treatment, such treatments may be specifically tailored or modified, based on knowledge obtained from pharmacogenomic analyses described herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Administration of a prophylactic agent can occur prior to the manifestation of symptoms characteristic of the KLF12 aberrance, such that a disease or disorder is prevented or, alternatively, delayed in its progression. Depending on the type of KLF12 aberrance, for example, a KLF12 molecule, KLF12 agonist, or KLF12 antagonist agent can be used for treating the subject. The appropriate agent can be determined based on screening assays described herein.

As discussed, successful treatment of KLF12 disorders can be brought about by techniques that serve to inhibit the expression or activity of target gene products. For example, compounds (e.g., an agent identified using an assays described above) that exhibit negative modulatory activity can be used to prevent and/or treat cancer. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances where the target gene encodes an extracellular polypeptide, normal target gene polypeptide often is co-administered into the cell or tissue to maintain the requisite level of cellular or tissue target gene activity.

Another method by which nucleic acid molecules may be utilized in treating or preventing a disease characterized by KLF12 expression is through the use of aptamer molecules specific for KLF12 polypeptide. Aptamers are nucleic acid molecules having a tertiary structure which permits them to specifically bind to polypeptide ligands (see, e.g., Osborne, et al., Curr. Opin. Chem. Biol 1(1): 5-9 (1997); and Patel, D. J., Curr. Opin. Chem. Biol. June;1(1): 32-46 (1997)). Since nucleic acid molecules may in many cases be more conveniently introduced into target cells than therapeutic polypeptide molecules may be, aptamers offer a method by which KLF12 polypeptide activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of KLF12 disorders. For a description of antibodies, see the Antibody section above.

In circumstances where injection of an animal or a human subject with a KLF12 polypeptide or epitope for stimulating antibody production is harmful to the subject, it is possible to generate an immune response against KLF12 through the use of anti-idiotypic antibodies (see, for example, Herlyn, D., Ann. Med.;31(1): 66-78 (1999); and Bhattacharya-Chatterjee & Foon, Cancer Treat. Res.; 94: 51-68 (1998)). If an anti-idiotypic antibody is introduced into a mammal or human subject, it should stimulate the production of anti-anti-idiotypic antibodies, which should be specific to the KLF12 polypeptide. Vaccines directed to a disease characterized by KLF12 expression may also be generated in this fashion.

In instances where the target antigen is intracellular and whole antibodies are used, internalizing antibodies may be utilized. Lipofectin or liposomes can be used to deliver the antibody or a fragment of the Fab region that binds to the target antigen into cells. Where fragments of the antibody are used, the smallest inhibitory fragment that binds to the target antigen often is utilized. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90. 7889-7893 (1993)).

KLF12 molecules and compounds that inhibit target gene expression, synthesis and/or activity can be administered to a patient at therapeutically effective doses to prevent, treat or ameliorate KLF12 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices often are utilized. While compounds that exhibit toxic side effects can be used, care should be taken to design a delivery system that targets such compounds to the site of affected tissue in order to minimize potential damage to unifected cells and, thereby, reduce side effects.

Data obtained from cell culture assays and animal studies can be used in formulating a range of dosage for use in humans. The dosage of such compounds often lies within a range of circulating concentrations that include the ED₅₀ with little or no toxicity. The dosage can vary within this range depending upon the dosage form employed and the route of administration utilized. For any compound used in a method described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. The compound which is able to modulate KLF12 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates the expression or activity. of KLF12 can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al., Analytical Chemistry 67: 2142-2144 (1995).

Provided herein are methods of modulating KLF12 expression or activity for therapeutic purposes. Accordingly, in an exemplary embodiment, the modulatory method involves contacting a cell with a KLF12 or agent that modulates one or more of the activities of KLF12 polypeptide activity associated with the cell. An agent that modulates KLF12 polypeptide activity can be an agent as described herein, such as a nucleic acid or a polypeptide, a naturally-occurring target molecule of a KLF12 polypeptide (e.g., a KLF12 substrate or receptor), a KLF12 antibody, a KLF12 agonist or antagonist, a peptidomimetic of a KLF12 agonist or antagonist, or other small molecule.

In one embodiment, the agent stimulates one or more KLF12 activities. In another embodiment, the agent inhibits one or more KLF12 activities. Examples of such inhibitory agents include antisense KLF12 nucleic acid molecules, anti-KLF12 antibodies, KLF12 inhibitors, siRNA molecules capable of inhibiting the expression of KLF12 or, optionally, any KLF12 transcripts, and competitive inhibitors that target secondary regulatory genes, such as AP-2, that are regulated by KLF12. These modulatory methods can be performed in vitro (e.g., by culturing the cell with the agent) or, alternatively, in vivo (e.g., by administering the agent to a subject). As such, provided are methods of treating an individual afflicted with a disease or disorder characterized by aberrant or unwanted expression or activity of a KLF12 polypeptide or nucleic acid molecule. In one embodiment, the method involves administering an agent (e.g., an agent identified by a screening assay described herein), or combination of agents that modulates (e.g., upregulates or downregulates) KLF12 expression or activity. In an embodiment, the method involves administering an agent (e.g., an agent identified by a screening assay described herein or an siRNA molecule capable: of inhibiting the expression of KLF12, or any of its transcripts), or a combination of agents that inhibits KLF12 expression or activity (e.g., a KLF12 activity may include inhibiting the expression of secondary regulatory genes, such as AP-2, that play a role in oncogenesis). In another embodiment, the method involves administering a KLF12 polypeptide or nucleic acid molecule as therapy to compensate for reduced, aberrant, or unwanted KLF12 expression or activity.

Stimulation of KLF12 activity is desirable in situations in which KLF12 is abnormally downregulated and/or in which increased KLF12 activity is likely to have a beneficial effect. For example, stimulation of KLF12 activity is desirable in situations in which a KLF12 is downregulated and/or in which increased KLF12 activity is likely to have a beneficial effect. Likewise, inhibition of KLF12 activity is desirable in situations in which KLF12 is abnormally upregulated and/or in which decreased KLF12 activity is likely to have a beneficial effect.

Methods of Treatment

It is known that AP-2a transactivates the cyclin dependent kinase inhibitor, p21WAF1/CIP1 promoter, resulting in G1 and G2 cell cycle arrest. This role of AP-2a is relevant to its reported tumor suppressor activity in cancer, since the tumor suppressor function of AP-2a is facilitated by release of repression driven by transient inhibition of KLF12 with the transfected siKLF1 reagent (see Example 7). Regardless of the precise mechanism, KFL12 overexpression is implicated in the regulation of the growth pattern of selected tumor cell lines (see Example 8). Though not wishing to be bound or limited by any proposed mechanism for the observed effect, it is expected that inhibiting the overexpression of KLF12 and thereby increasing the expression of AP-2a results in growth arrest and cell death via the cell cycle inhibitor p21 WAF1/CIP1 or a similar inhibitor.

Alternatively, it is possible that KLF12 has an oncogenic role in cancer similar to a highly homologous protein, Wilm's tumor 1 protein (WT1) (Zapata-Benavides P, et al. Biochem Biophys Res Commun. 2002 July 26;295(4):784-90). Zapata-Benavides et al. used antisense molecules to knock down WT1 expression and saw decreased proliferation in several breast cancer cell lines. WT1 is very homologous to KLF12, with 4 Kruppel-type zinc fingers, and both proteins share a highly related consensus DNA binding sequence (Imhof et al. Mol Cell Biol 1999 January;19(1):194-204)) and (Roth et al. Genomics 63:384-390 (2000)). Therefore, it is believed WT1 and KLF12 share similar functions as human transcription repressors.

Thus, provided are methods for identifying a risk of cancer in an individual as described herein and, if a genetic predisposition is identified, treating that individual to delay or reduce or prevent the development of cancer. Such a procedure can be used to treat cancer. Optionally, treating an individual for cancer may include inhibiting cellular proliferation, inhibiting metastasis, inhibiting invasion, or preventing tumor formation or growth as defined herein. Suitable treatments to prevent or reduce or delay cancer focus on inhibiting additional cellular proliferation, inhibiting metastasis, inhibiting invasion, and preventing further tumor formation or growth. In the case of breast cancer, treatment usually includes surgery followed by radiation therapy. Surgery may be a lumpectomy or a mastectomy (e.g., total, simple or radical). Even if the doctor removes all of the cancer that can be seen at the time of surgery, the patient may be given radiation therapy, chemotherapy, or hormone therapy after surgery to try to kill any cancer cells that may be left. Radiation therapy is the use of x-rays or other types of radiation to kill cancer cells and shrink tumors. Radiation therapy may use external radiation (using a machine outside the body) or internal radiation. Chemotherapy is the use of drugs to kill cancer cells. Chemotherapy may be taken by mouth, or it may be put into the body by inserting a needle into a vein or muscle. Hormone therapy often focuses on estrogen and progesterone, which are hormones that affect the way some cancers grow. If tests show that the cancer cells have estrogen and progesterone receptors (molecules found in some cancer cells to which estrogen and progesterone will attach), hormone therapy is used to block the way these hormones help the cancer grow. Hormone therapy with tamoxifen is often given to patients with early stages of breast cancer and those with metastatic breast cancer. Other types of treatment being tested in clinical trials include sentinel lymph node biopsy followed by surgery and high-dose chemotherapy with bone marrow transplantation and peripheral blood stem cell transplantation. Any preventative/therapeutic treatment known in the art may be prescribed and/or administered, including, for example, surgery, chemotherapy and/or radiation treatment, and any of the treatments may be used in combination with one another to treat or prevent breast cancer (e.g., surgery followed by radiation therapy).

Also provided are methods of preventing or treating cancer comprising providing an individual in need of such treatment with a KLF12 inhibitor that reduces or inhibits the overexpression of mutant KLF12 (e.g., a KLF12 polynucleotide with an allele that is associated with cancer set forth in Table 10 and Table 12). Included herein are methods of reducing or blocking the expression of KLF12 comprising providing or administering to individuals in need of reducing or blocking the expression of KLF12 a pharmaceutical or physiologically acceptable composition comprising a molecule capable of inhibiting expression of KLF12, e.g., a siRNA molecule. Also included herein are methods of reducing or blocking the expression of secondary regulatory genes regulated by KLF12, such as AP-2, that play a role in oncogenesis which comprises introducing competitive inhibitors that target KLF12's effect on these regulatory genes or that block the binding of positive factors, e.g., KLF12, necessary for the expression of these regulatory genes.

The examples set forth below are intended to illustrate but not limit the invention.

### EXAMPLES

In the following studies a group of subjects were selected according to specific parameters relating to breast cancer. Nucleic acid samples obtained from individuals in the study group were subjected to genetic analysis, which identified associations between breast cancer and polymorphisms in the KLF12 gene and regions surrounding the gene on chromosome three. Methods are described for producing KLF12 polypeptide and KLF12 polypeptide variants in vitro or in vivo, KLF12 nucleic acids or polypeptides and variants thereof are utilized for screening test molecules for those that interact with KLF12 molecules. Test molecules identified as interactors with KLF12 molecules and KLF12 variants are further screened in vivo to determine whether they treat breast cancer. Also, methods are described for comparing the expression of KLF12 in cancer and non-cancer cells, producing siRNA molecules capable of inhibiting KLF12 expression, measuring the effect of siRNA molecules that target KLF12 on cellular proliferation, and screening for KLF12 inhibitors.

### Example 1

### Samples and Pooling Strategies

Sample Selection

Blood samples were collected from individuals diagnosed with breast cancer, which were referred to as case samples. Also, blood samples were collected from individuals not diagnosed with breast cancer as gender and age-matched controls. All of the samples were of German/German descent. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. 2 ml of protein precipitation was added to the cell lysate. The mixtures were vortexed vigorously at high speed for 20 sec to mix the protein precipitation solution uniformly-with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (often 1:80, 1:160, 1:320, and 1:640 dilutions) so that it was within the measurable range of standards. 125 μl of diluted DNA was transferred to a clear U-bottom microtiter plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtiter plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/μl were re-measured for conformation. Samples having measured DNA concentrations of 20 ng/μl or less were re-measured for confirmation.

Pooling Strategies

Samples were placed into one of two groups based on disease status. The two groups were female case groups and female control groups. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool,each individual provided 125 pg of genomic DNA. Inclusion or exclusion of samples for a pool was based upon the following criteria: the sample was derived from an individual characterized as Caucasian; the sample was derived from an individual of German paternal and maternal descent; the database included relevant phenotype information for the individual; case samples Were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy). Samples that met these criteria were added to appropriate pools based on gender and disease status.

The selection process yielded the pools set forth in Table 2, which were used in the studies that follow:

TABLE 2

| | Female CASE | Female CONTROL |
|---|---|---|
| Pool size | 272 | 276 |
| (Number) | | |
| Pool Criteria | case | control |
| (ex: case/control) | | |
| Mean Age | 59.6 | 55.4 |
| (ex: years) | | |

### Example 2

### Association of Polymorphic Variants with Breast Cancer

A whole-genome screen was performed to identify particular SNPs associated with occurrence of breast cancer. As described in Example. 1, two sets of samples were utilized, which included samples from female individuals having breast cancer (breast cancer cases) and samples from female individuals not having cancer (female controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with breast cancer when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p value was calculated to determine whether the case and control groups had statistically significantly differences in allele frequencies for a particular SNP. When the genotyping results agreed with the original allelotyping results, the SNP disease association was considered validated at the genetic level.

It was discovered that females having a cytosine at position 96535 of SEQ ID NO: 1 were predisposed to breast cancer. This represents the incident SNP. Subsequently, proximal SNPs in and around the KLF12 gene were identified and allelotyped and a subset of the proximal SNPs were associated with breast cancer occurance. See, Example 3, “Identification of Proximal Polymorphic Variants in and around the KLF12 Gene”.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensembl genes; and they are located in Genomatix promoter predictions. SNPs in the set also were selected on the basis of even spacing across the genome, as depicted in Table 2.

A case-control study design using a whole genome association strategy involving approximately 28,000 single nucleotide polymorphisms (SNPs) was employed. Approximately 25,000 SNPs were evenly spaced in gene-based regions of the human genome with a median inter-marker distance of about 40,000 base pairs. Additionally, approximately 3,000 SNPs causing amino acid substitutions in genes described in the literature as candidates for various diseases were used. The case-control study samples were of female German origin (German paternal and maternal descent) 548 individuals were equally distributed in two groups (female controls and female cases). The whole genome association approach was first conducted on 2 DNA pools representing the 2 groups. Significant markers were confirmed by individual genotyping.

TABLE 3

| General Statistics | | Spacing Statistics | | |
|---|---|---|---|---|
| Total # of SNPs | 25,488 | Median | 37,058 | bp |
| # of Exonic SNPs | >4,335 (17%) | Minimum* | 1,000 | bp |
| # SNPs with refSNP ID | 20,776 (81%) | Maximum* | 3,000,000 | bp |
| Gene Coverage | >10,000 | Mean | 122,412 | bp |
| Chromosome Coverage | All | Std | 373,325 | bp |
| | | Deviation | | |

*Excludes outliers

Allelotyping and Genotyping Results

The genetic studies summarized above and described in more detail below identified an allelic variant associated with breast cancer, set forth in Table 4.

TABLE 4

| dbSNP | Position in SEQ | Allele | Cancer Associated |
|---|---|---|---|
| rs# | ID NO: 1 | Variants | Allele |
| rs1011058 | 96535 | C/T | C |

Assay for Verifying, Allelotyping, and Genotyping SNPs

A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer was used to genotype the polymorphism. Other primer design software could be used or one of ordinary skill in the art could manually design primers based on his or her knowledge of the relevant factors and considerations in designing such primers. Table 5 shows PCR primers and Table 6 shows extension primers used for analyzing polymorphisms. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 5
PCR Primers

| Refer- | | |
|---|---|---|
| ence | Forward | Reverse |
| SNP ID | PCR primer | PCR primer |
| rs | TGCAACACTCTGGTTACTTC | GCTAACTCCCAAATTGTACC |
| 1011058 | | |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No. 6,258,538, the content of which is hereby incorporated by reference. In Table 6, ddNTPs are shown and the fourth nucleotide not shown is the dNTP.

TABLE 6

| | | |
|---|---|---|
| Reference | | Term |
| SNP ID | Extend Probe | Mix |

| | | |
|---|---|---|
| rs1011058 | AACACTCTGGTTACTTCTTATTT | ACG |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP® (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

Variations identified in or around the KLF12 gene are represented by SEQ ID NO: 1 at position 96535. Minor allelic frequencies for this and all polymorphisms were verified as being 10% or greater by determining the allelic frequencies using the extension assay described above in a group of samples isolated from 92 individuals originating from the state of Utah in the United States, Venezuela and France (Coriell cell repositories).

Genotyping results are shown for female pools in Table 7. In the subsequent tables, “AF” refers to allelic frequency; and “F case” and “F control” refer to female case and female control groups, respectively.

TABLE 7

| Reference | AF | AF | | |
|---|---|---|---|---|
| SNP ID | F case | F control | p-value | Odds Ratio |
| rs1011058 | T = 0.819 | T = 0.891 | 0.0013 | 1.8 |
| | C = 0.181 | C = 0.109 | | |

As can be seen in Table 6, a cytosine at position 96535 were more common in the female breast cancer group. Genotyping results were considered significant with a calculated p-value of less than 0.05 for genotype results.

Odds ratio results are shown in Table 6. An odds ratio is an unbiased estimate of relative risk which can be obtained from most case-control studies. Relative risk (RR) is an estimate of the likelihood of disease in the exposed group (susceptibility allele or genotype carriers) compared to the unexposed group (not carriers). It can be calculated by the following equation:
RR=I_{A}/Iₐ
I_{A} is the incidence of disease in the A carriers and Iₐ is the incidence of disease in the non-carriers.
RR>1 indicates the A allele increases disease susceptibility.
RR<1 indicates the a allele increases disease susceptibility.

For example, RR=1.5 indicates that carriers of the A allele have 1.5 times the risk of disease than non-carriers, i.e., 50% more likely to get the disease.

Case-control studies do not allow the direct estimation of I_{A} and Iₐ therefore relative risk cannot be directly estimated. However, the odds ratio (OR) can be calculated using the following equation:
OR=(n_{DA}n_{da})/(n_{dA}n_{Da})=p_{DA}(1−p_{dA})/p_{dA}(1−p_{DA}), or
OR=((case f)/(1−case f))/((control f)/(1−control f)), where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has on the development of breast cancer, thus possessing that particular allele translates to having a higher risk of developing breast cancer.

### Example 3

### Identification of Proximal Polymorphic Variants in and around the KLF12 Gene

Polymorphic variants proximal to the incident SNP were identified and allelotyped as described in Example 2. The polymorphic variants represent intronic, intragenic and exonic SNPs that fall 50 kb 5′ and 50 kb 3′ of the incident SNP (rs1011058).

For each polymorphism, SpectroDESIGNER™ software (Sequenom, Inc.) was used to generate a set of PCR primers and a MassEXTEND™ primer was used to genotype the polymorphism. Other primer design software could be used or one of ordinary skill in the art could manually design primers based on his or her knowledge of the relevant factors and considerations in designing such primers. Table 8 shows PCR primers and Table 9 shows extension primers used for allelotyping the polymorphisms.

TABLE 8
PCR Primers

| SNP | | |
|---|---|---|
| Reference | Forward PCR primer | Reverse PCR primer |
| 3812851 | ACGTTGGATGCTGACTCCAAGAGTCAGTAA | ACGTTGGATGCACCTGATACTAGGTAATTG |
| 3812850 | ACGTTGGATGCAGCAAATGCTTGGTGATAC | ACGTTGGATGATCAGGTGATATGTGTCTGC |
| 2325555 | ACGTTGGATGCTCTCAGAGCCTTTCTGAAG | ACGTTGGATGTACAGTCACGTATTAGTGCC |
| 1535802 | ACGTTGGATGTTTCAAAACGCCCTTTCATC | ACGTTGGATGGGGTCAATACTACCTATTGTG |
| 1535801 | ACGTTGGATGGGGTCTTTTCAAGATAGGCC | ACGTTGGATGCAGTCACCTGCTTTTTGCTC |
| 718569 | ACGTTGGATGGAAGTCATCATTACCATGGC | ACGTTGGATGCTGTAGTTTAGTGGTGCTTC |
| 2325556 | ACGTTGGATGCAACACTCTTAGGTTTCAAGG | ACGTTGGATGCTCTTAGTTATCGAGTAGCC |
| 2274086 | ACGTTGGATGCTGCAGGGTGGTGTTACATT | ACGTTGGATGGAGGAGGTAAAGTCATTAAAG |
| 1886235 | ACGTTGGATGCACATTTGGCTCAAATGCAG | ACGTTGGATGCTACCCTTCACATTATAAACC |
| 1886234 | ACGTTGGATGTCAAGGGCGTGCAATTGTAG | ACGTTGGATGCAGAGGAATCTGCACAACAC |
| 2325558 | ACGTTGGATGGCCTTTTTTTAAGTATGGGA | ACGTTGGATGAGGTGAGATGATTAGGCATG |
| 2325559 | ACGTTGGATGAAGTAATACAGCCACAGTTC | ACGTTGGATGAAAAGTGGGCTCAATATCTG |
| 2325560 | ACGTTGGATGGGTTAACTTTGTGGAATGGAG | ACGTTGGATGCGATTCTTTAGAGAAGCGAT |
| 3764134 | ACGTTGGATGAGACTGTATACTCACCAAGG | ACGTTGGATGCTCTGTCTACAGGGTACATC |
| 3764133 | ACGTTGGATGTGGGAGTCTTCAGAGTGAAC | ACGTTGGATGTTCCTACGCCTGCAAATCTG |
| 2025426 | ACGTTGGATGAGAGAATACCATCACTCTGG | ACGTTGGATGATCAAGATGTTGCATCCCAC |
| 2025425 | ACGTTGGATGAGCACAGTAGGTAAGACTGC | ACGTTGGATGCTATCCAGGTAATTCCAGGG |
| 1324059 | ACGTTGGATGGTCTTGGCTGCTGAGTAAAG | ACGTTGGATGCTCAACTTCTCAGCAGCAAC |
| 2325561 | ACGTTGGATGAAATCCTTTGATGGCTGTCC | ACGTTGGATGAGAGGGAAAGGTTTAGCGAG |
| 2875666 | ACGTTGGATGAAATCCGACTGTGCCAGTTC | ACGTTGGATGTACAGGTGTCGTGTGTTGTG |
| 1408248 | ACGTTGGATGCCAATACAAAGTAGGCACAC | ACGTTGGATGTGCAGCAACACATCTTTAGG |
| 1324058 | ACGTTGGATGGCTACTAGACTGTTTCTCTG | ACGTTGGATGCAAGTCTTCATCGTAGTCAC |
| 1011059 | ACGTTGGATGGGCATTGACAGGCTAAATGC | ACGTTGGATGGGTACAATTTGGGAGTTAGC |
| 1011058 | ACGTTGGATGTGCAACACTCTGGTTACTTC | ACGTTGGATGGCTAACTCCCAAATTGTACC |
| 2325563 | ACGTTGGATGCCACCAGTTAGTTCTAGATC | ACGTTGGATGGCAATGCAACAACATGCAAG |
| 2325564 | ACGTTGGATGTACAGAGCTTGACAAAAGGC | ACGTTGGATGCTACAAAATGAGAGGTTAATC |
| 879800 | ACGTTGGATGTCTTCTTCAGTTGGTCAGTG | ACGTTGGATGAATAACAAGTGCAGAGGCCC |
| 2325565 | ACGTTGGATGCACCCCCTCCACTAGAATAT | ACGTTGGATGGCACTGTATCCATCAGACAC |
| 255595 | ACGTTGGATGCCCATAATCCTGGCACATTA | ACGTTGGATGTGGTAGTCTCTGAAGTGAGG |
| 2875667 | ACGTTGGATGTGTTTCATTGCACACTTCCC | ACGTTGGATGATGCCAGGACCCATCAATAG |
| 2325566 | ACGTTGGATGAACTATTGATGGGTCCTGGC | ACGTTGGATGGGAGATGCCAGATTCATTAC |
| 2325567 | ACGTTGGATGGTAATGAATCTGGCATCTCC | ACGTTGGATGGTAATAGTGCAAATGCCTGTC |
| 2325568 | ACGTTGGATGTGAATCTGGCATCTCCCATG | ACGTTGGATGGTAATAGTGCAAATGCCTGTC |
| 1998572 | ACGTTGGATGTCCTTTGAAAAGTAGTACTC | ACGTTGGATGTGAAATCTAACCCTGCGGTC |
| 1998573 | ACGTTGGATGACAGAATAGCTTTTTGGAAG | ACGTTGGATGCGGTCCAGGTAAAAGGACAG |
| Unknown | ACGTTGGATGAAACACTCCATTCTCTGCCC | ACGTTGGATGTTCTAGCTGTGAACTGGCAC |
| 1324061 | ACGTTGGATGTCAGAACATTGTGCCGTTTC | ACGTTGGATGTGCTCTTGAACTCTATCCTG |
| 2209726 | ACGTTGGATGCTTGTACTGGAAAGTAGCCC | ACGTTGGATGGGAGGATCTTGAGTCCTTTC |
| 2225100 | ACGTTGGATGCCAAAGCTTTACGCTTCTCC | ACGTTGGATGGACTCTGGAGGAAGTCTTAC |

TABLE 9

| SNP | | Term |
|---|---|---|
| Reference | Extend Probe | Mix |
| 3812851 | CCCTATACTAGAAAAGTCACCA | ACG |
| 3812850 | CATTAAAGTTCCTGCTTGAC | ACT |
| 2325555 | AGCCTTTCTGAAGAAGACAAG | ACT |
| 1535802 | GAAATTCACAACCAACCCTTG | ACT |
| 1535801 | AGATAGGCCTTTATAAACCCTTT | CGT |
| 718569 | CCATGGCTAATAGAGAATGTG | ACG |
| 2325556 | TTAGGTTTCAAGGAGAATG | ACT |
| 2274086 | CCTCCAAATTCTCATCTTTTCTT | ACT |
| 1886235 | TGCAAGAAAGAGTGGCAAAT | CGT |
| 1886234 | GCAATTGTAGGAAGGTAAATAA | ACT |
| 2325558 | TTTTTAAGTATGGGAAGATGAT | ACG |
| 2325559 | AACTTTGTGGAATGGAGTAA | ACT |
| 2325560 | TAAACAAATACAGATATTGAGCC | ACG |
| 3764134 | AAACTTTTGATTATTCATTCGATT | ACT |
| 3764133 | AACATTATGACAGACCTGTT | ACT |
| 2025426 | GTTTTCAATGTTTTCATAAACCTA | ACG |
| 2025425 | CTGCAGGCTCTGTAGAGCA | ACT |
| 1324059 | CTGAGTAAAGAAGCTGTTCAAG | ACT |
| 2325561 | AACCATTTACCACAACACAC | ACT |
| 2875666 | GTGCCAGTTCCAATCTT | ACT |
| 1408248 | GGCACACATTACATACTGTT | ACT |
| 1324058 | GGGTGCTGGCATTGACAGGCTA | CGT |
| 1011059 | GACAGGCTAAATGCTAAGTGAC | CGT |
| 1011058 | AACACTCTGGTTACTTCTTATTT | ACG |
| 2325563 | TGTATGGAATCGTGTTCTTTT | ACT |
| 2325564 | CAAAATCTGTGAAACATAGTTAT | ACT |
| 879800 | GTGAAATTGTTCATTTACTCTTTG | ACG |
| 2325565 | CCACTAGAATATTAAGCACCA | ACG |
| 255595 | GGCACATTAAAAGATGCTTC | ACT |
| 2875667 | TGTTTACACATGCACATTC | ACT |
| 2325566 | CCTGGCATTCTAGGGCT | ACT |
| 2325567 | ATCTGGCATCTCCCATGATA | ACG |
| 2325568 | CATCTCCCATGATACACA | ACG |
| 1998572 | CTTTGAAAAGTAGTACTCATATGT | ACT |
| 1998573 | GTTCATCCTTTGAAAAGTAG | ACT |
| Unknown | CATTCTCTGCCCTAATCTC | ACG |
| 1324061 | TTCCTGCTTTGGCAAATTCACG | ACT |
| 2209726 | AAGTAGCCCATGAGGTC | ACT |
| 2225100 | CCTCAAATGCCACCTAC | ACT |

Table 10 shows allelotyping results in female breast cancer and female control pools. Allele frequency is noted in the fifth and sixth columns for breast cancer pools and control pools.

TABLE 10

| | | Position in | | | Cancer | | | |
|---|---|---|---|---|---|---|---|---|
| Rs | Chromosome | SEQ ID | Gene | | Associated | Case | Control | |
| number | Position | NO: 1 | Location | Alleles | Allele | AF | AF | p-Value |
| 3812851 | 68305543 | 50479 | intron | G/A | G | 0.769 | 0.804 | 0.151 |
| 3812850 | 68305631 | 50567 | intron | A/G | A | 0.002 | 0.013 | 0.025 |
| 2325555 | 68306929 | 51865 | intron | A/G | G | 0.239 | 0.190 | 0.047 |
| 1535802 | 68313413 | 58349 | intragenic | C/G | G | 0.329 | 0.307 | 0.443 |
| | | | intron | | | 0.329 | 0.307 | 0.443 |
| | | | intragenic | | | 0.346 | 0.330 | 0.557 |
| 1535801 | 68313528 | 58464 | intragenic | A/T | A | 0.468 | 0.510 | 0.172 |
| | | | intron | | | 0.468 | 0.510 | 0.172 |
| 718569 | 68323615 | 68551 | intron | C/T | C | 0.638 | 0.676 | 0.188 |
| 2325556 | 68325483 | 70419 | intron | C/G | G | 0.823 | 0.810 | 0.590 |
| 2274086 | 68328078 | 73014 | intron | T/G | T | 0.529 | 0.562 | 0.270 |
| 1886235 | 68330306 | 75242 | intron | G/T | T | 0.832 | 0.828 | 0.860 |
| 1886234 | 68330569 | 75505 | intragenic | A/G | A | 0.473 | 0.506 | 0.273 |
| | | | intron | | | 0.473 | 0.506 | 0.273 |
| 2325558 | 68333131 | 78067 | intron | G/A | G | 0.664 | 0.670 | 0.825 |
| 2325559 | 68333329 | 78265 | intron | T/G | G | 0.070 | 0.033 | 0.006 |
| 2325560 | 68333354 | 78290 | intron | C/T | C | 0.694 | 0.716 | 0.430 |
| 3764134 | 68334020 | 78956 | coding- | A/C | A | 0.350 | 0.370 | 0.496 |
| | | | synon | | | | | |
| | | | reference | | | 0.350 | 0.370 | 0.496 |
| 3764133 | 68334260 | 79196 | intron | A/G | G | 0.772 | 0.718 | 0.038 |
| 2025426 | 68336950 | 81886 | intragenic | G/A | A | 0.575 | 0.527 | 0.110 |
| | | | intron | | | 0.575 | 0.527 | 0.110 |
| 2025425 | 68337463 | 82399 | intragenic | T/G | G | 0.746 | 0.737 | 0.745 |
| | | | intron | | | 0.746 | 0.737 | 0.745 |
| | | | intragenic | | | 0.744 | 0.746 | 0.950 |
| 1324059 | 68338444 | 83380 | intragenic | A/G | A | 0.712 | 0.720 | 0.776 |
| | | | intron | | | 0.712 | 0.720 | 0.776 |
| 2325561 | 68341709 | 86645 | intron | T/C | C | 0.310 | 0.298 | 0.668 |
| 2875666 | 68341768 | 86704 | intron | T/C | T | 0.175 | 0.199 | 0.314 |
| 1408248 | 68343198 | 88134 | intron | C/G | C | 0.290 | 0.338 | 0.085 |
| 1324058 | 68351480 | 96416 | intragenic | C/A | A | 0.716 | 0.663 | 0.057 |
| | | | intron | | | 0.716 | 0.663 | 0.057 |
| 1011059 | 68351493 | 96429 | intragenic | A/T | T | 0.148 | 0.107 | 0.038 |
| | | | intron | | | 0.148 | 0.107 | 0.038 |
| 1011058 | 68351599 | 96535 | intragenic | C/T | C | 0.759 | 0.836 | 0.002 |
| | | | intron | | | 0.759 | 0.836 | 0.002 |
| | | | intragenic | | | 0.754 | 0.833 | 0.002 |
| 2325563 | 68365984 | 110920 | intron | A/C | C | 0.353 | 0.340 | 0.645 |
| 2325564 | 68369181 | 114117 | intron | T/C | T | 0.645 | 0.660 | 0.595 |
| 879800 | 68372361 | 117297 | intron | C/T | T | 0.105 | 0.064 | 0.014 |
| 2325565 | 68372681 | 117617 | intron | C/T | C | 0.411 | 0.497 | 0.005 |
| 255595 | 68373934 | 118870 | intragenic | A/G | G | 0.694 | 0.630 | 0.024 |
| 2875667 | 68378804 | 123740 | intron | A/G | A | 0.177 | 0.196 | 0.412 |
| 2325566 | 68378856 | 123792 | intron | T/G | T | 0.208 | 0.233 | 0.326 |
| 2325567 | 68378940 | 123876 | intron | C/T | C | 0.264 | 0.306 | 0.121 |
| 2325568 | 68378944 | 123880 | intron | C/T | C | 0.974 | 0.992 | 0.024 |
| 1998572 | 68381749 | 126685 | intron | A/C | A | 0.268 | 0.279 | 0.676 |
| | | | mrna-utr | | | 0.268 | 0.279 | 0.676 |
| | | | unknown | | | 0.268 | 0.279 | 0.676 |
| 1998573 | 68381760 | 126696 | intron | A/C | A | 0.040 | 0.048 | 0.516 |
| | | | mrna-utr | | | 0.040 | 0.048 | 0.516 |
| | | | unknown | | | 0.040 | 0.048 | 0.516 |
| unknown | 68382054 | 126990 | intragenic | C/T | T | 0.715 | 0.714 | 0.961 |
| | | | intragenic | C/T | C | 0.703 | 0.722 | 0.506 |
| 1324061 | 68383003 | 127939 | intragenic | T/C | T | 0.401 | 0.402 | 0.964 |
| | | | intron | | | 0.401 | 0.402 | 0.964 |
| 2209726 | 68401210 | 146146 | intron | T/C | T | 0.200 | 0.225 | 0.302 |
| 2225100 | 68401376 | 146312 | intron | T/G | T | 0.170 | 0.211 | 0.079 |

The chromosome position is based on “build 31” of NCBI's GenBank. Some of the SNPs were allelotyped multiple times with different assays, e.g., using different PCR primers. Some of the SNPs have multiple annotations, therefore, there may be conflicting “sequence locations.” Those SNPs with allelotyping p-values of less than 0.04 are in bold text.

The full-length KLF12 gene consists of eight exons, while the truncated form consists of five exons. See Table 11:

TABLE 11
Full-Length and Truncated KLF12 Exons

| | | | | | | | SEQ ID | | |
|---|---|---|---|---|---|---|---|---|---|
| | | | | | | | NO: 1 | | |
| | SEQ ID NO: 1 | | NT_024524 | | | | Exon | NM_007249 | NM_016285 |
| Exon | Ensembl Build 31 | | NM_007249 (FL) | | NM_016285 (TR) | | length | Exon length | Exon length |
| 1 | 447728 | 447894 | 1 | 167 | 1 | 167 | 166 | 166 | 166 |
| 2 | 308979 | 309043 | 138853 | 138916 | 138853 | 138916 | 64 | 63 | 63 |
| 3 | 257970 | 258059 | 189836 | 189925 | 189836 | 189925 | 89 | 89 | 89 |
| 4 | 159816 | 160362 | 287533 | 288079 | 287533 | 288079 | 546 | 546 | 546 |
| 5 | 127141 | 127276 | 320619 | 320754 | 320619 | 321347 | 135 | 135 | 728 |
| 6 | 78927 | 78989 | 368906 | 368968 | 0 | 0 | 62 | 62 | 0 |
| 7 | 29357 | 29514 | 418381 | 418538 | 0 | 0 | 157 | 157 | 0 |
| 8 | 1 | 9660 | 438235 | 447894 | 0 | 0 | 9659 | 9659 | 0 |

In addition, the exons of KLF12 were sequenced using ABI's Dye-Terminator Cycle Sequencing. The sequencing of the amplified DNA was carried out on ABI 377 sequencers. The sequences of the amplification products were determined using automated dideoxy terminator sequencing reactions with a dye terminator cycle sequencing protocol. The sequence data were further evaluated to detect the presence of polymorphic variants within the amplified fragments. The polymorphism search was based on the presence of superimposed peaks in the electrophoresis pattern resulting from different bases occurring at the same position. The localization of the biallelic markers detected in the fragments of amplification are as shown below in Table 12:

TABLE 12

| Position in SEQ | | | Minor Allele |
|---|---|---|---|
| ID NO: 1 | Gene Position | Alleles | Frequency |
| 1 | Exon 7 | T/G | 0.10 |
| 1437 | Exon 7 | A/T | 0.05 |
| 1604 | Exon 7 | G/A | 0.10 |
| 2251 | Exon 7 | A/T | 0.05 |
| 2906 | Exon 7 | T/C | 0.20 |
| 2953 | Exon 7 | C/T | 0.20 |
| 3131 | Exon 7 | G/A | 0.05 |
| 3133 | Exon 7 | A/A | 0.00 |
| 3238 | Exon 7 | T/G | 0.20 |
| 3594 | Exon 7 | A/G | 0.10 |
| 4312 | Exon 7 | C/G | 0.11 |
| 4854 | Exon 7 | T/G | 0.05 |
| 5100 | Exon 7 | G/A | 0.11 |
| 5351 | Exon 7 | A/G | 0.11 |
| 5355 | Exon 7 | G/A | 0.11 |
| 78956 | Exon 5 | G/T | 0.50 |
| 96535 | Intron 4 | C/T | Incident SNP |
| 126990 | Intron 4 | T/C | 0.19 |
| 127370 | Intron 3 | A/G | 0.05 |
| 258062 | Exon 2 | G/A | 0.40 |
| 309011 | Exon 1; ATG | | |
| 309745 | Promoter | G/A | 0.14 |
| 310144 | Promoter | T/C | 0.17 |
| 310238 | Promoter | T/C | 0.11 |
| 310144 | Promoter | T/C | 0.17 |
| 310238 | Promoter | T/C | 0.11 |

The minor allele is set forth as the second allele in column four, “Alleles”, of Table 12.

Four of the SNPs set forth in Table 12 were genotyped as described in Example 2 using the primers and probes set forth in Tables 13 and 14. None of the four SNPs genotyped showed a significant association with breat cancer. See Table 15.

TABLE 13
PCR Primers

| Reference | Forward | Reverse |
|---|---|---|
| SNP ID | PCR primer | PCR primer |
| 259485 | ACGTTGGATGGCTGAATCTTTCTCTCTCCC | ACGTTGGATGTGTTTTGACTCTGACTGCCG |
| 128413 | ACGTTGGATGAAACACTCCATTCTCTGCCC | ACGTTGGATGTGTGAACTGGCACGAATGTC |
| 311168 | ACGTTGGATGAGGACCATTAGGATATCACC | ACGTTGGATGTCCAAATAGGGCAAATGGTC |
| 80379 | ACGTTGGATGAGACTGTATACTCACCAAGG | ACGTTGGATGTCTGTCTACAGGGTACATCC |

TABLE 14

| Reference | Extend | Term |
|---|---|---|
| SNP ID | Probe | Mix |
| 259485 | TCTCTCTCCCTTCTATTCT | ACG |
| 128413 | CTCCATTCTCTGCCCTAATCTC | ACG |
| 311168 | ATTGGGCTGGGGAGTTCT | ACT |
| 80379 | ACTTTTGATTATTCATTCGATT | ACT |

TABLE 15

| Reference | AF | AF | |
|---|---|---|---|
| SNP ID | F case | F control | p-value |
| 259485 | G = 0.646 | G = 0.622 | 0.402 |
| | A = 0.354 | A = 0.378 | |
| 128413 | T = 0.761 | T = 0.778 | 0.522 |
| | C = 0.239 | C = 0.222 | |
| 311168 | Not Polymorphic | | |
| 80379 | T = 0.554 | T = 0.523 | 0.313 |
| | G = 0.446 | G = 0.477 | |

### Example 4

### Screening Assay to Detect Inhibitors of KLF12

The following is an exemplary assay for finding inhibitors of KLF12. There are many assays known in the art for detecting inhibitors. See, e.g., Roth C et al. Genomics 2000 Feb. 1;63(3):384-90. Cells are transfected, transiently or often stably with the reporter construct described in Roth et al. Genomics 2000 Feb. 1;63(3):384-90. The cells often are chosen for minimal expression of endogenous KLF12, so that it can be externally introduced. Cells expressing the construct are co-transfected with a KLF12 expression vector or with vector lacking the KLF12 sequence (control). Both sets of cells are treated with the test compound and reporter gene activity is measured. Active compounds are selected based on their ability to prevent inhibition of the reporter gene expression when co-expressed with KLF12 and to have minimal effect on the control cells.

### Example 5

### KLF12 Expression Profile

Breast cells were profiled for AP-2a expression to determine if an inverse correlation exists between KLF12 and AP-2a levels. This relationship was seen for cells that do not express significant levels of the KLF12 truncated isoform (e.g., MCF-7, T-47D, Hbl-100). See FIG. 4. This result is consistent with the reported role of KLF12 as a transcriptional repressor of AP-2a. However, for breast cell lines MDA-MB-231 and MDA-MB-436, high KLF12 levels do not correlate with low AP-2a levels.

FIG. 4 shows the cumulative mRNA expression profile of the five breast cell lines (MCF-7, T47D, Hbl-100, MDA-MB-231 and MDA-MB436) from the 56-panel compared to normal breast tissue. The 56-panel consists of a subset of 56 cells that represent a plurality of cells from different human tissue types. KLF12 TR and AP-2a RT-PCR were done in a semi-automated mode in a volume of 10 μl while the KLF12 (both) RT-PCR was done manually in a total volume of 25 μl using Hotstar Taq™ from Qiagen, Inc. Any well-known expression profiling technique may be used.

### Example 6

### Inhibition of KLF12 Gene Expression by Transfection of Specific siRNAs

RNAi-based gene inhibition was selected as a rapid way to inhibit expression of KLF12 in cultured cells. SiRNA reagents were selectively designed to target truncated isoform of KLF12 (TR), or both isoforms. Algorithms useful for designing siRNA molecules specific for the KLF12 targets are disclosed at the http address www.dhramacon.com. siRNA molecules up to 21 nucleotides in length were utilized. Table 16 summarizes the features of two duplexes that were ordered from Dharmacon Research, Inc., and subsequently used in the assays described herein. A non-homologous siRNA reagent was used as a negative control.

TABLE 16
Duplex 21-mer siRNAs used for cell transfection

| | | | |
|---|---|---|---|
| | | | SEQ ID |
| siRNA | siRNA Target | Sequence Specificity | NO: |

| | | |
|---|---|---|
| siKLF1 | Targets both | AAGGGUCUCCAAACGUCCACA |
| | forms; exon2/3 | |
| | boundary | |
| siKLF4 | Targets trun- | AAGUAUCACAUUCACAGGAUG |
| | cated form | |
| | only; 3′ UTR | |
| siKLF1 | Non-homologous | AAUGCCACAGUACACCAGUCG |
| scrm | scrambled con- | |
| | trol | |

The siRNAs were transfected in cell lines MCF-7 and T-47D using Lipofectamine™ 2000 reagent from Invitrogen, Corp. 2.5 μg or 5.0 μg of siRNA was mixed with 6.25 μl or 12.5 μl lipofectamine, respectively, and the mixture was added to cells grown in 6-well plates. Their inhibitory effects on KLF12 gene expression were confirmed by precision expression analysis by MassARRAY (quantitativeRT-PCR hME), which was performed on RNA prepared from the transfected cells. See Chunming D. and Cantor C. PNAS 100(6):3059-3064 (2003). RNA was extracted from cells two days after transfection. RNA was extracted with Trizole reagent as recommended by the manufacturer (Invitrogen, Corp.) followed by cDNA synthesis using SuperScript™ reverse transcriptase. The level of KLF12 transcript during this exact experiment is shown in FIG. 5. Both siKLF1 and siKLF4 suppressed the levels of KLF12 transcript as compared to an siLamin control. Low levels of lipid control are non-characteristic. Results of the qRT-PCR-hME assay for KLF12 demonstrated higher that usual variability between experiments due to very low expression levels of KLF12.

The specificity of the RNAi effect was confirmed by transfecting siRNA with a sequence corresponding to a randomly scrambled sequence for siKLF1 (siKLF1 scrm). FIG. 11 shows that, like the other controls (siGL2 and lipid), siKLF1 scrm did not inhibit proliferation of MCF-7 cells, while siKLF1 completely inhibited proliferation as described in Example 7 below.

### Example 7

### Cell Proliferation

The siRNAs from Example 6 were transfected in cell lines grown in 6-well plates. Cells were trypsinized on the following day and distributed into 96-well plates. Wst-1 reagent was added on the indicated days and the absorbance at 650 nm and 450 nm was measured. The difference in absorbance between these 2 wavelengths is an indication of the metabolic activity in each well that was measured. Metabolic activity is directly proportional to the number of cells in each well.

Suppression of KLF12 mRNA levels correlated with decreased cell proliferation. The siRNA duplex siKLF1 suppressed proliferation of six non-related breast cancer cell lines (MCF-7, T-47D, Hbl-100, ZR-75-1, Hs 578, MDA-MB-435), while siRNA probe (siKLF4) was effective in some cell lines (MCF-7). See FIGS. 6, 7 and 8. This effect was confirmed by two different assays and by changes in cell morphology, as described herein.

A direct DNA measurement assay (see, e.g., CyQuant® from Molecular Probes, Inc.) was also employed in several experiments. In this experiment siKLF1 and siKLF4 both inhibited proliferation of T-47D cells 7 days after siRNA transfection. See FIG. 9. This confirms the results seen in the other experiment shown in FIG. 8 on day 7.

These results clearly implicate KFL12 gene expression in the regulation of proliferation of human breast cancer cells. Specifically, siKLF1 can potently suppress proliferation of at least five breast cancer lines (MCF-7, T-47D, ZR-75-1, Hs 578, MDA-MB-435), regardless of what isoform (FL or TR) is targeted. siKLF1 is also active against melanoma and prostate cancer lines, as described herein. In cases where only partial inhibition is achieved, a cocktail of siRNAs and/or repeated transfection may aid in completely stopping cell division.

Cell Morphology

Examination of cells by phase contrast microscopy suggests that cells that are affected by the siKLFs have started to assume an apoptotic morphology. See FIG. 10. The morphology of cells is consistent with the inhibitory effect of two siRNAs, siKLF1 and siKLF4, on proliferation. The cells are smaller and more rounded than the controls, siLucCy3 or siLaminA/C. This phenomenon was repeatedly observed. Staining of cells with annexin-FITC and propidium iodide confirmed that the cells are dying by apoptosis. See FIG. 12.

Other Cancer Cell Lines

In addition, it was shown that growth of at least two other non-breast tumor cell lines was suppressed via inhibition of KLF12 expression. siRNAs were tested against KLF12 in melanoma and prostate cancer cell lines using the cell proliferation assay described herein. siKLF1 was effective in decreasing proliferation of melanoma and prostate cancer lines: A2058 (SQC0143) and PC3 (SQC0065), respectively. See FIGS. 11A and 11B. It should be noted that repeated dosing or a cocktail of different siRNAs against KLF12 may be required for melanoma since the surviving cells resume at the same proliferative rate as the lipid control by day 6. This may be due to higher KLF12 level in these cells.

### Example 8

### In Vitro Production of KLF12 Polypeptides

KLF12 cDNA is cloned into a p1VEX 2.3-MCS vector (Roche Biochem) using a directional cloning method. A KLF12 cDNA insert is prepared using PCR with forward and reverse primers having 5′ restriction site tags (in frame) and 5-6 additional nucleotides in addition to 3′ gene-specific portions, the latter of which is often about twenty to about twenty-five base pairs in length. A Sal I restriction site is introduced by the forward primer and a Sma I restriction site is introduced by the reverse primer. The ends of KLF12 PCR products are cut with the corresponding restriction enzymes (i.e., Sal I and Sma I) and the products are gel-purified. The p1VEX 2.3-MCS vector is linearized using the same restriction enzymes, and the fragment with the correct sized fragment is isolated by gel-purification. Purified KLF12 PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the KLF12 template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The p1VEX 2.3 MCS vector includes a nucleotide sequence that encodes six consecutive histidine amino acids on the C-terminal end of the KLF12 polypeptide for the purpose of protein purification. KLF12 polypeptide is purified by contacting the contents of reaction device with resin modified with Ni²⁺ ions. KLF12 polypeptide is eluted from the resin with a solution containing free Ni²⁺ ions.

### Example 9

### Cellular Production of KLF12 Polypeptides

KLF12 nucleic acids are cloned into DNA plasmids having phage recombination cites and KLF12 polypeptides and polypeptide variants are expressed therefrom in a variety of host cells. Alpha-phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Corp.)).

A first step is to transfer the KLF12 nucleic acid insert into a shuttle vector that contains attL sites surrounding the negative selection gene, ccdB (e.g. pENTER vector, Invitrogen, Corp.). This transfer process is accomplished by digesting the KLF12 nucleic acid from a DNA vector used for sequencing, and to ligate it into the multicloning site of the shuttle vector, which will place it between the two attL sites while removing the negative selection gene ccdB. A second method is to amplify the KLF12 nucleic acid by the polymerase chain reaction (PCR) with primers containing attB sites. The amplified fragment then is integrated into the shuttle vector using Int and IHF. A third method is to utilize a topoisomerase-mediated process, in which the KLF12 nucleic acid is amplified via PCR using gene-specific primers with the 5′ upstream primer containing an additional CACC sequence (e.g., TOPO® expression kit (Invitrogen, Corp.)). In conjunction with Topoisomerase I, the PCR amplified fragment can be cloned into the shuttle vector via the attL sites in the correct orientation.

Once the KLF12 nucleic acid is transferred into the shuttle vector, it can be cloned into an expression vector having attR sites. Several vectors containing attR sites for expression of KLF12 polypeptide as a native polypeptide, N-fusion polypeptide, and C-fusion polypeptides are commercially available (e.g., pDEST (Invitrogen, Corp.)), and any vector can be converted into an expression vector for receiving a KLF12 nucleic acid from the shuttle vector by introducing an insert having an attR site flanked by an antibiotic resistant gene for selection using the standard methods described above. Transfer of the KLF12 nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the KLF12 polypeptide or polypeptide variants. These expression vectors are often transfected, for example, into murine-transfonned a adipocyte cell line 3T3-L1, (ATCC), human embryonic kidney cell line 293, and rat cardiomyocyte cell line H9C2.

Modifications can be made to the foregoing without departing from the basic aspects of the invention. Although the invention has been described in substantial detail with reference to one or more specific embodiments, those of skill in the art will recognize that changes can be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. A method for identifying a subject at risk of breast cancer, which comprises detecting the presence or absence of one or more polymorphic variations associated with breast cancer in a nucleic acid sample from a subject, wherein the one or more polymorphic variations are detected in a nucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c);
whereby the presence of the polymorphic variation is indicative of the subject being at risk of breast cancer.

2. The method of claim 1, which further comprises obtaining the nucleic acid sample from the subject.

3. The method of claim 1, wherein the one or more polymorphic variations are detected at one or more positions in SEQ ID NO: 1 is selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

4. The method of claim 1, wherein the one or more polymorphic variations are detected within a region spanning positions 78265 to 123880, spanning positions 78265 to 96535, or spanning positions 117297 to 123880 in SEQ ID NO: 1.

5. The method of claim 3, wherein a polymorphic variation is detected at position 78265 in SEQ ID NO: 1.

6. The method of claim 3, wherein a polymorphic variation is detected at position 96535 in SEQ ID NO: 1.

7. The method of claim 3, wherein a polymorphic variation is detected at position 117617 in SEQ ID NO: 1.

8. The method of claim 1, wherein the one or more polymorphic variations are detected at one or more positions in linkage disequilibrium with one or more positions in SEQ ID NO: 1 selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

9. The method of claim 1, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

10. The method of claim 1, wherein the subject is a human.

11. A method for identifying a polymorphic variation associated with breast cancer proximal to an incident polymorphic variation associated with breast cancer, which comprises:
identifying a polymorphic variation proximal to the incident polymorphic variation associated with breast cancer, wherein the polymorphic variation is detected in a nucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c) comprising the polymorphic variation;
determining the presence or absence of an association of the proximal polymorphic variant with breast cancer.

12. The method of claim 11, wherein the incident polymorphic variation is at a position in SEQ ID NO: 1 selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

13. The method of claim 1, wherein the incident polymorphic variation is within a region spanning positions 78265 to 123880, spanning positions 78265 to 96535, or spanning positions 117297 to 123880 in SEQ ID NO: 1.

14. The method of claim 11, wherein the proximal polymorphic variation is within a region between about 5 kb 5′ of the incident polymorphic variation and about 5 kb 3′ of the incident polymorphic variation.

15. The method of claim 11, which further comprises determining whether the proximal polymorphic variation is in linkage disequilibrium with the incident polymorphic variation.

16. The method of claim 11, which further comprises identifying a second polymorphic variation proximal to the identified proximal polymorphic variation associated with breast cancer and determining if the second proximal polymorphic variation is associated with breast cancer.

17. The method of claim 16, wherein the second proximal polymorphic variant is within a region between about 5 kb 5′ of the incident polymorphic variation and about 5 kb 3′ of the proximal polymorphic variation associated with breast cancer.

18. An isolated nucleic acid comprising a nucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c); and
(e) a nucleotide sequence complementary to the nucleotide sequences of (a), (b), (c), or (d);
wherein the nucleotide sequence comprises a nucleotide at a position in FIGS. 1A-1YYYY associated with breast cancer selected from the group consisting of a guanine at position 78265, a guanine at position 79169, a thymine at position 96429, a cytosine at position 96535, a thymine at position 117297, a cytosine at position 117617, a guanine at position 118870, and a cytosine at position 123880.

19. An oligonucleotide comprising a nucleotide sequence complementary to a portion of the nucleotide sequence of (a), (b), (c), or (d) in claim 18, wherein the 3′ end of the oligonucleotide is adjacent to a polymorphic variation associated with breast cancer.

20. A microarray comprising an isolated nucleic acid of claim 18 linked to a solid support.

21. An isolated polypeptide encoded by the isolated nucleic acid sequence of claim 18.

22. A method for identifying a candidate molecule that modulates cell proliferation, which comprises:
(a) introducing a test molecule to a system which comprises a nucleic acid comprising a nucleotide sequence selected from the group consisting of:
(i) a nucleotide sequence in FIGS. 1A-1YYYY or FIGS. 2A-2B;
(ii) a nucleotide sequence which encodes a polypeptide encoded by a nucleotide sequence in FIGS. 1A-1YYYY or FIGS. 2A-2B;
(iii) a nucleotide sequence which encodes a polypeptide that is 90% or more identical to the amino acid sequence encoded by a nucleotide sequence in FIGS. 1A-1YYYY or FIGS. 2A-2B;
(iv) a fragment of a nucleotide sequence of (i), (ii), or (iii); or introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate molecule that modulates cell proliferation.

23. The method of claim 22, wherein the system is an animal.

24. The method of claim 22, wherein the system is a cell.

25. The method of claim 22, wherein the nucleotide sequence comprises one or more polymorphic variations associated with breast cancer.

26. The method of claim 25, wherein the one or more polymorphic variations associated with breast cancer are at one or more positions in SEQ ID NO: 1 is selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

27. The method of claim 25, wherein the one or more polymorphic variations associated with breast cancer are in a region spanning positions 78265 to 123880, spanning positions 78265 to 96535, or spanning positions 117297 to 123880 in SEQ ID NO: 1.

28. A method for treating breast cancer in a subject, which comprises administering a candidate molecule identified by the method of claim 23 to a subject in need thereof, whereby the candidate molecule treats breast cancer in the subject.

29. A method for identifying a candidate therapeutic for treating breast cancer, which comprises:
(a) introducing a test molecule to a system which comprises a nucleic acid comprising a nucleotide sequence selected from the group consisting of:
(i) a nucleotide sequence in FIGS. 1A-1YYYY or FIGS. 2A-2B;
(ii) a nucleotide sequence which encodes a polypeptide encoded by a nucleotide sequence in FIGS. 1A-1YYYY or FIGS. 2A-2B;
(iii) a nucleotide sequence which encodes a polypeptide that is 90% or more identical to the amino acid sequence encoded by a nucleotide sequence in FIGS. 1A-1YYYY or FIGS. 2A-2B;
(iv) a fragment of a nucleotide sequence of (i), (ii), or (iii); or introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate therapeutic for treating breast cancer.

30. A method for treating breast cancer in a subject, which comprises contacting one or more cells of a subject in need thereof with a nucleic acid, wherein the nucleic acid comprises a nucleotide sequence selected from the group consisting of:
(a) a nucleotide sequence FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c); and
(e) a nucleotide sequence complementary to the nucleotide sequences of (a), (b), (c), or (d);
whereby contacting the one or more cells of the subject with the nucleic acid treats breast cancer in the subject.

31. The method of claim 30, wherein the nucleic acid is RNA or PNA.

32. The method of claim 31, wherein the nucleic acid is duplex RNA.

33. The method of claim 32, wherein a strand of the duplex RNA comprises a nucleotide sequence selected from the group consisting of

| | |
|---|---|
| AAGGGUCUCCAAACGUCCACA; | (SEQ ID NO: 138) |
| AAGUAUCACAUUCACAGGAUG | (SEQ ID NO: 139) |
| and | |
| AAUGCCACAGUACACCAGUCG. | (SEQ ID NO: 140) |

34. A method for treating breast cancer in a subject, which comprises contacting one or more cells of a subject in need thereof with a protein, wherein the protein is encoded by a nucleotide sequence which comprises a polynucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c);
whereby contacting the one or more cells of the subject with the protein treats breast cancer in the subject.

35. A method for treating breast cancer in a subject, which comprises:
detecting the presence or absence of one or more polymorphic variations associated with breast cancer in a nucleic acid sample from a subject, wherein the one or more polymorphic variation are detected in a nucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c) comprising the polymorphic variation; and
administering a breast cancer treatment to a subject in need thereof based upon the presence or absence of the one or more polymorphic variations in the nucleic acid sample.

36. The method of claim 35, wherein the one or more polymorphic variations are at one or more positions in SEQ ID NO: 1 selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

37. The method of claim 36, wherein the one or more polymorphic variations are in a region spanning positions 78265 to 123880, spanning positions 78265 to 96535, or spanning positions 117297 to 123880 in SEQ ID NO: 1.

38. The method of claim 35, which further comprises extracting and analyzing a tissue biopsy sample from the subject.

39. The method of claim 35, wherein the treatment is chemotherapy, surgery, radiation therapy, and combinations of the foregoing.

40. The method of claim 39, wherein the chemotherapy is selected from the group consisting of cyclophosphamide (Cytoxan), methotrexate (Amethopterin, Mexate, Folex), fluorouracil (Fluorouracil, 5-Fu, Adrucil), cyclophosphamide, doxorubicin (Adriamycin), and combinations of the foregoing.

41. The method of claim 40, wherein the combinations are selected from the group consisting of cyclophosphamide (Cytoxan), methotrexate (Amethopterin, Mexate, Folex), and fluorouracil (Fluorouracil, 5-Fu, Adrucil); cyclophosphamide, doxorubicin (Adriamycin), and fluorouracil; and doxorubicin and cyclophosphamide.

42. A method for detecting or preventing breast cancer in a subject, which comprises:
detecting the presence or absence of one or more polymorphic variations associated with breast cancer in a nucleic acid sample from a subject, wherein the polymorphic variation is detected in a nucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c) comprising the polymorphic variation; and
administering a breast cancer preventative or detection procedure to a subject in need thereof based upon the presence or absence of the one or more polymorphic variations in the nucleic acid sample.

43. The method of claim 42, wherein the one or more polymorphic variations are at one or more positions in SEQ ID NO: 1 is selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

44. The method of claim 43, wherein the one or more polymorphic variations are in a region spanning positions 78265 to 123880, spanning positions 78265 to 96535, or spanning positions 117297 to 123880 in SEQ ID NO: 1.

45. The method of claim 42, wherein the breast cancer detection procedure is selected from the group consisting of a mammography, an early mammography program, a frequent mammography program, a biopsy procedure, a breast biopsy and biopsy from another tissue, a breast ultrasound and optionally ultrasound analysis of another tissue, breast magnetic resonance imaging (MRI) and optionally MRI analysis of another tissue, electrical impedance (T-scan) analysis of breast and optionally of another tissue, ductal lavage, nuclear medicine analysis (e.g., scintimammography), BRCA1 and/or BRCA2 sequence analysis results, thermal imaging of the breast and optionally of another tissue, and a combination of the foregoing.

46. The method of claim 42, wherein the breast cancer preventative procedure is selected from the group consisting of one or more selective hormone receptor modulators, one or more compositions that prevent production of hormones, one or more hormonal treatments, one or more biologic response modifiers, surgery, and drugs that delay or halt metastasis.

47. The method of claim 46, wherein the selective hormone receptor modulator is selected from the group consisting of tamoxifen, reloxifene, and toremifene; the composition that prevents production of hormones is an aramotase inhibitor selected from the group consisting of exemestane, letrozole, anastrozol, groserelin, and megestrol; the hormonal treatment is selected from the group consisting of goserelin acetate and fulvestrant; the biologic response modifier is an antibody that specifically binds herceptin/HER2; the surgery is selected from the group consisting of lumpectomy and mastectomy; and the drug that delays or halts metastasis is pamidronate disodium.

48. A method of targeting information for preventing or treating breast cancer to a subject in need thereof, which comprises:
detecting the presence or absence of one or more polymorphic variations associated with breast cancer in a nucleic acid sample from a subject, wherein the polymorphic variation is detected in a nucleotide sequence selected from the group consisting of:
(a) a nucleotide sequence in FIGS. 1A-1YYYY or FIGS. 2A-2B;
(b) a nucleotide sequence which encodes a polypeptide encoded by a nucleotide sequence in FIGS. 1A-1YYYY or FIGS. 2A-2B;
(c) a nucleotide sequence which encodes a polypeptide that is 90% or more identical to the amino acid sequence encoded by a nucleotide sequence in FIGS. 1A-1YYYY or FIGS. 2A-2B;
(d) a fragment of a nucleotide sequence of (a), (b), or (c) comprising the polymorphic variation; and
directing information for preventing or treating breast cancer to a subject in need thereof based upon the presence or absence of the one or more polymorphic variations in the nucleic acid sample.

49. The method of claim 48, wherein the one or more polymorphic variations are at one or more positions in SEQ ID NO: 1 is selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

50. The method of claim 48, wherein the one or more polymorphic variations are in a region spanning positions 78265 to 123880, spanning positions 78265 to 96535, or spanning positions 117297 to 123880 in SEQ ID NO: 1.

51. The method of claim 48, wherein the information comprises a description of a breast cancer detection procedure, a chemotherapeutic treatment, a surgical treatment, a radiation treatment, a preventative treatment of breast cancer, and combinations of the foregoing.

52. A composition comprising a breast cancer cell and an antibody that specifically binds to a protein, polypeptide or peptide encoded by a nucleotide sequence identical to or 90% or more identical to a nucleotide sequence in FIGS. 1A-1YYYY or FIGS. 2A-2B.

53. A composition comprising a breast cancer cell and a RNA, DNA, PNA or ribozyme molecule comprising a nucleotide sequence identical to or 90% or more identical to a portion of a nucleotide sequence in FIGS. 1A-1YYYY or FIGS. 2A-2B.

54. The composition of claim 53, wherein the RNA molecule is a short inhibitory RNA molecule.

55. A method of selecting a subject that will respond to a treatment of breast cancer, which comprises:
detecting the presence or absence of one or more polymorphic variations associated with breast cancer in a nucleic acid sample from a subject, wherein the polymorphic variation is detected in a nucleotide sequence selected from the group consisting of:
(a) the nucleotide sequence of FIGS. 1A-1YYYY or FIGS. 2A-2B;
(b) a nucleotide sequence which encodes a polypeptide consisting of an amino acid sequence encoded by a nucleotide sequence in FIGS. 1A-1YYYY or FIGS. 2A-2B;
(c) a nucleotide sequence which encodes a polypeptide that is 90% or more identical to an amino acid sequence encoded by a nucleotide sequence in FIGS. 1A-1YYYY or FIGS. 2A-2B; and
(d) a fragment of a nucleotide sequence of (a), (b), or (c) comprising the polymorphic variation; and
selecting a subject that will respond to the breast cancer treatment based upon the presence or absence of the one or more polymorphic variations in the nucleic acid sample.

56. The method of claim 55, wherein the one or more polymorphic variations are at one or more positions in SEQ ID NO: 1 is selected from the group consisting of 50479, 50567, 51865, 58349, 58464, 68551, 70419, 73014, 75242, 75505, 78067, 78265, 78290, 78956, 79196, 81886, 82399, 83380, 86645, 86704, 88134, 96416, 96429, 96535, 110920, 14117, 117297, 117617, 118870, 123740, 123792, 123876, 123880, 126685, 126696, 126990, 127939, 146146, and 146312.

57. The method of claim 55, wherein the one or more polymorphic variations are in a region spanning positions 78265 to 123880, spanning positions 78265 to 96535, or spanning positions 117297 to 123880 in SEQ ID NO: 1.
